(19) United States
(12) Patent Application Publication
(10) Pub. No.: US 2022/0175787 A1
(43) Pub. Date: Jun. 9, 2022

(54) PATIENT SELECTION FOR ENHANCEMENT OF ANTI-TUMOR IMMUNITY IN CANCER PATIENTS

(71) Applicant: G1 Therapeutics Inc., Research Triangle Park, NC (US)

(72) Inventors: Patrick Joseph Roberts, Durham, NC (US); Anne Lai, Research Triangle Park, NC (US); Jessica Sorrentino, Durham, NC (US)

(73) Assignee: G1 Therapeutics Inc., Research Triangle Park, NC (US)

(21) Appl. No.: 17/554,940

(22) Filed: Dec. 17, 2021

Related U.S. Application Data
(63) Continuation of application No. PCT/US2020/038557, filed on Jun. 18, 2020.
(60) Provisional application No. 62/863,153, filed on Jun. 18, 2019.
(60) Provisional application No. 62/907,375, filed on Sep. 27, 2019.

(51) Int. Cl. A61K 31/522; A61K 45/06; A61P 35/00

(52) U.S. Cl. CPC A61K 31/522; A61P 35/00; A61K 45/06

(57) ABSTRACT

A method for increasing the progression free survival or overall survival of a patient with cancer comprising: determining if the cancer has a surrounding microenvironment that is favorable to immune modulation; determining if the chemotherapy regimen induces immunogenic cell death, and if both are yes, administering an effective amount of a CDK 4/6 inhibitor selected from Compounds I, II, III, IV, or V, or a pharmaceutically acceptable salt thereof, wherein the CDK4/6 inhibitor is administered prior to the administration of the chemotherapy or optionally prior to and concurrently with chemotherapy; and, wherein the increase in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field.

### CROSS REFERENCE TO RELATED APPLICATIONS

This application is a continuation of International Patent Application No. PCT/US2020/038557, filed on Jun. 18, 2020, which claims the benefit of U.S. Provisional Application 62/863,153, filed on Jun. 18, 2019; and U.S. Provisional Application 62/907,375, filed on Sep. 27, 2019; the entirety of each of these applications is hereby incorporated by reference for all purposes.

### FIELD OF THE INVENTION

This invention is in the field of cancer therapy, and provides methods of selecting patients for advantageous and directed cancer treatment that includes the administration of a cyclin dependent kinase (CDK) 4/6 inhibitor in conjunction with chemotherapy, based on patient and cancer profiles as further described herein. It has been discovered that when a specified subsection of cancer patients is administered a CDK 4/6 inhibitor in conjunction with chemotherapy, this selected patient population exhibits a progression free survival benefit and/or an overall survival benefit. This result can in some embodiments be achieved without the use of an immune checkpoint inhibitor, such as an anti-PD-1, anti-PD-L1, or anti-CTLA4 agent such as an antibody. It has also been discovered that when a different specified subsection of cancer patients is administered a CDK 4/6 inhibitor in conjunction with chemotherapy, a myelopreservation effect is achieved that spares immune cells and can result in a higher proportion of T and or B cells than without the therapy, perhaps without achieving an overall survival, but with an enhanced patient experience and quality of life.

### BACKGROUND

The tumor microenvironment (TME) consists of different cellular and non-cellular components in and around a tumor. The TME has been recognized to play a significant role in tumor progression. The TME shapes tumor evolution (whether the tumor regresses, develops resistance, evades the immune system and/or metastasizes) and consequently impacts patient outcomes. Chen et al., New horizons in tumor microenvironment biology: challenges and opportunities. BMC Med. Mar. 5, 2015; 13:45. doi: 10.1186/s12916-015-0278-7. An association has been observed between the levels of tumor infiltrating immune cells, key components of the TME, and patient prognosis: a colorectal cancer study showed that higher levels of tumor infiltrating CD3+ immune cells were associated with better disease-free survival. Galon et al., Type, density, and location of immune cells within human colorectal tumors predict clinical outcome. Science. Sep. 29, 2006; 313(5795):1960-4.

It has recently been appreciated that the action of chemotherapy is very complex, having an effect not just on the tumor, but also on the patient's immune cells that normally play a major role in protecting the body from diseased cells. Therefore, chemotherapy protocols should take into account not just the effect on the tumor, but also on the tumor microenvironment.

It has also been discovered that certain chemotherapies, but not all, are able to trigger a pathway in the tumor cell referred to as “immunogenic cell death” (“ICD”) (see generally, Locy, H., et al., Immunomodulation of the Tumor Microenvironment: Turn Foe Into Friend, Frontiers in Immunology, 2018; 9: 2090). ICD is a form of regulated cell death that induces the release of tumor associated antigens and triggers an anti-tumor immune response. Id. ICD involves the release of Damage-Associated Molecular Pathways (“DAMPS”) that alert the host's immune system that the cell is damaged. There are six DAMPs that facilitate cell death: calreticulin (“CRT”), high mobility group box 1 (HMGB1), extracellular ATP, type I interferon, cancer cell-derived nucleic acids and ANXA1. These DAMPs determine the strength and durability of the ICD anti-tumor response. See also, Wang, et al, Immunogenic effects of chemotherapy induced tumour cell death, Genes & Diseases (2018) 5, 194-203.

Chemotherapeutic agents may also induce an immunogenic effect by disrupting strategies that tumors use to evade the immune response. See, e.g., Emens et al., The Interplay of Immunotherapy and Chemotherapy: Harnessing Potential Synergies. Cancer Immunol Res; 3(5) May 2015. For example, chemotherapy can modulate distinct features of tumor immunobiology in a drug-, dose-, and schedule-dependent manner, and distinct chemotherapy drugs may modulate the intrinsic immunogenicity of tumor cells through a variety of mechanisms (see, e.g., Chen G, Emens L A. Chemoimmunotherapy: reengineering tumor immunity. Cancer Immunol Immunother 2013; 62:203-16). Chemotherapy can also enhance tumor antigen presentation by upregulating the expression of tumor antigens themselves, or of the MHC class I molecules to which the antigens bind. Alternatively, chemotherapy may upregulate costimulatory molecules (B7-1) or downregulate coinhibitory molecules (PD-L1/B7-H1 or B7-H4) expressed on the tumor cell surface, enhancing the strength of effector T-cell activity. Chemotherapy may also render tumor cells more sensitive to T cell-mediated lysis through fas-, perforin-, and Granzyme B-dependent mechanisms.

In addition, recent insight has been gained on the basic mechanisms of tumor-immune system interactions, allowing for the development of a tumor classification system based on the characteristics of an individual tumor's microenvironment in relation to immune effector cell populations and the presence or absence of certain immunogenic biomarkers and signals. In 2009, Camus et al, reported research on colorectal cancer using the categories hot, altered and cold. The 2-year relapse data for these tumors was 10%, 50% and 80%. Camus, M., et al., Coordination of intratumoral immune reaction and human colorectal cancer recurrence, Cancer Research 69, 2685-2693 (2009). They further classified the altered tumors as excluded or immunosuppressed. They found that in some tumors, T cells were found at the invasive margin but could not infiltrate (thus altered excluded), which allowed the tumor to protect itself. In other cases, tumors had a low degree of immune infiltration, which suggested a low degree of margin barriers but an immunosuppressed environment (thus altered immunosuppressed). This categorization of tumors is now becoming accepted in the field for not just colorectal cancer but also other cancers as a means to predict progression.

Galon and Bruni expanded on the four-category classification of tumors as hot, altered-excluded, altered-immunosuppressed and cold, to facilitate research and communications. Specifically, the category stratifications are based on type, density and location of immune cells within the tumor site (see FIG. 7a). The authors classify tumors according to immune infiltration instead of cancer type, with a scoring system (“Immunoscore”) based on the quantification of two lymphocyte populations (CD3 and CD8) both at the tumor center and the invasive margin. The score ranges from I0 (low densities, such as absence of both cell types in both regions) to 14 (high immune cell types in both locations). 14 tumors are considered “hot” and I0 tumors are “cold”. Tumor progression (T stage) and invasion (N stage) were reported to be dependent on this pre-existing adaptive intratumoral immunity. More frequently, researchers are now looking at the nature, density, immune functional orientation, and distribution of immune cells in the tumor. See Galon, J., and Bruni, D., Approaches to treat immune hot, altered and cold tumours with combination immunotherapies”, Nature Reviews Drug Discovery (18), March 2019, 197-218.

As reported by Galon, the basic characteristics of hot immune tumors are (i) a high degree of T cell and cytotoxic T cell infiltration and (ii) checkpoint activation or impaired T-cell functions. Altered-immunosuppressed immune tumors are categorized by (i) poor, but not absent, T-cell and cytotoxic T-cell infiltration, (ii) presence of soluble inhibitory mediators, (iii) the presence of immune suppressive cells and (iv) presence of T-cell checkpoints. The characteristics of altered-excluded immune tumors are (i) no meaningful T cell infiltration inside the tumor and an accumulation of T cells at tumor borders, (ii) activation of oncogenic pathways, (iii) epigenetic regulation and reprogramming of the tumor microenvironment and (iv) aberrant tumor vasculature and/or stroma and (v) hypoxia. The characteristics of a cold tumor are (i) absence of T cells within the tumor and at the tumor edges and (ii) failed T cell priming (i.e., poor, little or no antigen presentation, low tumor mutational burden and/or intrinsic insensitivity to T cell killing). Cold tumors can also show a low expression of PD-L1.

As shown in FIG. 6 herein, and reported on page 204 of their March 2019 Nature Reviews article (see FIG. 3 in Galon et al.), Galon et al. present a comprehensive wheel illustration of the four categories of tumors, the mechanisms used by the tumor cells to protect themselves, and the drugs/therapies that can be used to break through the protection.

Thorsson et al., identified six immune subtypes that encompass multiple tumor types based on extensive immunogenomic analysis of more than 10,000 tumors comprising 33 diverse cancer types. See Thorsson et al., “The Immune Landscape of Cancer,” Immunity 48, 812-830, 2018. The six immune subtypes are: C1—“Wound Healing” which is characterized by a high proliferation rate, high angiogenesis gene expression and a Th2 cell bias to the adaptive immune infiltrate; C2—“IFN-γ Dominant” which is characterized by the highest M1/M2 macrophage polarization, a strong CD8 signal and high TCR diversity; C3—“Inflammatory,” which is characterized by elevated Th17 and Th1 genes, low to moderate proliferation, low aneuploidy and overall somatic copy number alterations; C4—“Lymphocyte Depleted,” which is characterized by prominent macrophage signature with Th1 suppression and high M2 response; C5—“Immunologically Quiet,” which is characterized by a low lymphocyte response and a high macrophage response dominated by M2; and C6—“TGF-β Dominant,” which is characterized by a mixed tumor subgroup with high TGF-β and lymphocytic infiltration. Thorsson et al. noted that immune subtypes associated with overall survival (OS) and progression-free interval (PFI), with a cancer that fell within the C3 classification had the best prognosis, while cancers with a C2 or C1 classification had a less favorable outcome despite having a substantial immune component, while the more mixed-signature subtypes, C4 and C6, had the least favorable outcome.

Ayers et al., analyzed gene expression profiles (GEPs) using RNA from pretreatment baseline tumor samples of PD-1 treated patients and identified immune-related signatures correlating with clinical activity across 9 cancer types. See Ayers et al., “IFN-γ-related mRNA profile predicts clinical response to PD-1 blockade. J Clin Invest. 2017; 127(8):2930-2940. They found that the T cell-inflamed GEP contained IFN-γ-responsive genes related to antigen presentation, chemokine expression, cytotoxic activity, and adaptive immune resistance, and these features were necessary, but not always sufficient, for attaining a clinical benefit from the use of a checkpoint inhibitor. They identified a subset of six genes (“IFN-γ Signature”) and a further 18 genes (“Expanded Immune Signature”) whose expression profile provided a predictive value for determining the efficacy of PD-1-/PD-L1-directed monoclonal antibody treatment.

Despite progress in the area of understanding more about the effect of chemotherapy on the tumor microenvironment and categorizing tumors to increase understanding and patient outcomes, more research and discoveries are clearly needed to accurately select the patient population that will benefit from cancer therapy, and what kind of benefit can be achieved. The complexity and number of factors involved in advancing cancer therapy make this goal difficult and predictions challenging.

One goal is to be able to select the patient population for which the therapy may result in a progression free survival benefit and/or an overall survival benefit.

Another goal is to select a patient population for which therapy may result in a myelopreservation effect that protects immune cells, with or without a progression free survival or overall benefit, but with an enhanced patient experience or quality of life.

### SUMMARY OF THE INVENTION

This invention addresses the problem of patient selection to achieve certain cancer therapy outcomes when the patient is administered a cyclin dependent kinas 4/6 inhibitor in combination with chemotherapy.

It has been discovered that when a specified subsection of cancer patients is administered a CDK 4/6 inhibitor in conjunction with chemotherapy, this selected patient population exhibits a progression free survival benefit and/or an overall survival benefit. This result can in some embodiments be achieved without the use of an immune checkpoint inhibitor, such as an anti-PD-1, anti-PD-L1, or anti-CTLA4 agent such as an antibody. For example, where a cancer patient has a tumor that exhibits specific characteristics as described herein according to the Ayer's interferon-γ signature, the Ayer's expanded immune signature or the Thorsson et al Six Class Immune Signature, the patient population when administered a CDK 4/6 inhibitor in conjunction with chemotherapy is more likely to achieve a progression free survival or overall survival benefit. In one embodiment, the tumor has is interferon-γ (IFN-γ) dominant according to Thorsson's Six Class Immune Signature, or a high IFN-γ signature or expanded immune signature according to the Ayer's IFN-γ Signature Score or Expanded Immune Signature Score.

It has also been discovered that when a different specified subsection of cancer patients is administered a CDK 4/6 inhibitor in conjunction with chemotherapy, a myelopreservation effect is achieved in that selected patient population that spares immune cells and can result in a higher proportion of T and or B cells than without the therapy. In one embodiment, the different specified subsection of cancer patients wherein a myelopreservation effect is achieved is not small cell lung carcinoma. This patient population includes those with cancers that are not particularly immunogenic or sensitive to immune modulation, according to the characterizations as described in the Background or otherwise herein. In one embodiment, the cancer is poorly immunogenic and PD-L1 expression is relatively low (about less than 50%, 40% or even 30% that of normal expression). In another embodiment, the tumor has a reduced expression of major histocompatibility complex class I and class II molecules, a known immune escape mechanism, reflecting a less immunogenic environment.

This invention therefore provides a means to determine the outcome of therapy, and thus provides therapeutic protocols, using the appropriate selection of the combination of tumor type, chemotherapy type, and anti-cyclin dependent kinase (CDK) therapy and dosage regimen to maximize an anti-tumor immunity. The benefit can be a reversal of T-cell exhaustion, enhancement of immune cell activation including T cells, the formation of immunological memory, and/or reduction of immunosuppression in addition to enhancing general immunosurveillance. This result in some embodiments may be achieved without the use of an immune checkpoint inhibitor, such as an anti-PD-1, anti-PD-L1 or anti-CTLA4 agent such as an antibody. Importantly, the ability to extend progression free survival and/or overall survival without the need to administer a checkpoint inhibitor compound may reduce potential side-effects associated with immune checkpoint inhibitor treatments, including pneumonitis, hyperthyroidism, hypothyroidism, kidney infections, and immune-mediated rashes, including Stevens-Johnson syndrome (SJS), toxic epidermal necrolysis (TEN), exfoliative dermatitis, and bullous pemphigoid.

Specifically, it has been discovered through human clinical trials that progression free survival and/or overall survival can be improved when a cancer that is highly immunogenic, for example, a hot tumor (as defined in Galon, J., and Bruni, D., Approaches to treat immune hot, altered and cold tumours with combination immunotherapies”, supra, incorporated herein by reference and discussed further below), high IFN-γ expression, or other acceptable indicator of immunogenic susceptibility is treated with a chemotherapy that causes an immune-mediated response including, but not limited to, immunogenic cell death and/or regulatory T-cell (Treg cell) suppression, in combination with a short acting CDK4/6 inhibitor administered at least prior to the administration of the chemotherapy or alternatively, administered both prior to and concurrently with the chemotherapy. When a cancer therapy includes these three components in the appropriate dosage regimen, there is an immuno-oncology effect that promotes progression free survival and/or overall survival by alteration of the milieu of T-cells away from an immunosuppressive environment (i.e., Treg cells) and toward an enhancement of T-cell activity and an increase in cytotoxic T cells (CD8+ cells). In some embodiments, the CDK4/6 inhibitor is further administered in a maintenance-type therapeutic regimen, wherein the CDK4/6 inhibitor is administered as a single agent without chemotherapy at a regular dosing, for example but not limited to, once a week, once every two weeks, once every three weeks, once a month, or once every six weeks following the completion of chemotherapy treatment. In some embodiments, the CDK4/6 inhibitor is further administered with the chemotherapeutic agent in a maintenance-type therapeutic regimen, wherein the CDK4/6 inhibitor is administered with a lower dose of chemotherapy at a regular dosing, for example but not limited to, once a week, once every two weeks, once every three weeks, once a month, once every six weeks, once every two months, once every three months, once every four months, once every five months, or once every six months following the completion of the initial chemotherapy treatment regimen.

In an alternative embodiment, progression free survival and/or overall survival can be improved when a cancer that is categorized as altered-excluded or altered immunosuppressed according to the Galon et al. scoring system is treated with a chemotherapy that enhances an immune mediated anti-tumor response, including but not limited to a chemotherapy that induces immunogenic cell death, in combination with a short acting CDK4/6 inhibitor administered at least prior to the administration of the chemotherapy, or alternatively administered both prior to and concurrently with the chemotherapy. In some embodiments, the CDK4/6 inhibitor is further administered in a maintenance-type therapeutic regimen, wherein the CDK4/6 inhibitor is administered as a single agent without chemotherapy at a regular dosing, for example but not limited to, once a week, once every two weeks, once every three weeks, once a month, or once every six weeks following the completion of chemotherapy treatment. In some embodiments, the CDK4/6 inhibitor is further administered with the chemotherapeutic agent in a maintenance-type therapeutic regimen, wherein the CDK4/6 inhibitor is administered with a lower dose of chemotherapy at a regular dosing, for example but not limited to, once a week, once every two weeks, once every three weeks, once a month, once every six weeks, once every two months, once every three months, once every four months, once every five months, or once every six months following the completion of the initial chemotherapy treatment regimen.

In certain embodiments, the short acting CDK 4/6 inhibitor is selected from:

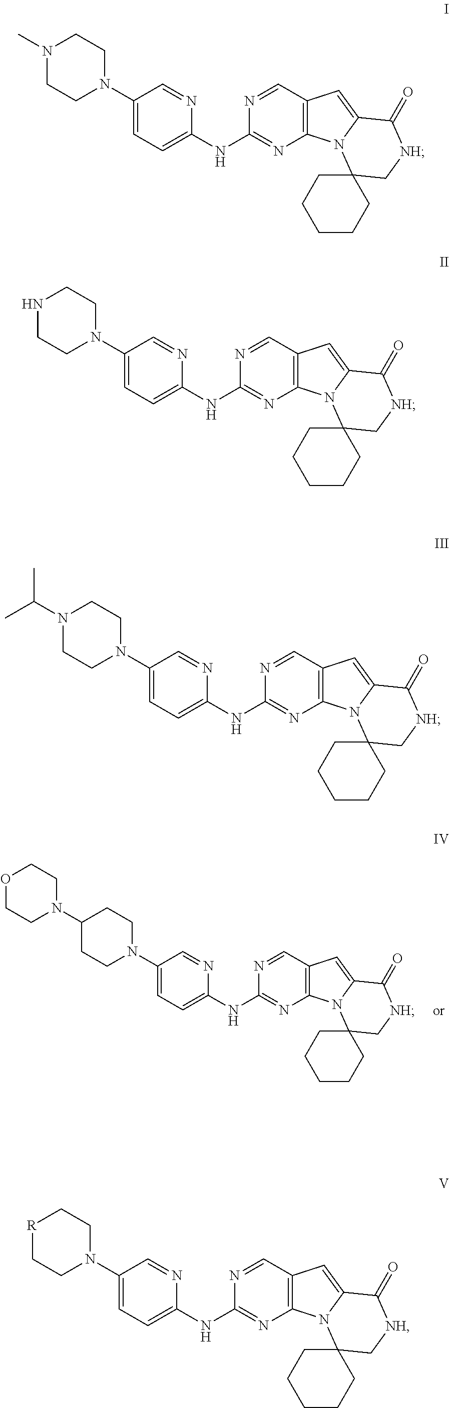

wherein R is C(H)X, NX, C(H)Y, or C(X)₂,

where X is straight, branched or cyclic C₁ to C5 alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and

Y is NR₁R₂ wherein R₁ and R₂ are independently X, or wherein R₁ and R₂ are alkyl groups that together form a bridge that includes one or two heteroatoms (N, O, or S);

and wherein two X groups can together form an alkyl bridge or a bridge that includes one or two heteroatoms (N, S, or O) to form a spiro compound, or a pharmaceutically acceptable salt thereof.

Cytotoxic chemotherapies generally do not differentiate between replicating healthy cells and cancer cells—killing both indiscriminately, including important stem cells in the bone marrow that produce white blood cells, red blood cells, and platelets. This chemotherapy-induced bone marrow damage is known as myelosuppression. When white blood cells, red blood cells and platelets become depleted, patients receiving chemotherapy are at an increased risk of infection, experience anemia and fatigue, and are at an increased risk of bleeding. Myelosuppression often requires the administration of rescue interventions such as growth factors and blood or platelet transfusions, and may also result in chemotherapy dose delays and reductions. It can also result in more hospital and doctor visits—burdening both the patient and the healthcare system, and increasing the risk to the patient. A myelopreservation agent is one that preserves hematopoietic stem cells, white blood cells, red blood cells and/or platelets in a situation (such as chemotherapy) in which such cells would be otherwise stressed, damaged or killed.

Compound I, also known as “trilaciclib” and developed by G1 Therapeutics, Inc., is currently being investigated in a number of human clinical trials for parenteral use as a myelopreservation agent administered via intravenous injection before chemotherapy with 1) gemcitabine and carboplatin in metastatic triple negative breast cancer (mTNBC), 2) topotecan in advanced staged small cell lung carcinoma (SCLC), 3) carboplatin and etoposide in SCLC, and 4) carboplatin, etoposide, and the PD-L1 immune checkpoint inhibitor atezolizumab (Tecentriq®) in SCLC.

Compound III, also known as “lerociclib” and developed by G1 Therapeutics, Inc., is currently being investigated in a number of human clinical trials as an antineoplastic agent, typically via continuous administration such as daily administration (with time off as necessary in the judgement of the healthcare provider) to treat 1) EGFR-mutant non-small cell lung carcinoma in combination with the EGFR inhibitor osimertinib (Tagrisso®), and 2) ER+, HER2− breast cancer in combination with fulvestrant.

As provided herein, the examples and discussion below is provided using trilaciclib or a pharmaceutically acceptable salt thereof, as the exemplary compound. In alternative embodiments, one of the other short acting CDK4/6 inhibitors described above may be used, including for example, lerociclib. In yet another embodiment, palbociclib, or another selective CDK 4/6 inhibitor such as abemaciclib or ribociclib is used. This is not a representation that any of these compounds are equivalent to trilaciclib in performance or effect, but instead, are considered alternative embodiments with potential alternative treatment effects, dosages or outcomes.

It was surprising to discover that the human clinical trials using trilaciclib as a myelopreservation agent to preserve hematopoietic progenitor and stem cell viability during chemotherapy, actually instead resulted in an improved overall survival across the entire patient population with statistical significance in triple negative breast cancer (TNBC). Therefore, it was quite unexpected that the human clinical trial had a different, and better, outcome than designed and anticipated. As shown in Examples 2, 3, and 4, the effects are even greater when the immunogenicity of the individual tumor is accounted for. This unexpected immuno-oncology effect is the basis for the present invention.

In contrast, trilaciclib when used as a myelopreservation agent to treat small cell lung cancer, generally considered immunologically cold cancer—and thus less favorable to an induced immunological response—in combination with etoposide and carboplatin performed as designed, with a statistically significant myelopreservation effect, but not a statistically significant improvement in progression free survival or overall survival across the patient population. Reviewing the clinical trial data, however, indicates that within a sub-population of responders, significant immunological activity, most notably the expansion of new T-cell clones, was observed in those patients receiving trilaciclib (see Example 5, FIGS. 11-14). Importantly, these same patients with observed increases in clonal expansion of T-cells also experienced increased overall survival.

The nonclinical and clinical data presented herein indicate that the antitumor efficacy benefit of trilaciclib is an immune-mediated phenomenon, where both the type of chemotherapy and tumor are relevant to outcome. Chemotherapy that induces an immune-mediated response, for example immunogenic cell death, and tumors with a microenvironment more favorable to immune modulation support trilaciclib's antitumor efficacy.

Furthermore, the clinical data indicates that factors such as IFN-γ signaling and the associated biology of T-cell cytolytic activity, antigen presentation, and chemokine production play a significant role in the anti-tumor efficacy of trilaciclib. Importantly, as described herein, the factors determining the potential effectiveness of CDK4/6 antitumor efficacy are measurable prior to the initiation of therapy, providing for an effective and reproducible determination of potential effectiveness and implementation of a therapeutic regimen capable of extending overall and/or progression free survival.

For example, although SCLC is characterized by a high degree of genomic instability and smoking-associated mutational profile, SCLC tumors have significantly reduced levels of both major histocompatibility complex class I and class II complexes, a known method of escaping antitumor immunity (which makes it an immunologically “cold-like” tumor) (Semenova et al., Origins, genetic landscape, and emerging therapies of small cell lung cancer. Genes Dev 2015; 29: 1447-62). Therefore, in SCLC, trilaciclib acts to reduce chemotherapy-induced myelosuppression, without necessarily improving on antitumor efficacy across the patient population. By contrast, TNBC is generally genomically unstable, and the tumor microenvironment may be more immunogenic or “hot-like” when treated with gemcitabine, a strong ICD-agent (see, e.g., Park et al., How shall we treat early triple-negative breast cancer (TNBC): from the current standard to upcoming immuno-molecular strategies. ESMO Open 2018; 3 (suppl 1): e000357), leading to improved antitumor efficacy and extended overall survival.

Specifically, as described in the Examples below, at an initial data cutoff date of May 15, 2019, clinically meaningful improvement in antitumor efficacy was established when adding trilaciclib to a gemcitabine/carboplatin (GC) schedule to treat mTNBC (both dosing schedules) compared with GC alone. In particular, initial data cut-off showed a significant increase in median overall survival from 12.6 months with GC alone (Group 1 G/C therapy (Days 1 and 8 of 21-day cycles) 20.1 months (Group 2: G/C therapy (Days 1 and 8) plus trilaciclib administered IV on Days 1 and 8 of 21-day cycles) and 17.8 months (Group 3: G/C therapy (Days 2 and 9) plus trilaciclib administered IV on Days 1, 2, 8, and 9 of 21-day cycles) with the addition of trilaciclib (see Table 5; FIG. 2). At a follow up data cut-off date of May 15, 2020, median overall survival (OS) (95% CI) was 12.6 (6.3, 15.6) months in group 1, median OS has not yet been reached (NR-not reached)(due to extended patient population survival) (10.2, NR) in group 2 (HR=0.31, P=0.0016), and 17.8 (12.9, 32.7) months in group 3 (HR=0.40, P=0.0004). For groups 2 and 3 combined, median OS was 19.8 (14.0, NR) months (HR=0.37, P<0.0001 versus group 1). Importantly, there were no differences in overall response rate (ORR), progression free survival (PFS), or OS between tumors categorized as CDK4/6-replication independent or indeterminate.

The median overall survival for gemcitabine/carboplatin (GC) alone is consistent with published literature for patients with mTNBC treated in a similar setting (see O'Shaughnessy et al., Phase III study of iniparib plus gemcitabine and carboplatin versus gemcitabine and carboplatin in patients with metastatic triple-negative breast cancer. J Clin Oncol 2014; 32: 3840-47). In a phase 3 study of iniparib plus GC versus GC alone in patients who had received 0-2 prior chemotherapy regimens for metastatic disease, median overall survival among 258 patients treated with GC alone was 11.1 months (id.) Similarly, in a recent study of combination chemotherapy for the first-line treatment of patients with mTNBC, median OS was 12.1 months with GC (Yardley et al., nab Paclitaxel plus carboplatin or gemcitabine versus gemcitabine plus carboplatin as first-line treatment of patients with triple-negative metastatic breast cancer: results from the tnAcity trial. Ann Oncol 2018; 29: 1763-70).

The use of trilaciclib with certain tumor types and chemotherapeutic regimes is believed to enhance immune activation and promote antitumor immunity by differentially arresting cytotoxic and regulatory T cell subsets followed by a faster recovery of cytotoxic T lymphocytes (CTLs) compared with regulatory T cells (Tregs) in tumors. This differential alteration of cell cycle kinetics between CTLs and Tregs results in a higher proportion of CTLs to Tregs, the enhancement of T-cell activation, and a decrease in Treg-mediated immunosuppressive functions. Together, these events promote the CTL-mediated clearance of tumor cells. Therefore, the anti-tumor effects of trilaciclib result from the transient proliferative arrest of T cells (protecting them from chemotherapy-induced damage), followed by activation of CTLs in the tumor microenvironment in the context of fewer Tregs.

Additionally, T-cell receptor (TCR) analysis demonstrates that trilaciclib may play an important role in expanding anti-tumor T-cell subsets during treatment. As described further in Example 5 below, patients with small cell lung carcinoma receiving etoposide, carboplatin, and a PD-L1 inhibitor (atezolizumab) (E/P/A) who received trilaciclib had a significantly higher number of expanded T-cell clones following treatment with trilaciclib compared with patients receiving only E/P/A (P=0.01, FIG. 11). In addition, patients that responded in the trilaciclib cohort had more T-cell clonal expansion than patients that received placebo (p=0.001), as well as more clonal expansion than patients that did not respond to trilaciclib (p=0.006). Unlike the placebo, trilaciclib significantly increased the number and fraction of newly expanded clones demonstrating that the addition of trilaciclib to the etoposide, carboplatin, atezolizumab treatment regimen enhances the T-cell mediated anti-tumor response. These data support trilaciclib inducing an immune mediated response.

Importantly, the ability to extend overall survival in certain tumor types can be predicted prior to administration. For example, as described in Example 2 below, a statistically significant improvement in overall survival and progression free survival was observed in patients receiving trilaciclib whose TNBC was classified as C2 IFN-γ Dominant according to the Thorsson et al. Six Class Immune Signature classification system (as defined in Thorsson et el., “The Immune Landscape of Cancer,” supra, incorporated herein by reference and discussed further below) versus those patients with TNBC classified as C2 IFN-γ Dominant who did not receive trilaciclib. As described in Example 3, a similar statistically significant improvement in overall survival and progression free survival was observed in patients who received trilaciclib whose TNBC had high “IFN-γ Signature” and “Expanded Immune Signature” scores according to the Ayers et al. classification system (as defined in Ayers et al., “IFN-γ-related mRNA profile predicts clinical response to PD-1 blockade,” supra, incorporated herein by reference and discussed further below) compared to patients with TNBC having a high “IFN-γ Signature” and “Expanded Immune Signature” score and who did not receive trilaciclib. Furthermore, as described in Example 4, patients with TNBC PD-L1 positive tumors receiving trilaciclib had a significantly longer overall survival than patients with TNBC PD-L1 positive tumors who did not receive trilaciclib.

In addition to the immune-activating effects of transient CDK4/6 inhibition, it has been discovered that the effects are independent of a tumor's CDK4/6-replication dependency (See Tables 6-8 below). For example, while mTNBC is predominantly a functionally CDK4/6-replication independent disease, a subset of patients enrolled in this human clinical trial described below had tumors that were CDK4/6-replication dependent. Based on observations from a preclinical study, whereby palbociclib was administered in combination with carboplatin in an Rb-competent murine model (Roberts et al., Multiple roles of cyclin-dependent kinase 4/6 inhibitors in cancer therapy. J Natl Cancer Inst 2012; 104: 476-87), a risk exists that inducing G1 arrest may reduce the proliferation of tumor cells and negatively affect the efficacy of chemotherapy in CDK4/6-replication dependent tumors. However, preclinical studies of this specific class of CDK4/6 inhibitors administered concurrently with a variety of chemotherapy agents and in multiple CDK4/6 dependent murine models, together with clinical data from this study using established signatures of CDK4/6-replication dependence (see Table 6), does not provide evidence that the short acting CDK4/6 inhibitors described herein negatively impacts the antitumor activity of chemotherapy.

Accordingly, as provided herein, the inclusion of a CDK4/6 inhibitor described herein in combination with a chemotherapeutic that enhances an immune-mediated response, for example but not limited to an ICD-inducing chemotherapeutic, can be used to treat CDK4/6-replication dependent tumors, CDK4/6 replication-independent tumors, or a heterogeneous tumor with both CDK4/6 dependent and independent cells, wherein the tumor is hot, or in alternative embodiments, altered immunosuppressive or altered excluded. Likewise, the inclusion of a CDK4/6 inhibitor described herein in combination with a chemotherapeutic, for example an ICD-inducing chemotherapeutic, can be used to treat CDK4/6-replication dependent tumors, CDK4/6 replication-independent tumors, or a heterogeneous tumor with both CDK4/6 dependent and independent cells, wherein the tumor is immunogenic, for example determined to: be immunogenically hot; have a high Immunoscore, for example an Immunoscore of 14; be C2 “IFN-γ Dominant;” have a high “IFN-γ Signature” or “Expanded Immune Signature” score; be PD-L1 positive; or be immunogenic as determined by any other recognizable assessment known in the art.

Thus, in certain aspects provided herein is a method for selecting a patient population for cancer therapy that includes the administration of a CDK 4/6 inhibitor with chemotherapy in a manner that increases the progression free survival or overall survival of the patient comprising:
(i) determining if the cancer has a surrounding microenvironment that is favorable to immune modulation;
(ii) determining if the chemotherapy regimen is capable of inducing an immune-mediated response, for example immunogenic cell death (ICD), and
(iii) if both (i) and (ii) are yes, administering an effective amount of a CDK4/6 inhibitor selected from Compounds I, II, III, IV, or V, or a pharmaceutically acceptable salt thereof, wherein the CDK4/6 inhibitor is administered prior to the administration of the chemotherapy, or optionally prior to and concurrently with administration of chemotherapy;

wherein the increase in progression free survival and/or overall survival is in comparison to the predicted overall survival based on administration of the chemotherapy alone, either based on literature or otherwise publicly available evidence, a comparative during preclinical or clinical trials, or other means accepted by persons skilled in the field.

In some embodiments, the determination of whether the cancer has a surrounding microenvironment that is favorable to immune modulation comprises assessing whether the cancer microenvironment has a sufficiently high level of major histocompatibility complex class I antigens available to initiate an immune effect. In some embodiments, the determination of whether the cancer has a surrounding microenvironment that is favorable to immune modulation comprises assessing whether the cancer microenvironment has a sufficiently high level of major histocompatibility complex class II antigens available to initiate an immune effect. In some embodiments, the determination of whether the cancer has a surrounding microenvironment that is favorable to immune modulation comprises assessing whether the cancer microenvironment has a sufficiently high level of major histocompatibility complex class I and class II antigens available to initiate an immune effect. In some embodiments, the patient has a cancer that is classified as immunogenic. In some embodiments, the patient has a cancer that is classified as hot, as described herein. In some embodiments, the patient has a cancer that is classified as altered-excluded, as described herein. In some embodiments, the patient has a cancer that is classified as a C2 “IFN-γ Dominant” class cancer, as described herein. In some embodiments, the patient has a cancer that is classified as a high “IFN-γ Signature” or a high “Expanded Immune Signature,” as described herein. In some embodiments, the patient has a cancer that is PD-L1 positive.

In some embodiments, the CDK4/6 inhibitor administered is Compound I, or a pharmaceutically acceptable salt thereof. In some embodiments, the CDK4/6 inhibitor administered is Compound II, or a pharmaceutically acceptable salt thereof. In some embodiments, the CDK4/6 inhibitor administered is Compound III, or a pharmaceutically acceptable salt thereof. In some embodiments, the CDK4/6 inhibitor administered is Compound IV, or a pharmaceutically acceptable salt thereof. In some embodiments, the CDK4/6 inhibitor administered is Compound V, or a pharmaceutically acceptable salt thereof. In some embodiments, the CDK4/6 inhibitor is administered about 24 hours or less prior to the administration of the immune-response mediating chemotherapy, for example, an ICD inducing chemotherapy. In some embodiments, the CDK4/6 inhibitor is administered about 4 hours or less prior to the administration of the immune-response mediating chemotherapy, for example, an ICD inducing chemotherapy. In some embodiments, the CDK4/6 inhibitor is administered about 30 minutes or less prior to administration of the immune-response mediating chemotherapy, for example, an ICD inducing chemotherapy. In some embodiments, the CDK4/6 inhibitor is administered first between about 18 to 28 hours prior to administration of the immune-response mediating chemotherapy, for example, an ICD inducing chemotherapy, and again about 4 hours or less prior to administration of the immune-response mediating chemotherapy, for example, an ICD inducing chemotherapy. In some embodiments, the patient is not administered an immune checkpoint inhibitor. In some embodiments, the CDK4/6 inhibitor is administered one or more times following the completion of chemotherapy treatment in a maintenance treatment regime, for example, once a week, once every two weeks, once every three weeks, once a month, once every six months. In some embodiments, the CDK4/6 inhibitor is administered in combination with the chemotherapeutic one or more times following the completion of treatment in a chemotherapy dose reduced maintenance treatment regime, for example, at least once a week, at least once every two weeks, at least once every three weeks, at least once a month, at least once every six weeks, at least once every two months, at least once every three months, at least once every four months, at least once every five months, or at least once every six months.

In alternative embodiments, A method for selecting a patient population for cancer therapy that includes the administration of a CDK 4/6 inhibitor with chemotherapy in a manner that increases the progression free survival or overall survival of the patient comprising:
(i) determining whether the cancer is immunogenically susceptible to CDK4/6 inhibitor treatment;
(ii) determining whether the patient can be administered a chemotherapy that induces an immune-response, for example an ICD-inducing chemotherapy, based on the cancer;
(iii) and, if it is determined that the cancer is immunogenically susceptible to CDK4/6 inhibitor treatment and that a chemotherapy that induces an immune-response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V, or pharmaceutically acceptable salt thereof, wherein the CDK4/6 inhibitor is administered prior to administration of the chemotherapy, or alternatively, prior to and concurrently with the administration of the chemotherapy and, wherein the improvement in progression free survival and/or overall survival is in comparison to the progression free survival and/or overall survival based on administration of the chemotherapy alone, either based on literature or otherwise publicly available evidence, a comparative during preclinical or clinical trials, or other means accepted by persons skilled in the field.

In some embodiments, the determination of whether the cancer is immunogenically susceptible to CDK4/6 inhibitor treatment comprises assessing whether the cancer microenvironment has a sufficiently high level of major histocompatibility complex class I antigens available to initiate an immune effect. In some embodiments, the determination of whether the cancer is immunogenically susceptible to CDK 4/6 inhibitor treatment comprises assessing whether the cancer microenvironment has a sufficiently high level of major histocompatibility complex class II antigens available to initiate an immune effect. In some embodiments, the determination of whether the cancer is immunogenically susceptible to CDK4/6 inhibitor treatment comprises assessing whether the cancer microenvironment has a sufficiently high level of major histocompatibility complex class I and class II antigens available to initiate an immune effect. In some embodiments, the patient has a cancer that is classified as immunogenic. In some embodiments, the patient has a cancer that is classified as hot, as described herein. In some embodiments, the patient has a cancer that is classified as altered-excluded, as described herein. In some embodiments, the patient has a cancer that is classified as a C2 “IFN-γ Dominant” class cancer, as described herein. In some embodiments, the patient has a cancer that is classified as a high “IFN-γ Signature” or a high “Expanded Immune Signature,” as described herein. In some embodiments, the patient has a cancer that is PD-L1 positive.

In some embodiments, the CDK4/6 inhibitor administered is Compound I, or a pharmaceutically acceptable salt thereof. In some embodiments, the CDK4/6 inhibitor administered is Compound II, or a pharmaceutically acceptable salt thereof. In some embodiments, the CDK4/6 inhibitor administered is Compound III, or a pharmaceutically acceptable salt thereof. In some embodiments, the CDK4/6 inhibitor administered is Compound IV, or a pharmaceutically acceptable salt thereof. In some embodiments, the CDK4/6 inhibitor administered is Compound V, or a pharmaceutically acceptable salt thereof. In some embodiments, the CDK4/6 inhibitor is administered about 24 hours or less prior to the administration of the immune-response mediating chemotherapy, for example, an ICD inducing chemotherapy. In some embodiments, the CDK4/6 inhibitor is administered about 4 hours or less prior to the administration of the immune-response mediating chemotherapy, for example, an ICD inducing chemotherapy. In some embodiments, the CDK4/6 inhibitor is administered about 30 minutes or less prior to administration of the immune-response mediating chemotherapy, for example, an ICD-inducing chemotherapy. In some embodiments, the CDK4/6 inhibitor is administered first about 22 to 26 hours prior to administration of the immune-response mediating chemotherapy, for example, an ICD-inducing chemotherapy, and again about 4 hours or less prior to administration of the immune-response mediating chemotherapy, for example, an ICD-inducing chemotherapy. In some embodiments, the patient is not administered an immune checkpoint inhibitor. In some embodiments, the CDK4/6 inhibitor is administered one or more times following the completion of chemotherapy treatment in a maintenance treatment regime, for example, once a week, once every two weeks, once every three weeks, once a month, once every six months. In some embodiments, the CDK4/6 inhibitor is administered in combination with the chemotherapeutic one or more times following the completion of treatment in a chemotherapy dose reduced maintenance treatment regime, for example, at least once per week, at least once every two weeks, at least once every three weeks, at least once a month, at least once every two months, at least once every six weeks, at least once every three months, at least once every four months, at least once every five months, or at least once every six months.

Chemotherapies capable of inducing an immune-mediated responses are generally known in the art and include, but are not limited to, alkylating agents such as cyclophosphamide, trabectedin, temozolomide, melphalan, dacarbazine, and oxaliplatin; antimetabolites such as methotrexate, mitoxantrone, gemcitabine, and 5-fluorouracil (5-FU); cytotoxic antibiotics such as bleomycin and anthracyclines, including doxorubicin, daunorubicin, epirubicin, idarubicin, and valrubicin; taxanes, such as paclitaxel cabazitaxel, and docetaxel; topoisomerase inhibitors such as topotecan, irinotecan, and etoposide; platinum compounds such as carboplatin and cisplatin; bortezomib, an inhibitor of the 26S proteasome subunit; Vinca alkaloids such as vinblastine, vincristine, vindesine, and vinorelbine; diaziquone; mechlorethamine; mitomycin C; fludarabine; cytosine arabinoside; and combinations of any thereof. In some embodiments, the ICD-inducing chemotherapy is selected from idarubicin, epirubicin, doxorubicin, mitoxantrone, oxaliplatin, bortezomib, gemcitabine, and cyclophosphamide, and combinations thereof.

Methods for determining whether a patient with a particular cancer is a candidate to receive a chemotherapy capable of inducing an immune response is known, however, the effect of a CDK 4/6 inhibitor on such therapy has not been fully explored, especially without an immune checkpoint inhibitor. Considerations include whether the type of cancer to be treated is known to be responsive to the particular chemotherapeutic agent, whether the patient has received the prior chemotherapeutic agent in the past, and whether the patient's cancer has developed a resistance to the chemotherapy or has a phenological characteristic rendering the chemotherapy ineffective.

The targeted cancers suitable for the treatment using the presently described methods with a CDK 4/6 inhibitor include those tumors that are immunogenic or susceptible to an immuno-oncology chemotherapeutic treatment regimen. In some embodiments, the patient to be treated has an immunogenic cancer selected from the group consisting of breast cancer, including estrogen receptor (ER)-positive breast cancer, triple negative breast cancer, non-small cell lung carcinoma, head and neck squamous cell cancer, classical Hodgkin lymphoma (cHL), bladder cancer, primary mediastinal B-cell lymphoma (PBMCL), diffuse large B-cell lymphoma, urothelial carcinoma, microsatellite instability-high (MSI—H) solid tumors, mismatch repair deficient (dMMR) solid tumors, gastric or gastroesophageal junction (GEJ) adenocarcinoma, squamous cell carcinoma of the esophagus, cervical cancer, endometrial cancer, cholangiocarcinoma, hepatocellular carcinoma, Merkel cell carcinoma, renal cell carcinoma, ovarian cancer, anal canal cancer, colorectal cancer, skin cutaneous melanoma, endometrial cancer, and melanoma.

Accordingly, methods provided herein include:

A. A method for selecting a patient or patient population for cancer therapy that includes the administration of a CDK 4/6 inhibitor with chemotherapy in a manner that increases the progression free survival or overall survival of the patient comprising: (i) determining if the cancer has a surrounding microenvironment that is favorable to immune modulation; (ii) determining whether the chemotherapy regimen induces a immune-mediated response such as immunogenic cell death, and (iii) if both (i) and (ii) are yes, administering an effective amount of a CDK4/6 inhibitor selected from Compounds I, II, III, IV, or V, or a pharmaceutically acceptable salt thereof, wherein the CDK4/6 inhibitor is administered prior to the administration of the chemotherapy or optionally prior to and concurrently with chemotherapy; and, wherein the increase in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

B. A method for selecting a patient or patient population for cancer therapy that includes the administration of a CDK 4/6 inhibitor with chemotherapy in a manner that increases the progression free survival or overall survival of the patient comprising: (i) determining the immunogenic classification of the cancer; (ii) determining whether the patient can be administered a chemotherapy capable of inducing an immune-mediated response, for example an ICD-inducing chemotherapy, based on the cancer; and, (iii) if it is determined a chemotherapy capable of inducing an immune-mediated response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V or pharmaceutically acceptable salt thereof, wherein the CDK4/6 inhibitor is administered prior to administration of the chemotherapy or optionally prior to and concurrently with chemotherapy, and wherein the improvement in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

C. A method for selecting a patient or patient population for cancer therapy that includes the administration of a CDK 4/6 inhibitor with chemotherapy in a manner that increases the progression free survival or overall survival of the patient comprising: (i) determining whether the cancer is immunogenically susceptible to CDK4/6 inhibitor treatment; (ii) determining whether the patient can be administered a chemotherapy that induces an immune-response, for example an ICD-inducing chemotherapy, based on the cancer; (iii) and, if it is determined that the cancer is immunogenically susceptible to CDK4/6 inhibitor treatment and that a chemotherapy that induces an immune-response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V, or pharmaceutically acceptable salt thereof, wherein the CDK4/6 inhibitor is administered prior to administration of the chemotherapy, or alternatively, prior to and concurrently with the administration of the chemotherapy, and, wherein the improvement in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

D. A method for selecting a patient or patient population for cancer therapy that includes the administration of a CDK 4/6 inhibitor with chemotherapy in a manner that increases the progression free survival or overall survival of the patient comprising: (i) determining whether the cancer is immunogenic; (ii) determining whether the patient can be administered a chemotherapy that induces an immune-response, for example an ICD-inducing chemotherapy, based on the cancer; (iii) and, if it is determined that the cancer is immunogenic and that a chemotherapy that induces an immune-response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V, or pharmaceutically acceptable salt thereof, wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

E. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising: (i) determining if the cancer has a surrounding microenvironment that is favorable to immune modulation; (ii) determining if the chemotherapy regimen induces an immune-mediated response such as immunogenic cell death, and (iii) if both (i) and (ii) are yes, administering an effective amount of a CDK4/6 inhibitor selected from Compounds I, II, III, IV, or V, or a pharmaceutically acceptable salt thereof, wherein the CDK4/6 inhibitor is administered prior to the administration of the chemotherapy or optionally prior to and concurrently with chemotherapy; and, wherein the increase in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

F. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising; (ii) determining whether the patient can be administered a chemotherapy capable of inducing an immune-mediated response, for example an ICD-inducing chemotherapy, based on the cancer; and, (iii) if it is determined a chemotherapy capable of inducing an immune-mediated response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V or pharmaceutically acceptable salt thereof, wherein the CDK4/6 inhibitor is administered prior to administration of the chemotherapy or optionally prior to and concurrently with chemotherapy, and wherein the improvement in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

G. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising: (i) determining whether the cancer is immunogenically susceptible to CDK4/6 inhibitor treatment; (ii) determining whether the patient can be administered a chemotherapy that induces an immune-response, for example an ICD-inducing chemotherapy, based on the cancer; (iii) and, if it is determined that the cancer is immunogenically susceptible to CDK 4/6 inhibitor treatment and that a chemotherapy that induces an immune-response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V, or pharmaceutically acceptable salt thereof, wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

H. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising: (i) determining whether the cancer is immunogenic; (ii) determining whether the patient can be administered a chemotherapy that induces an immune-response, for example an ICD-inducing chemotherapy, based on the cancer; (iii) and, if it is determined that the cancer is immunogenic and that a chemotherapy that induces an immune-response, for example an ICD-inducing chemotherapy, can be administered, administering an effective amount of the chemotherapy in combination with an effective amount of a short-acting CDK4/6 inhibitor selected from Compound I, Compound II, Compound III, Compound IV, or Compound V, or pharmaceutically acceptable salt thereof, wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone, either based on literature or otherwise publicly available evidence, a comparative during preclinical or clinical trials, or other means accepted by persons skilled in the field. In some embodiments, the patient is not administered a check-point inhibitor in during the treatment regimen.

### BRIEF DESCRIPTION OF THE FIGURES

FIG. 1 is a study schematic of G1T28-04 human clinical trial assessing the clinical benefit of trilaciclib (Compound I) in preserving the bone marrow and the immune system, and enhancing chemotherapy antitumor efficacy when administered prior to carboplatin and gemcitabine (GC therapy) for patients with metastatic triple negative breast cancer (mTNBC). The treatment phase consisted of 21-day cycles: Trilaciclib was administered intravenously prior to gemcitabine/carboplatin infusions at 240 mg/m². Gemcitabine was administered via IV at 1000 mg/m². Carboplatin was administered IV at a calculated dose based on an area under the curve (AUC) of 2 for each patient. Peripheral blood samples were collected predose and on Day 1 of odd cycles; at the post treatment visit; and at first Survival follow up for flow cytometric analysis. Tumor assessments were completed every 9 weeks through Week 39 and every 12 weeks thereafter. As provided in the figure: LOT=Lines of Therapy, Trila=Trilaciclib, Tox=Toxicity, PD=Disease Progression, WD=Withdrew, DC=discontinued, PI=Principal Investigator, ANC=absolute neutrophil count, IV=Intravenous, OS=Overall Survival, PTV=Post Treatment Visit, FU=Follow U. Trilaciclib's effects on PFS and OS have been stabilized between two data snapshots: data cut-off date Jun. 28, 2019 and almost a year later, May 15, 2020.

FIG. 2 is a Kaplan-Meier plot of overall survival of triple negative breast cancer human patients from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle). The x-axis depicts months from randomization and number of patients at risk. The y-axis depicts the probability of being alive. Overall survival was significantly longer for Group 3 vs. Group 1 (Hazard Ratio=0.40; nominal p=0.0004) and Group 2 v. Group 1 (Hazard Ratio=0.31; nominal p=0.0016). Data cut-off date May 15, 2020.

FIG. 3 is a Kaplan-Meier plot of progression free survival of triple negative breast cancer human patients from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle). The x-axis depicts months from randomization and number of patients at risk. The y-axis depicts the probability of being progression free. Data cut-off date May 15, 2020.

FIG. 4A is a Forest plot of overall survival in triple negative breast cancer human patients from Group 1 (gemcitabine+carboplatin only on days 1 and 8 in a 21-day cycles), Group 2 (gemcitabine+carboplatin+trilaciclib on days 1 and 8 in a 21-day cycle), and Group 3 (gemcitabine+carboplatin on days 2 and 9+trilaciclib on days 1, 2, 8, and 9 in a 21-day cycle). Data are from the intention to treat population and data from trilaciclib groups 2 and 3 were pooled for prespecified subgroup analysis. Acquired triple-negative breast cancer refers to a patient with confirmed metastatic triple negative breast cancer and any previous biopsy showing estrogen and progesterone receptor or HER2 positivity. Data from trilaciclib Groups 2 and 3 were pooled for prespecified subgroup analysis. The two-sided p-value was obtained from the stratified log-rank test. The Hazard Ratio between the two treatment groups (trilaciclib vs gemcitabine/carboplatin only), together with its 95% confidence intervals (CIs), were calculated from a Cox proportional hazard model in which treatment and the applicable stratification factors were included as fixed terms. As provided in the plot: ECOG=Eastern Cooperative Oncology Group. P-values were obtained from stratified log-rank test with applicable stratification factors as covariates. Analysis performed with data cut-off date of Jun. 28, 2019.

FIG. 4B is a Forest plot of progression free survival in triple negative breast cancer human patients from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle). Data are from the intention to treat population and data from trilaciclib groups 2 and 3 were pooled for prespecified subgroup analysis. Acquired triple-negative breast cancer refers to a patient with confirmed metastatic triple negative breast cancer and any previous biopsy showing estrogen and progesterone receptor or HER2 positivity. Data from trilaciclib Groups 2 and 3 were pooled for prespecified subgroup analysis. The two-sided p-value was obtained from the stratified log-rank test. The HR (Hazard Ratio) between the two treatment groups (trilaciclib vs gemcitabine/carboplatin only), together with its 95% confidence intervals (CIs), were calculated from a Cox proportional hazard model in which treatment and the applicable stratification factors were included as fixed terms. As provided in the plot: ECOG=Eastern Cooperative Oncology Group. P-values were obtained from stratified log-rank test with applicable stratification factors as covariates. Analysis performed with Data cut-off date of Jun. 28, 2019.

FIG. 4C is a Forest plot of overall survival in triple negative breast cancer human patients from Group 1 (gemcitabine+carboplatin only on days 1 and 8 in a 21-day cycles), Group 2 (gemcitabine+carboplatin+trilaciclib on days 1 and 8 in a 21-day cycle), and Group 3 (gemcitabine+carboplatin on days 2 and 9+trilaciclib on days 1, 2, 8, and 9 in a 21-day cycle). Data are from the intention to treat population and data from trilaciclib groups 2 and 3 were pooled for prespecified subgroup analysis. Acquired triple-negative breast cancer refers to a patient with confirmed metastatic triple negative breast cancer and any previous biopsy showing estrogen and progesterone receptor or HER2 positivity. Data from trilaciclib Groups 2 and 3 were pooled for prespecified subgroup analysis. The two-sided p-value was obtained from the stratified log-rank test. The Hazard Ratio between the two treatment groups (trilaciclib vs gemcitabine/carboplatin only), together with its 95% confidence intervals (CIs), were calculated from a Cox proportional hazard model in which treatment and the applicable stratification factors were included as fixed terms. As provided in the plot: ECOG=Eastern Cooperative Oncology Group. P-values were obtained from stratified log-rank test with applicable stratification factors as covariates. Analysis performed with data cut-off date of May 15, 2020.

FIG. 4D is a Forest plot of progression free survival in triple negative breast cancer human patients from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle). Data are from the intention to treat population and data from trilaciclib groups 2 and 3 were pooled for prespecified subgroup analysis. Acquired triple-negative breast cancer refers to a patient with confirmed metastatic triple negative breast cancer and any previous biopsy showing estrogen and progesterone receptor or HER2 positivity. Data from trilaciclib Groups 2 and 3 were pooled for prespecified subgroup analysis. The two-sided p-value was obtained from the stratified log-rank test. The HR (Hazard Ratio) between the two treatment groups (trilaciclib vs gemcitabine/carboplatin only), together with its 95% confidence intervals (CIs), were calculated from a Cox proportional hazard model in which treatment and the applicable stratification factors were included as fixed terms. As provided in the plot: ECOG=Eastern Cooperative Oncology Group. P-values were obtained from stratified log-rank test with applicable stratification factors as covariates. Analysis performed with Data cut-off date of May 15, 2020.

FIG. 5 is a graph showing the normalized mean frequency of interferon-gamma (IFN-γ)+ population of CD8+ T cells after ex vivo stimulation (IFN-γ+IL-17A-[CD3+CD8+]). Analyses included only patients (n=8-15 per cohort) who received three or more cycles of gemcitabine/carboplatin (GC) with statistical outliers excluded. Error bars represent 95% confidence intervals. Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle). Data points represent samples taken from the same patient at different timepoints. As provided in the graph: C=cycle; D=day.

FIG. 6 is a reproduction of FIG. 3 found in Galon, J., and Bruni, D., Approaches to treat immune hot, altered and cold tumours with combination immunotherapies, Nature Reviews Drug Discovery (18), March 2019, 197-218, incorporated herein in its entirety, describing an immunogram for use as a tool to direct anticancer therapy. Cancers can be classified into four main subtypes (hot, altered-excluded, altered-immunosuppressed and cold) according to their associated T cell (CD3+ and CD8+) presence and distribution. Hot cancers are defined by the simultaneous presence of immune contexture parameters: the cell type (CD3+, CD8+, follicular helper T (TFH), T helper 1 (TH1), memory and exhausted T cells); the location (invasive margin, tumor core and tertiary lymphoid structures); the density (immune density and quantity); and the functional immune orientation (chemokines, cytokines, cytotoxic factors, adhesion, attraction and TH1). As provided in the immunogram: DORA2A, A2A adenosine receptor; β m, β2-microglobulin; BET, bromodomain and extra-terminal motif proteins; BTLA, B and T lymphocyte attenuator; CAR T-cell, chimeric antigen receptor T-cell; CCR, CC-chemokine receptor; CIN, chromosomal instability, CSF1R, colony-stimulating factor 1 receptor; CTL A4, cytotoxic T lymphocyte-associated antigen; CXCL, CXC-chemokine ligand; DDR, DNA damage response; ECM, extracellular matrix; EMT, epithelial-mesenchymal transition; FDA, US Food and Drug Administration; FGFR3, fibroblast growth factor receptor 3; FOXP3, forkhead box P3; GITR, glucocorticoid-induced TNFR-related protein; GM-CSF, granulocyte-macrophage colony-stimulating factor; HDAC, histone deacetylase; HIF1α, hypoxia-inducible factor 1-α; HLA, human leukocyte antigen; HMA, hypomethylating agent; IAP, inhibitors of apoptosis family (also known as XIAP); ICAM1, intercellular adhesion molecule 1; ICD, immunogenic cell death; ICOS, inducible T-cell co-stimulator; ICP, immune checkpoint; IDO, indoleamine 2,3-dioxygenase; IFN, interferon; IL, interleukin; LAG3, lymphocyte activation gene 3; LIGHT, tumor necrosis factor superfamily member 14; MAdCAM1, mucosal addressin cell adhesion molecule 1; MCL1, induced myeloid leukemia cell differentiation protein Mcl1; MDSCs, myeloid-derived suppressor cells; MEK, mitogen-activated protein kinase; MET, mesenchymal-epithelial transition; MSI, microsatellite instability; NK, natural killer; NOS1, nitric oxide synthase 1; PD-1, programmed cell death protein 1; PD-L1, PD-1 ligand; PI3Kγ, phosphoinositide 3-kinase-γ; PPARγ, peroxisome proliferator-activated receptor-γ; SIGLEC9, sialic acid-binding Ig-like lectin 9; STING, stimulator of interferon genes; TDO, tryptophan 2,3-dioxygenase; TGFβ, transforming growth factor-β; TIGIT, T-cell immunoglobulin and ITIM domain; TIM3, T-cell immunoglobulin and mucin domain-containing 3; TKI, tyrosine kinase inhibitor; TLR, Toll-like receptor; Treg cells, regulatory T-cells; VCAM1, vascular cell adhesion molecule 1; VEGF, vascular endothelial growth factor; VISTA, V-domain Ig suppressor of T-cell activation; XCL1, lymphotactin; XCR1, chemokine XC receptor 1. Lower case “i” following any acronym or abbreviation indicates inhibitor; lower case “a” following any acronym or abbreviation indicates agonist.

FIG. 7A is a reproduction of FIG. 1A found in Galon, J., and Bruni, D., Approaches to treat immune hot, altered and cold tumors with combination immunotherapies, Nature Reviews Drug Discovery (18), March 2019, 197-218, incorporated herein in its entirety, which illustrates examples of hot, altered and cold immune cancers. Dark (3,3′-diaminobenzidine (DAB)) staining represents CD3+ T cells and lighter (alkaline phosphatase) counterstaining provides homogeneous tissue background staining. The level and spatial distribution of CD3+ and CD8+ T cell infiltration differentiates four distinct solid tumor phenotypes: hot (or inflamed); altered, which can be excluded or immunosuppressed; and cold (or non-inflamed). These tumor phenotypes are characterized by high, intermediate, and low immunoscore, respectively.

FIG. 7B is a reproduction of FIG. 1B found in Galon, J., and Bruni, D., Approaches to treat immune hot, altered and cold tumors with combination immunotherapies, Nature Reviews Drug Discovery (18), March 2019, 197-218, incorporated herein in its entirety, which is a schematic representation of the four subtypes of immune tumor. Of note, in altered-excluded tumors, CD3+ and CD8+ T cell infiltrates are low at the tumor center and high at the invasive margin, resulting overall in an intermediate immunoscore. Altered-immunosuppressed tumors display instead a more uniform pattern of (low) CD3+ and CD8+ T cell infiltration. CT, center of tumor; Hi, high; IM, invasive margin; Lo, low.

FIG. 8A is a schematic of the distribution of Ayers' IFN-γ Signature scores from pre-treatment cancer samples from patients participating in the G1T28-04 clinical trial (NCT02978716). The x-axis represents the probability density curve, and the y-axis is the calculated Ayers' IFN-γ Signature score. The vertical dash line on the left side of the graph represents tertile 1, the vertical dash line on the right side of the graph represents tertile 2, and the vertical dash line between tertile 1 and tertile 2 represents the median.

FIG. 8B is a schematic of the distribution of Ayers' Expanded Immune Signature scores from pre-treatment cancer samples from patients participating in the G1T28-04 clinical trial (NCT02978716). The x-axis represents the probability density curve, and the y-axis is the calculated Ayers' IFN-γ Signature score. The vertical dash line on the left side of the graph represents tertile 1, the vertical dash line on the right side of the graph represents tertile 2, and the vertical dash line between tertile 1 and tertile 2 represents the median.

FIG. 8C is a Kaplan-Meier plot of overall survival of triple negative breast cancer human patients in G1T28-04 clinical trial (NCT02978716) from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a high Ayers' IFN-γ Signature score. The x-axis depicts months from randomization. The y-axis depicts the probability of being alive. Overall survival was significantly longer for Group 4 (Groups 2+3) vs. Group 1 (p=0.0194). Legend:______ Group 1; ______Group 2; ______Group 3; ______Group 4.

FIG. 8D is a Kaplan-Meier plot of overall survival of triple negative breast cancer human patients in G1T28-04 clinical trial (NCT02978716) from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a low Ayers' IFN-γ Signature score. The x-axis depicts months from randomization. The y-axis depicts the probability of being alive. Legend:______ Group 1; ______Group 2; ______Group 3; ______Group 4.

FIG. 8E is a Kaplan-Meier plot of progression free survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a low Ayers' IFN-γ Signature score. The x-axis depicts months from randomization. The y-axis depicts the probability of being alive. Legend:______ Group 1; ______Group 2; ______Group 3; ______Group 4.

FIG. 8F is a Kaplan-Meier plot of progression free survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a low Ayers' IFN-γ Signature score. The x-axis depicts months from randomization. The y-axis depicts the probability of being alive. Legend:______ Group 1; ______Group 2; ______Group 3; ______Group 4.

FIG. 9A is a Kaplan-Meier plot of overall survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a high Ayers' Expanded Immune Signature score. The x-axis depicts months from randomization. The y-axis depicts the probability of being alive. Overall survival was significantly longer for Group 4 (Groups 2+3) vs. Group 1 (p=0.0266). Legend:______ Group 1; ______Group 2; ______Group 3; ______Group 4.

FIG. 9B is a Kaplan-Meier plot of overall survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a low Ayers' Expanded Immune Signature score. The x-axis depicts months from randomization. The y-axis depicts the probability of being alive. Legend:______ Group 1; ______Group 2; ______Group 3; ______Group 4.

FIG. 9C is a Kaplan-Meier plot of progression free survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a high Ayers' Expanded Immune Signature score. The x-axis depicts months from randomization. The y-axis depicts the probability of being alive. Legend:______ Group 1; ______Group 2; ______Group 3; ______Group 4.

FIG. 9D is a Kaplan-Meier plot of progression free survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a low Ayers' Expanded Immune Signature score. The x-axis depicts months from randomization. The y-axis depicts the probability of being alive Legend:______ Group 1; ______Group 2; ______Group 3; ______Group 4.

FIG. 10A is a Kaplan-Meier plot of overall survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a C2 IFN-γ Dominant Six Class Immune Signature score. The x-axis depicts months from randomization. The y-axis depicts the probability of being alive. Overall survival was significantly longer for Group 4 (Groups 2+3) vs. Group 1 (p=0.036 Legend:______ Group 1; ______Group 2; ______Group 3; ______Group 4.

FIG. 10B is a Kaplan-Meier plot of overall survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a non-C2 IFN-γ Dominant Six Class Immune Signature score. The x-axis depicts months from randomization. The y-axis depicts the probability of being alive. Legend:______ Group 1; ______Group 2; ______Group 3; ______Group 4.

FIG. 10C is a Kaplan-Meier plot of progression free survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have C2 IFN-γ Dominant Six Class Immune Signature score. The x-axis depicts months from randomization. The y-axis depicts the probability of being alive. Legend:______ Group 1; ______Group 2; ______Group 3; ______Group 4.

FIG. 10D is a Kaplan-Meier plot of progression free survival of triple negative breast cancer human patients in G1T28-04 clinical trial from Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and Group 4 (Group 2+Group 3) determined to have a non-C2 IFN-γ Dominant Six Class Immune Signature score. The x-axis depicts months from randomization. The y-axis depicts the probability of being alive. Legend:______ Group 1; ______Group 2; ______Group 3; ______Group 4.

FIG. 11 is a schematic representing the number of expanded T-cell clones determined by the differential abundance analysis of T-cell receptor β sequences in whole blood from patients receiving trilaciclib or placebo at after induction and prior to starting maintenance versus baseline (prior to induction). Horizontal bars indicate median number of expanded clones in each group.

FIG. 12 is a schematic representing the number of expanded T-cell clones determined by the differential abundance analysis of T-cell receptor β sequences in whole blood from responders and non-responders at after induction and prior to starting maintenance versus baseline (prior to induction). Horizontal bars indicate median number of expanded clones in each group.

FIG. 13 is a schematic representing the number of newly expanded T-cell clones in responders and non-responders receiving placebo or trilaciclib at after induction and prior to starting maintenance versus baseline (prior to induction). Horizontal bars indicate median number of expanded clones in each group.

FIG. 14 is a is a schematic representing the fraction of newly expanded T-cell clones in responders and non-responders receiving placebo or trilaciclib at after induction and prior to starting maintenance versus baseline (prior to induction). Horizontal bars indicate median number of expanded clones in each group.

### DETAILED DESCRIPTION OF THE INVENTION

### Definitions

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individual recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and do not pose a limitation on the scope of the invention unless otherwise claimed. Unless defined otherwise, technical and scientific terms used herein have the same meaning as is commonly understood by one of skill in the art to which this invention belongs.

An “effective amount” as used herein, means an amount which provides a therapeutic or prophylactic benefit.

To “treat” a disease as the term is used herein, means to reduce the frequency or severity of at least one sign or symptom of a disease or disorder experienced by a patient (i.e. palliative treatment) or to decrease a cause or effect of the disease or disorder (i.e. disease-modifying treatment).

As provided herein, the “host,” “subject,” “patient,” or “individual” to be treated according to the methods described herein is a mammal, including a human.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active compound is provided.

As used herein, the term “immune checkpoint inhibitor (ICI)” refers to inhibitory therapy targeting immune checkpoints, key regulators of the immune system that when stimulated can dampen the immune response to an immunologic stimulus. Some cancers can protect themselves from attack by stimulating immune checkpoint targets. ICIs block inhibitory checkpoints, restoring immune system function. ICIs include those targeting immune checkpoint proteins such as programmed cell death-1 protein (PD-1), PD-1 Ligand-1 (PD-L1), PD-1 Ligand-2 (PD-L2), CTLA-4, LAG-3, TIM-3, and V-domain Ig suppressor of T-cell activation (VISTA), B7-H3/CD276, indoleamine 2,3-dioxygenase (IDO), killer immunoglobulin-like receptors (KIRs), carcinoembryonic antigen cell adhesion molecules (CEACAM) such as CEACAM-1, CEACAM-3, and CEACAM-5, sialic acid-binding immunoglobulin-like lectin 15 (Siglec-15), T cell immunoreceptor with Ig and ITIM domains (TIGIT), and B and T lymphocyte attenuator (BTLA) protein. Immune checkpoint inhibitors are known in the art.

In some embodiments, the term “CDK4/6-replication independent cancer” refers to a cancer that does not significantly require the activity of CDK4/6 for replication. Cancers of such type are often, but not always, characterized by (e.g., that has cells that exhibit) an increased level of CDK2 activity or by reduced expression of retinoblastoma tumor suppressor protein or retinoblastoma family member protein(s), such as, but not limited to p107 and p130. The increased level of CDK2 activity or reduced or deficient expression of retinoblastoma tumor suppressor protein or retinoblastoma family member protein(s) can be increased or reduced, for example, compared to normal cells. In some embodiments, the increased level of CDK2 activity can be associated with (e.g., can result from or be observed along with) MYC proto-oncogene amplification or overexpression. In some embodiments, the increased level of CDK2 activity can be associated with overexpression of Cyclin E1, Cyclin E2, or Cyclin A.

In some embodiments, the term “CDK4/6-replication dependent cancer” refers to a cancer that requires the activity of CDK4/6 for replication or proliferation, or which may be growth inhibited through the activity of a selective CDK4/6 inhibitor. Cancers and disorders of such type may be characterized by (e.g., that has cells that exhibit) the presence of a functional Retinoblastoma (Rb) protein. Such cancers and disorders are classified as being Rb-positive.

Predicting Anti-Cancer/Immunological Effect of CDK4/6 Inhibitor Therapy

Multiple tumor-related or immune-related biomarkers may be used as a predictor of whether an anti-tumor effect can be realized with the addition of a CDK4/6 inhibitor to a chemotherapeutic regime, including an ICD-inducing chemotherapy. Such predictive biomarkers include the expression of immunosuppressive molecules (such as PD-L1) by tumor cells; the molecular profiling of the tumor microenvironment, which encompasses the expression of inflammatory genes; the assessment of the mutational landscape and neoantigen load; mismatch-repair deficiency and MSI; tumor aneuploidy; immune infiltration; and immunoscore (see generally Galon, J., and Bruni, D., Approaches to treat immune hot, altered and cold tumors with combination immunotherapies, Nature Reviews Drug Discovery (18), March 2019, 197-218, incorporated by reference herein). For example, tumors with high levels of somatic copy-number alteration (SCNA) are associated with reduced expression of cytotoxic immune infiltration in patients with melanoma (see Davoli et al., Tumor aneuploidy correlates with markers of immune evasion and with reduced response to immunotherapy, Science 355, eaaM99 (2017)). Patients with tumors having high levels of SCNA and reduced expression of cytotoxic immune infiltration may not be predicted to realize an anti-tumor effect resulting in an increased overall survival with the addition of a CDK4/6 inhibitor to their chemotherapeutic regime.

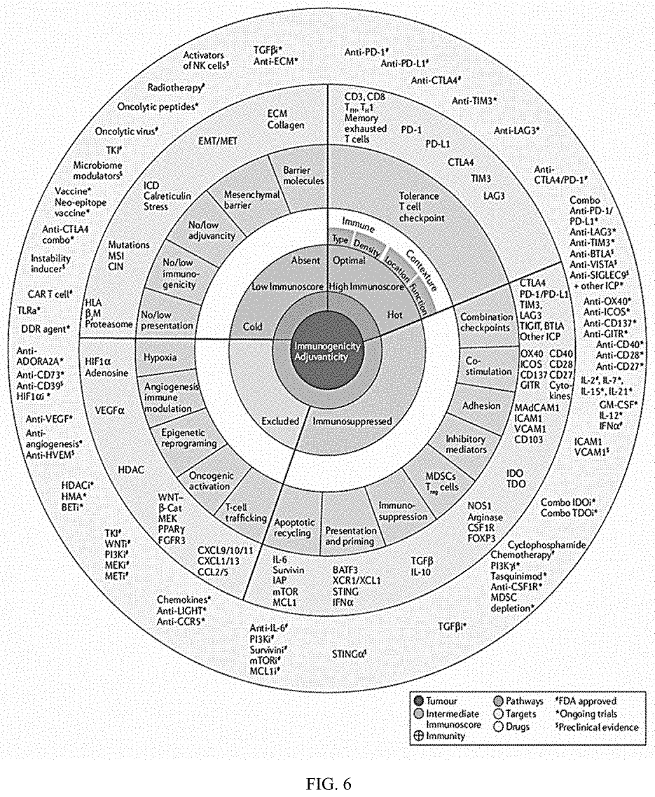

Importantly, while Galon, J., and Bruni, D., Approaches to treat immune hot, altered and cold tumours with combination immunotherapies, supra describe a large number of strategies in treating tumors in a way to induce improved immune responses, it does not describe the use of a CDK4/6 inhibitor in combination with an ICD-inducing chemotherapy to do so (see FIG. 6). The Galon article highlights the inventive and surprising aspect of the present invention because, despite the comprehensiveness of the article in a prestigious scientific journal, it did not mention or suggest the judicious use or the appropriate selection of a protocol that includes a CDK4/6 inhibitor in the immunogram as part of an effective anticancer therapy that can increase progression free survival or overall survival in a cancer patient. This clinical result is surprising. Additional parameters including tumor foreignness, general immune status, immune cell infiltration, absence of checkpoints, absence of soluble inhibitors, absence of inhibitory tumor metabolism, and tumor sensitivity to immune effectors can be used to determine whether an anti-tumor effect resulting in an increased overall survival can be realize with the addition of a CDK4/6 inhibitor to their chemotherapeutic regime. The evaluation of these factors can be achieved by a combination of tumor genomics, immunoscore assay, immunohistochemistry, standard blood assays and immune gene signature, both pre-therapy and post-therapy (see Blank et al., The “cancer immunogram,” Science 352, 658-660 (2016), incorporated herein by reference).

Immunogenic Classification of Tumors

As described above, tumors can be classified based on certain immunogenic characteristics. Importantly, it has been observed that a tumor may progress over time through the various classifications. Also, it has been observed that, in certain instances, a type of tumor may have different immunogenic characteristics in different individuals.

As provided herein, hot immune tumors are those that have (i) a high degree of T-cell and cytotoxic T cell infiltration, i.e., a high immunoscore; and (ii) ability for checkpoint activation (programmed cell death protein 1 (PD-1), cytotoxic T lymphocyte-associated antigen 4 (CTLA4), T-cell immunoglobulin mucin receptor 3 (TIM3) and lymphocyte activation gene 3 (LAG3)) or otherwise impaired T-cell functions (for example, extracellular potassium-driven T-cell suppression). In addition to presence of tumor-infiltrating lymphocytes (TILs) and the expression of anti-programmed death-ligand 1 (PD-L1) on tumor-associated immune cells, hot tumors characteristically display possible genomic instability and the presence of a pre-existing antitumor immune response. See, e.g., in Galon, J., and Bruni, D., Approaches to treat immune hot, altered and cold tumors with combination immunotherapies, Nature Reviews Drug Discovery (18), March 2019, 197-218, incorporated herein in its entirety.

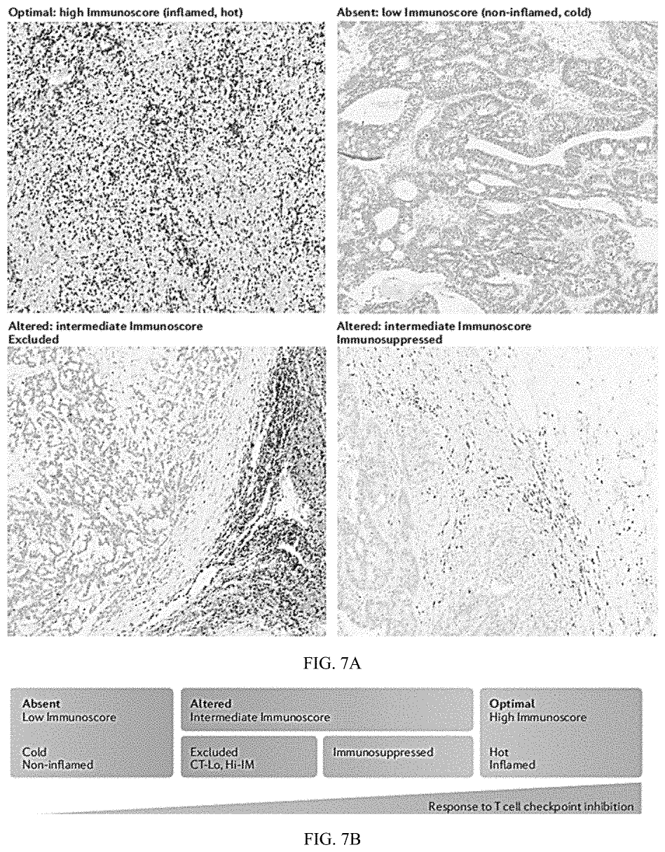

Tumors often observed to have characteristics of hot immune tumors include, but are not limited to, bladder cancers, renal cell carcinoma, liver cancer (hepatocellular carcinoma), non-small cell lung cancer, colon adenocarcinoma, breast invasive carcinoma, cholangiocarcinoma, esophageal carcinoma, Merkel cell carcinoma, HPV+ head and neck squamous cell carcinomas, advanced-stage melanoma, skin cutaneous melanoma, endometrial cancer, gastric cancer and cervical cancer; Hodgkin lymphoma, diffuse large B-cell lymphoma; and tumors with microsatellite instability (MSI). An exemplified resected tumor having the characteristics of a hot immune tumor is illustrated in FIG. 7A.

Altered-immunosuppressed tumors are categorized by (i) poor, albeit not absent, T-cell and cytotoxic T-cell infiltration (intermediate immunoscore), (ii) presence of soluble inhibitory mediators (transforming growth factor-β (TGFβ), interleukin 10 (IL-10) and vascular endothelial growth factor (VEGF)), (iii) the presence of immune suppressive cells (myeloid-derived suppressor cells and regulatory T-cells), and (iv) presence of T-cell checkpoints (PD-1, CTLA4, TIM3 and LAG3). Altered-immunosuppressed tumor sites display a low degree of immune infiltration (FIG. 7A), which suggests the absence of physical barriers and the presence of an immuno-suppressive environment that limits further T-cell recruitment and expansion. See, e.g., in Galon, J., and Bruni, D., Approaches to treat immune hot, altered and cold tumors with combination immunotherapies, Nature Reviews Drug Discovery (18), March 2019, 197-218, incorporated herein in its entirety. An exemplified resected tumor having the characteristics of an altered-immunosuppressed immune tumor is illustrated in FIG. 7A.

The characteristics of altered-excluded immune tumors are (i) no T-cell infiltration inside the tumor bed; accumulation of T-cells at tumor borders (invasive margin) (intermediate immunoscore), (ii) activation of oncogenic pathways, (iii) epigenetic regulation and reprogramming of the tumor microenvironment, (iii) aberrant tumor vasculature and/or stroma, and (iv) hypoxia. In altered-excluded immune tumors, T-cells are found at the edge of tumor sites (invasive margin) without being able to infiltrate them. This ‘excluded’ phenotype reflects the intrinsic ability of the host immune system to effectively mount a T-cell-mediated immune response and the ability of the tumor to escape such response by physically hindering T-cell infiltration (FIG. 7A). See, e.g., in Galon, J., and Bruni, D., Approaches to treat immune hot, altered and cold tumors with combination immunotherapies, Nature Reviews Drug Discovery (18), March 2019, 197-218, incorporated herein in its entirety. An exemplified resected tumor having the characteristics of an altered-excluded immune tumor is illustrated in FIG. 7A.

The characteristics of a cold tumor are: (i) absence of T-cells within the tumor and at the tumor edges (low immunoscore), and (ii) failed T-cell priming (low tumor mutational burden, poor antigen presentation and intrinsic insensitivity to T-cell killing). Cold tumors can also show a low expression of PD-L1. Apart from being poorly infiltrated, cold tumors have also been described to be immunologically ignorant (scarcely expressing PD-L1) and characterized by high proliferation with low mutational burden (low expression of neoantigens) and low expression of antigen presentation machinery markers such as major histocompatibility complex class I (WIC I). See, e.g., in Galon, J., and Bruni, D., Approaches to treat immune hot, altered and cold tumors with combination immunotherapies, Nature Reviews Drug Discovery (18), March 2019, 197-218, incorporated herein in its entirety. An exemplified resected tumor having the characteristics of cold immune tumor is illustrated in FIG. 7A.

Nonimmunogenic, or “cold” tumors have not yet been infiltrated with T cells, a sign that the immune response is not working in these tumors. The lack of T cells makes it difficult to provoke an immune response with immunotherapy drugs. The microenvironment surrounding cold tumors contains myeloid-derived suppressor cells (MDSC) and T regulatory cells (Tregs), which are known to dampen the immune response and inhibit T cells trying to move into the tumor. Additional features of cold tumors include lack of tumor antigens, defect in antigen presentation, absence of T cell activation and deficit of CD8+ homing into the tumor bed.

These types of tumors are mostly treated with traditional cancer therapies since checkpoint inhibitors and immunotherapy approaches have not been effective. Some breast cancers, ovarian cancer, prostate cancer, pancreatic cancer, neuroblastoma, small-cell lung cancer, and glioblastomas are typically cold tumors.

The determination of the immunogenic classification of a tumor can be carried out on resected tumors (primary or metastatic) (see e.g., FIG. 7A). Less invasive diagnostic procedures, such as immuno-positron-emission tomography (PET) imaging detecting intratumoral CD8+ T-cells can also be used. For a description of immuno-PET detection of intratumoral CD8+ T-cells, see Rooney et al., Molecular and genetic properties of tumors associated with local immune cytolytic activity, Cell 160, 48-61 (2015), incorporated herein by reference.

In addition to the above, bulk gene expression profiling can be used to determine the immunogenic classification of a tumor (see Ali et al., Patterns of immune infiltration in breast cancer and their clinical implications: a gene-expression-based retrospective study, PLOS Med. 13, e1002194 (2016); Newman et al., Robust enumeration of cell subsets from tissue expression profiles, Nat. Methods 12, 453-457 (2015); Rooney et al., Molecular and genetic properties of tumors associated with local immune cytolytic activity, Cell 160, 48-61 (2015), Bindea, G. et al., Spatiotemporal dynamics of intratumoral immune cells reveal the immune landscape in human cancer. Immunity 39, 782-795 (2013), each of which is incorporated herein by reference).

Multiple additional tools, such as CIBERSORT (which infers the relative fractions of immune subsets in the total leukocyte population), xCell (which predicts the abundance of immune cells in the overall TME), TIMER (which generates enrichment scores on the basis of proportions among 64 immune and stromal cell types) and integrated immunogenomics methods (using a CIBERSORT-based approach, which, of note, identified six immune subtypes of cancer) can be used to estimate the abundance of intra-tumoral immune infiltrates by using deconvolution of bulk gene expression data (see Newman et al., Robust enumeration of cell subsets from tissue expression profiles, Nat. Methods 12, 453-457 (2015); Gentles et al., The prognostic landscape of genes and infiltrating immune cells across human cancers, Nat. Med. 21, 938-945 (2015); Aran et al., xCell: digitally portraying the tissue cellular heterogeneity landscape, Genome Biol. 18, 220 (2017); Li et al., TIMER: a web server for comprehensive analysis of tumor-infiltrating immune cells, Cancer Res. 77, e108-e110 (2017); Thorsson, V. et al., The immune landscape of cancer. Immunity 48, 812-830 (2018), each incorporated herein by reference).

Immunoscore

Immunoscore is a digital pathology, IHC-based immune assay measuring the densities of CD3+ and CD8+ T cells at different tumor locations. The Immunoscore scoring has been defined in a large international SITC-led retrospective validation study conducted on more than 2500 St I-III colon cancer patients (see Pages et al, International validation of the consensus Immunoscore for the classification of colon cancer: a prognostic and accuracy study, The Lancet Volume 391, ISSUE 10135, P2128-2139, May 26, 2018, incorporated herein by reference). Commercial Immunoscore assays are available through, for example, HalioDx, Inc. (Richmond, Va.). Briefly, CD3- and CD8-immunostained formalin-fixed, paraffin-embedded (FFPE) slides are scanned and the two corresponding digital images validated by the operator. Image analysis is performed via a dedicated software (Immunoscore Analyzer, HalioDx): automatic detection of the tissue histologic structure is followed by an operator-guided definition of the tumor, healthy tissue (submucosa, muscularis propria, serosa), and the epithelium (mucosa). The operator also excludes all areas of necrosis, abscess, and artifacts (bubbles folds, torn areas, background) to avoid false positives. The IM, spanning 360 μm into the healthy tissue and 360 μm into the tumor, is calculated automatically by the software. In the presence of multiple FFPE blocks, the one to select for the Immunoscore evaluation is the one containing the IM.

Ayers Immune Scores

An additional measure for predicting the anti-tumor effect of CDK4/6 inhibitor therapy is determining the tumor's IFN-γ Signature Score and/or Expanded Immune Signature Score as described in Ayers et al. Ayers M, et al. “IFN-γ-Related MRNA Profile Predicts Clinical Response to PD-1 Blockade.” Journal of Clinical Investigation, vol. 127, no. 8, 2017, pp. 2930-2940, doi:10.1172/jci91190 (incorporated herein by reference in its entirety), who outline a thorough, iterative approach to building a gene expression signature predictive of response to immune checkpoint inhibitors (e.g., pembrolizumab). Starting in melanoma data, one-sided t-tests were used to detect differentially expressed genes between responders and non-responders to pembrolizumab. Noting that many of these differentially expressed genes were associated with IFN-γ signaling, Ayers et al. developed a preliminary IFN-γ signature for ICI response by averaging expression within the IFN-γ pathway and its correlated genes.

The robustness of the preliminary signatures of response was assessed in additional melanomas, HNSCCs and gastric cancers. Preliminary signatures displayed significant association with BOR and PFS, but not OS. To improve prediction in non-melanoma cancers, the immune signatures were trimmed and expanded by assessing univariate associations between individual genes and BOR and PFS within the expanded cohort. Genes with statistically insignificant associations were pruned from the signature and genes with significant associations were added to form intermediate IFN-gamma signatures.

Citing the success of the preliminary and intermediate IFN-gamma signatures in distinguishing responders and non-responders to anti-PD1 therapies as proof-of-concept for the extension of such predictive signatures to multiple cancer types, a final signature for multiple cancer types was developed using a wide array of samples from KEYNOTE-012 (ClinicalTrials.gov identifier: NCT01848834) and KENYNOTE-028 trials (ClinicalTrials.gov identifier: NCT02054806). Penalized logistic regression on BOR was used to further limit the intermediate signatures to a final set of 18 PD-1/PD-L1-response related genes.

The IFN-γ Signature analysis consists of determining the expression profile of six genes: IDO1, CXCL10, CXCL9, HLA-DRA; STAT1, and IFN-γ. The Expanded Immune Signature analysis consists of determining the expression profile of 18 genes: CCL5, CD27, CD274, CD276, CD8A, CMKLR1, CXCL9, CXCR6, HLA-DRB1, HLA-DQA1, HLA-E, IDO1, LAG3, NKG7, PDCD1LG2, PSMB10, STAT1, and TIGIT.

Ayers et al. performed sequencing quantitation using a 680 gene panel on the Nanostring platform. To compute a sample's score for either multi-gene signature (IFN-γ Signature or Expanded Immune Signature), quantile normalization is performed prior to a log 10 transformation and subsequent averaging across the gene-set. Calculation of the area under the ROC curve was used as a measure of discriminatory ability for the signature scores. The Youden index, a summary measure of the ROC curve (see Youden W J. Index for rating diagnostic tests. Cancer. 1950; 3(1):32-35, incorporated herein by reference), was used as an agnostic method for choosing an “optimal” cutoff, that is “high”/“low” on the signature scores to illustrate potential clinical usefulness. For example, a “high” IFN-γ Signature or Expanded Immune Signature can be determined based on comparison to scores of known immunogenic samples. In some embodiments, a “high” IFN-γ Signature or Expanded Immune Signature score is one that is scored greater than at least 2.25, 2.5, or 2.75. In some embodiments, a “high” IFN-γ Signature or Expanded Immune Signature score is one that is scored greater than at least 2.5.

The assessment provides a tumor type-independent applicability of a T-cell-inflamed gene expression profile that captures the biology of a T-cell inflamed microenvironment and as shown in the example below, TNBC patients having high IFN-γ Signature Scores and/or Enhanced Immune Signature Scores who are administered a CDK4/6 inhibitor show statistically significant overall survival improvements compared to those who do not receive a CDK4/6 inhibitor.

Thorsson et al. Six Class Immune Signature

An additional measure for predicting the anti-tumor effect of CDK4/6 inhibitor therapy is determining the tumor's Six Class Immune Signature as described by Thorsson et al., “The Immune Landscape of Cancer.” Immunity, vol 51, no. 2, 2018, pp. 812-830 (incorporated herein by reference in its entirety). Thorsson et al. performed an extensive literature search for expression signatures which characterized various facets of the immune response. This search resulted in 160 signatures for examination. Weighted Gene Correlation Network Analysis (WGCNA) over the entire TCGA (The Cancer Genome Atlas) dataset was used to cluster these 160 signatures into 9 distinguishable signature-modules, or collections of signatures purported to measure consistent immune phenomena. Candidate signatures were then restricted to the signatures which most closely represented the “average profile” from each of the 9 modules.

Further investigation identified that 4 of these 9 representative signatures were not robust classifiers for the TCGA data and resulted in highly variable classifications dependent upon which samples were utilized for model training. This left 5 immune signatures for use in sample classifications, which are identified in Table 1 below.

TABLE 1
Thorsson et al. Signature Label

| Signature Label | Signature Label |
|---|---|
| CSF1_Response | Representative of activation of |
| | macrophages and monocytes |
| LIexpression_Score | Representative of overall |
| | lymphocyte infiltration |
| TGF-β | Representative of TGF-β response |
| Module3_IFN_Score | Representative of IFN-γ response |
| CHANG_CORE_SERUM_RE- | Representative of wound healing |
| SPONSE_UP | (i.e. angiogenesis, etc) |

Scores for each of these five signatures were computed across the TCGA dataset using single sample Gene Set Enrichment Analysis (ssGSEA). These data were then clustered using an unsupervised, consensus clustering approach resulting in six identified immune response subtypes, as described in Table 2.

TABLE 2
Thorsson et al. Six Class Immune Signature

| Group | Description |
|---|---|
| C1 | Wound Healing: High proliferation rate, high angiogenesis |
| | gene expression, TH2 bias |
| C2 | IFN-γ Dominant: High M1/M2 polarization, Strong CD8 |
| | and high TCR diversity |
| C3 | Inflammatory: |
| | Elevated Th17 and Th1 genes, low to moderate proliferation, |
| | low aneuploidy and overall somatic copy number alterations. |
| C4 | Lymphocyte Depleted: |
| | Prominent macrophage signature with Th1 suppression and |
| | high M2 response |
| C5 | Immunologically Quiet: |
| | Low lymphocyte response, High macrophage response domi- |
| | nated by M2. |
| C6 | TGF-β Dominant: |
| | Mixed tumour subgroup with high TGF-β and lymphocytic |
| | infiltration |

As described in Example 3, TNBC patients with tumors in the C2 “IFN-γ Dominant” category administered a CDK4/6 inhibitor show statistically significant overall survival improvements compared to those that are C2 “IFN-γ Dominant” that do not receive a CDK4/6 inhibitor during treatment.

PD-L1 Status

An additional measure for predicting the anti-tumor effect of CDK4/6 inhibitor therapy is determining the programmed death-1 ligand (PD-L1) status of the tumor. PD-L1 is a transmembrane protein that down-regulates immune responses through binding to its two inhibitory receptors, programmed death-1 (PD-1) and B7.1. PD-1 is an inhibitory receptor expressed on T cells following T-cell activation, which is sustained in states of chronic stimulation such as in chronic infection or cancer (Blank, C and Mackensen, A, Contribution of the PD-L1/PD-1 pathway to T-cell exhaustion: an update on implications for chronic infections and tumor evasion. Cancer Immunol Immunother, 2007. 56(5): p. 739-745). Binding of PD-L1 with PD-1 inhibits T cell proliferation, cytokine production and cytolytic activity, leading to the functional inactivation or exhaustion of T cells. B7.1 is a molecule expressed on antigen presenting cells and activated T cells. PD-L1 binding to B7.1 on T cells and antigen presenting cells can mediate down-regulation of immune responses, including inhibition of T-cell activation and cytokine production (see Butte M J, Keir M E, Phamduy T B, et al. Programmed death-1 ligand 1 interacts specifically with the B7-1 costimulatory molecule to inhibit T cell responses. Immunity. 2007; 27(1):111-122). PD-L1 expression has been observed in immune cells and tumor cells. See Dong H, Zhu G, Tamada K, Chen L. B7-H1, a third member of the B7 family, co-stimulates T-cell proliferation and interleukin-10 secretion. Nat Med. 1999; 5(12):1365-1369; Herbst R S, Soria J C, Kowanetz M, et al. Predictive correlates of response to the anti-PD-L1 antibody MPDL3280A in cancer patients. Nature. 2014; 515(7528):563-567. Aberrant expression of PD-L1 on tumor cells has been reported to impede anti-tumor immunity, resulting in immune evasion. Therefore, interruption of the PD-L1/PD-1 pathway represents an attractive strategy to reinvigorate tumor-specific T cell immunity suppressed by the expression of PD-L1 in the tumor microenvironment. In cancer, the upregulation of PD-L1 may allow cancers to evade the host immune system.

PD-L1 expression can be determined by methods known in the art. For example, PD-L1 expression can be detected using PD-L1 IHC 22C3 pharmDx, the FDA-approved in vitro diagnostic immunohistochemistry (IHC) test developed by Dako and Bristol-Meyers Squibb as a companion test for treatment with pembrolizumab. This is qualitative assay using Monoclonal Mouse Anti-PD-L1, Clone 22C3 PD-L1 and EnVision FLEX visualization system on Autostainer Lin 48 to detect PD-L1 in formalin-fixed, paraffin-embedded (FFPE) human non-small cell lung cancer tissue. Expression levels can be measured using the tumor proportion score (TPS), which measures the percentage of viable tumor cells showing partial or complete membrane staining. Staining can show PD-L1 expression from 1% to 100%.

PD-L1 expression can also be detected using PD-L1 IHC 28-8 pharmDx, the FDA-approved in vitro diagnostic immunohistochemistry (IHC) test developed by Dako and Merck as a companion test for treatment with nivolumab. This qualitative assay uses the Monoclonal rabbit anti-PD-L1, Clone 28-8 and EnVision FLEX visualization system on Autostainer Lin 48 to detect PD-L1 in formalin-fixed, paraffin-embedded (FFPE) human non-small cell lung cancer tissue.

Other commercially available tests for PD-L1 detection include the Ventana SP263 assay (developed by Ventana in collaboration with AstraZeneca) that utilizes monoclonal rabbit anti-PD-L1, Clone SP263 and the Ventana SP142 Assay (developed by Ventana in collaboration with Genentech/Roche) that uses rabbit monoclonal anti-PD-L1 clone SP142. Determination of PD-L1 status is indication-specific, and evaluation is based on either the proportion of tumor area occupied by PD-L1 expressing tumor-infiltrating immune cells (% IC) of any intensity or the percentage of PD-L1 expressing tumor cells (% TC) of any intensity. For example, PD-L1 expression in ≥5% IC determined by, for example, the Ventana PD-L1 (SP142) Assay in urothelial carcinoma tissue, whereas a PD-L1 positive status in TNBC is considered ≥1% IC and NSCLC is considered ≥50% TC or ≥10% IC.

Short-Acting CDK4/6 Inhibitors

Short-acting CDK4/6 inhibitors for use in the present invention include Compound I, Compound II, Compound III, Compound IV, and Compound V, or pharmaceutically acceptable salts thereof.

Compound I, known as trilaciclib (2′-((5-(4-methylpiperazin-1-yl)pyridin-2-yl)amino)-7′, 8′-dihydro-6′H-spiro(cyclohexane-1,9′-pyrazino(1′,2′:1,5)pyrrolo(2,3-d)pyrimidin)-6′-one) is a highly selective CDK4/6 inhibitor having the structure:

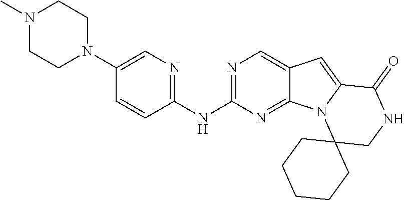 [SMILES: CN1CCN(c2ccc(Nc3ncc4cc5n(c4n3)C3(CCCCC3)CNC5=O)nc2)CC1]

As provided herein, trilaciclib or its pharmaceutically acceptable salt, composition, isotopic analog, or prodrug thereof is administered in a suitable carrier in a chemotherapeutic regime that includes an immune-response inducing chemotherapy such as an ICD-inducing chemotherapeutic. Trilaciclib is described in U.S. Pat. No. 8,598,186, incorporated herein by reference in its entirety. Trilaciclib can be synthesized as described in WO 2019/0135820, incorporated herein by reference in its entirety.

Trilaciclib, in one embodiment, may be administered parenterally, for example, intravenously, to a patient prior to administration of an immune-response inducing chemotherapy such as an ICD-inducing chemotherapy. In some embodiments, trilaciclib is administered up to about 24 hours or less, or up to about 20, 15, 10, 5, or 4 hours or less for example about 30-60 minutes or less, prior to administration of the chemotherapy. In some embodiments, trilaciclib is administered approximately about 22 to 26 hours before administration of the chemotherapy, and again about 4 hours or less, for example about 30-60 minutes or less, prior to administration of the chemotherapy. In some embodiments, the dose of trilaciclib administered is between about 180 and about 280 mg/m². For example, the dose is up to about 100, 125, 150, 180, 185, 190, 195, 200, 205, 210, 215, 220, 225, 230, 235, 240, 245, 250, 255, 260, 265, 270, 275, or 280 mg/m² or any dose in between these numbers as determined desirable by the healthcare practitioner. In a particular embodiment, the dose is about 240 mg/m².

Trilaciclib can be administered in any manner that achieves the desired outcome, including systemically, parenterally, intravenously, intramuscularly, subcutaneously, or intradermally. For injection, trilaciclib may be provided, in one embodiment, for example, as a 300 mg/vial as a sterile, lyophilized, yellow cake providing 300 mg of trilaciclib (equivalent to 349 mg of trilaciclib dihydrochloride). The product, for example, may be supplied in single-use 20-mL clear glass vials and does not contain a preservative. Prior to administration, trilaciclib for injection, 300 mg/vial may be reconstituted with 19.5 ml of 0.9% sodium chloride injection or 5% dextrose injection. This reconstituted solution has a trilaciclib concentration of 15 mg/mL and would typically be subsequently diluted prior to intravenous administration.

In an alternative embodiment, the Compound III, known as lerociclib, or its pharmaceutically acceptable salt, is administered instead of trilaciclib. Lerociclib (2′-((5-(4-isopropylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one) has the chemical structure:

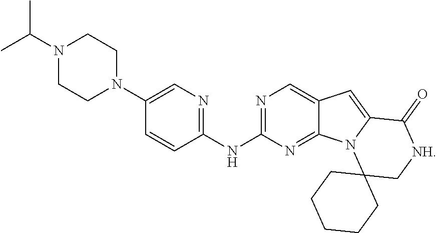 [SMILES: CC(C)N1CCN(c2ccc(Nc3ncc4cc5n(c4n3)C3(CCCCC3)CNC5=O)nc2)CC1]

Lerociclib can be administered in any manner that achieves the desired effect, including systemically, parenterally, orally, intravenously, intramuscularly, subcutaneously, or intradermally. Lerociclib can be prepared as previously described in WO 2014/144325, incorporated herein by reference. In some embodiments, lerociclib is administered using the same suggested amounts and methods as above for trilaciclib.

In a further alternative embodiment, the CDK4/6 inhibitor having the structure:

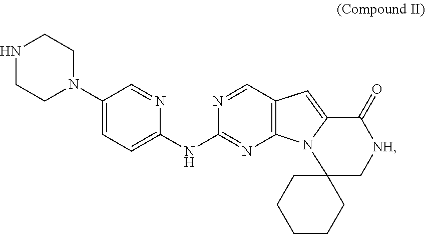 [SMILES: O=C1NCC2(CCCCC2)n2c1cc1cnc(Nc3ccc(N4CCNCC4)cn3)nc12]

or its pharmaceutically acceptable salt, is administered instead of trilaciclib. In some embodiments, this compound is administered using the same suggested amounts and methods as above for trilaciclib.

In a further alternative embodiment, the CDK4/6 inhibitor having the structure:

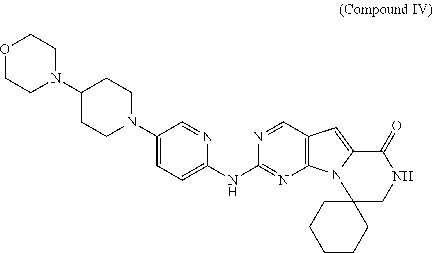 [SMILES: O=C1NCC2(CCCCC2)n2c1cc1cnc(Nc3ccc(N4CCC(N5CCOCC5)CC4)cn3)nc12]

or its pharmaceutically acceptable salt, is administered instead of trilaciclib. In some embodiments, this compound is administered using the same suggested amounts and methods as above for trilaciclib.

In a further alternative embodiment, the CDK4/6 inhibitor having the structure:

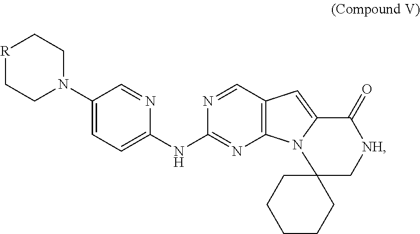

wherein R is C(H)X, NX, C(H)Y, or C(X)₂,

where X is straight, branched or cyclic C₁ to C₅ alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and

Y is NR₁R₂ wherein R₁ and R₂ are independently X, or wherein R₁ and R₂ are alkyl groups that together form a bridge that includes one or two heteroatoms (N, O, or S); and wherein two X groups can together form an alkyl bridge or a bridge that includes one or two heteroatoms (N, S, or O) to form a spiro compound, or its pharmaceutically acceptable salt, is administered instead of trilaciclib. In some embodiments, a compound selected from this formula is administered using the same suggested amounts and methods as above for trilaciclib.

In an alternative embodiment, a CDK4/6 inhibitor other than those specifically described above can be used in the present invention. Non-limiting examples include palbociclib, abemaciclib, and ribociclib.

Alternatively, the CDK4/6 inhibitor may be formulated as any pharmaceutically useful form, e.g., as a pill, an injection or infusion solution, a capsule, a tablet, a syrup, a transdermal patch, a subcutaneous patch, a subcutaneous injection, a dry powder, buccal, or sublingual formulation, parenteral formulation, or other suitable administration formulation.

Chemotherapies Capable of Inducing an Immune-Mediated Response

Standard cancer chemotherapy can promote tumor immunity in two major ways: (i) inducing immunogenic cell death as part of its intended therapeutic effect; and (ii) disrupting strategies that tumors use to evade the immune response. A large body of data demonstrates that some chemotherapy drugs at their standard dose and schedule mediate their antitumor effect, at least in part, by inducing immunogenic cell death (see, e.g., Emens et al., Chemotherapy: friend of foe to cancer vaccines? Curr Opin Mol Ther 2001; 3:77-84; Vanmeerbeek et al., Trial Watch: Chemotherapy-Induced Immunogenic Cell Death in Immuni-Oncology. Oncoimmunology Vol. 9, No. 1 2020:e1703449, both incorporated by reference herein).

Immunogenic cell death (ICD) is a type of cell death characterized by, for example, cell surface translocation of calreticulin (CRT), extracellular release of ATP and high mobility group box 1 (HMBG1), and stimulation of type I interferon (IFN) responses. ICD in cancer cells may prime an anticancer immune response. A variety of chemotherapeutic agents can induce ICD, as indicated by the alterations in tumor-infiltrating lymphocytes (TIL) abundance and composition.

In response to ICD-inducing chemotherapeutics, tumor cells expose CRT on cell surface prior to death, and release damage-associated molecular pattern (DAMP) molecules such as ATP during apoptosis or HMGB1 upon secondary necrosis. These DAMPs stimulate the recruitment of dendritic cells (DCs) into the tumor bed, the uptake and processing of tumor antigens, and the optimal antigen presentation to T cells. Cross-priming of CD8+ T-cells is triggered by mature DCs and γδ T-cells in an IL-1β and IL-17 dependent manner. Primed CTLs then elicit a direct cytotoxic response to kill remaining tumor cells through the generation of IFN-γ, perforin-1 and granzyme B.

ICD-inducing chemotherapies for use in the present invention include alkylating agents such as cyclophosphamide, trabectedin, temozolomide, melphalan, dacarbazine, and oxaliplatin; antimetabolites such as methotrexate, mitoxantrone, gemcitabine, and 5-fluorouracil (5-FU); cytotoxic antibiotics such as bleomycin and anthracyclines, including doxorubicin, daunorubicin, epirubicin, idarubicin, and valrubicin; taxanes, such as paclitaxel, cabazitaxel, and docetaxel; topoisomerase inhibitors such as topotecan, irinotecan, and etoposide; platinum compounds such as carboplatin and cisplatin; anti-microtubule Vinca alkaloid agents such as vinblastine, vincristine, vinorelbine, and vindesine. Other ICD-inducing chemotherapies include bortezomib, an inhibitor of the 26S proteasome subunit, mechlorethamine, diaziquone, mitomycin C, fludarabine and cytosine arabinoside. In some embodiments, the ICD-inducing chemotherapy is selected from idarubicin, epirubicin, doxorubicin, mitoxantrone, oxaliplatin, bortezomib, gemcitabine, and cyclophosphamide, and combinations thereof. In an alternative embodiment, the chemotherapeutic administered is capable of inducing an immune-response may modulate tumor immunity by mechanisms distinct from immunogenic cell death. Various chemotherapy drugs can modulate the activity of distinct immune cell subsets or the immune phenotype of tumor cells through enhancing antigen presentation, enhancing expression of costimulatory molecules including B7.1 (CD80) and B7.2 (CD86), downregulating checkpoint molecules such as programmed death-ligand 1 (PD-L1), or promoting tumor cell death through the fas, perforin, or Granzyme B pathways. Chemotherapies that modulate tumor immunity may do so by: abrogating myeloid-derived suppressor cell (MDSC) activity, for example gemcitabine, 5-fluoruracil, cisplatin, and doxorubicin; abrogating Treg activity, for example cyclophosphamide, 5-fluorouracil; paclitaxel, cisplatin, and fludarabine; enhancement of T-cell cross priming, for example gemcitabine and anthracyclines such as doxorubicin, daunorubicin, epirubicin, valrubicin and idarubicin; augmenting dendritic cell activation, for example anthracyclines, taxanes, cyclophosphamide, Vinca alkaloids, methotrexate, and mitomycin C; promoting anti-tumor CD4+ T-cell phenotype, for example cyclophosphamide and paclitaxel; and promoting tumor cell recognition and lysis, for example cyclophosphamide, 5-fluorouracil, paclitaxel, doxorubicin, cisplatin, and cytosine arabinoside.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of an alkylating agent such as cyclophosphamide, trabectedin, temozolomide, melphalan, dacarbazine, or oxaliplatin; an antimetabolite such as methotrexate, mitoxantrone, gemcitabine, or 5-fluorouracil (5-FU); a cytotoxic antibiotic such as bleomycin or an anthracycline such as doxorubicin, daunorubicin, epirubicin, idarubicin, or valrubicin; a taxane, such as paclitaxel, cabazitaxel, and docetaxel; topoisomerase inhibitors such as topotecan, irinotecan, and etoposide; platinum compounds such as carboplatin and cisplatin; anti-microtubule Vinca alkaloid agents such as vinblastine, vincristine, vinorelbine and vindesine; bortezomib; mechlorethamine; diaziquone; fludarabine; mitomycin C; and cytosine arabinoside. In some embodiments, the administration of the CDK4/6 inhibitor in combination with the chemotherapy does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of cyclophosphamide. In some embodiments, the administration of the short acting CDK4/6 inhibitor in combination with cyclophosphamide does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of trabectedin. In some embodiments, the administration of the short acting CDK4/6 inhibitor in combination with trabectedin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of temozolomide. In some embodiments, the administration of the short acting CDK4/6 inhibitor in combination with temozolomide does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of melphalan. In some embodiments, the administration of the short acting CDK4/6 inhibitor in combination with melphalan does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of dacarbazine. In some embodiments, the administration of the short acting CDK4/6 inhibitor in combination with dacarbazine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of oxaliplatin. In some embodiments, the administration of the short acting CDK4/6 inhibitor in combination with oxaliplatin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of methotrexate. In some embodiments, the administration of the short acting CDK4/6 inhibitor in combination with methotrexate does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of 5-fluorouracil (5-FU). In some embodiments, the administration of the short acting CDK4/6 inhibitor in combination with 5-FU does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of gemcitabine. In some embodiments, the administration of the short acting CDK4/6 inhibitor in combination with gemcitabine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of mitoxantrone. In some embodiments, the administration of the short acting CDK4/6 inhibitor in combination with mitoxantrone does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of doxorubicin. In some embodiments, the administration of the short acting CDK4/6 inhibitor in combination with doxorubicin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of daunorubicin. In some embodiments, the administration of the short acting CDK4/6 inhibitor in combination with daunorubicin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of idarubicin. In some embodiments, the administration of the short acting CDK4/6 inhibitor in combination with idarubicin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of valrubicin. In some embodiments, the administration of the short acting CDK4/6 inhibitor in combination with valrubicin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of epirubicin. In some embodiments, the administration of the short acting CDK4/6 inhibitor in combination with epirubicin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of bleomycin. In some embodiments, the administration of the short acting CDK4/6 inhibitor in combination with bleomycin does not include administering an immune checkpoint inhibitor. In some embodiments, the patient has a hot immune tumor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of bortezomib. In some embodiments, the administration of the short acting CDK4/6 inhibitor in combination with bortezomib does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of paclitaxel. In some embodiments, the administration of the short acting CDK4/6 inhibitor in combination with paclitaxel does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of docetaxel. In some embodiments, the administration of the short acting CDK4/6 inhibitor in combination with docetaxel does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of cabazitaxel. In some embodiments, the administration of the short acting CDK4/6 inhibitor in combination with cabazitaxel does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of topotecan. In some embodiments, the administration of the short acting CDK4/6 inhibitor in combination with topotecan does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of etoposide. In some embodiments, the administration of the short acting CDK4/6 inhibitor in combination with etoposide does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of irinotecan. In some embodiments, the administration of the short acting CDK4/6 inhibitor in combination with irinotecan does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of cisplatin. In some embodiments, the administration of the short acting CDK4/6 inhibitor in combination with cisplatin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of carboplatin. In some embodiments, the administration of the short acting CDK4/6 inhibitor in combination with carboplatin does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of vinblastine. In some embodiments, the administration of the short acting CDK4/6 inhibitor in combination with vinblastine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of vincristine. In some embodiments, the administration of the short acting CDK4/6 inhibitor in combination with vincristine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of vinorelbine. In some embodiments, the administration of the short acting CDK4/6 inhibitor in combination with vinorelbine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of vindesine. In some embodiments, the administration of the short acting CDK4/6 inhibitor in combination with vindesine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of diaziquone. In some embodiments, the administration of the short acting CDK4/6 inhibitor in combination with diaziquone does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of mechlorethamine. In some embodiments, the administration of the short acting CDK4/6 inhibitor in combination with mechlorethamine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of mitomycin C. In some embodiments, the administration of the short acting CDK4/6 inhibitor in combination with mitomycin C does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of fludarabine. In some embodiments, the administration of the short acting CDK4/6 inhibitor in combination with fludarabine does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In some embodiments, a method for selecting a patient or patient population for cancer therapy that includes the administration of a CDK 4/6 inhibitor with chemotherapy in a manner that increases the progression free survival or overall survival of the patient or patient population is provided comprising, determining if the cancer has a surrounding microenvironment that is favorable to immune modulation, is immunogenically susceptible to CDK4/6 inhibitor treatment, or is immunogenic, and if so, administering to the patient an effective amount of a CDK4/6 inhibitor in combination with an effective amount of cytosine arabinoside. In some embodiments, the administration of the short acting CDK4/6 inhibitor in combination with cytosine arabinoside does not include administering an immune checkpoint inhibitor. In some embodiments, the patient has a tumor classified as immunogenic. In some embodiments, the patient has a hot immune tumor. In some embodiments, the patient has an altered-immunosuppressed immune tumor. In some embodiments, the patient has an altered-excluded immune tumor. In some embodiments, the patient has a cold tumor. In some embodiments, the patient has a tumor that is classified as a C2 “IFN-γ Dominant” class cancer. In some embodiments, the patient has a tumor that is classified as a high “IFN-γ Signature” or a high “Expanded Immune Signature.” In some embodiments, the patient has a tumor that is PD-L1 positive. In some embodiments, the CDK4/6 inhibitor administered is Compound I, or a pharmaceutically acceptable salt therein. In some embodiments, the CDK4/6 inhibitor administered is Compound III, or a pharmaceutically acceptable salt therein.

In any of the above embodiments, the patient to be treated has been determined to have a cancer having a surrounding microenvironment that is favorable to immune modulation, is immunogenic, or is immunogenically susceptible to CDK4/6 inhibitor treatment. Accordingly, provided the cancer fits into the category as described herein, the patient may be suitable for the described treatments. In some embodiments, the cancer to be treated is selected from the group consisting of breast cancer, including but not limited to estrogen receptor (ER)-positive breast cancer and triple negative breast cancer, non-small cell lung carcinoma, head and neck squamous cell cancer, classical Hodgkin lymphoma (cHL), diffuse large B-cell lymphoma, bladder cancer, primary mediastinal B-cell lymphoma (PBMCL), urothelial carcinoma, microsatellite instability-high (MSI—H) solid tumors, mismatch repair deficient (dMMR) solid tumor, gastric or gastroesophageal junction (GEJ) adenocarcinoma, squamous cell carcinoma of the esophagus, cervical cancer, endometrial cancer, cholangiocarcinoma, hepatocellular carcinoma, Merkel cell carcinoma, renal cell carcinoma, ovarian cancer, anal canal cancer, colorectal cancer, skin cutaneous melanoma and melanoma.

In some embodiments, provided herein is a method for selecting a patient or patient population for triple negative breast cancer therapy that includes the administration of a CDK 4/6 inhibitor with chemotherapy in a manner that increases the progression free survival or overall survival of the patient or patient population comprising administering to the patient gemcitabine and carboplatin and administering a CDK 4/6 inhibitor selected from Compound I, Compound II, Compound III, Compound IV, or a pharmaceutically acceptable salt thereof, wherein the CDK4/6 inhibitor is administered prior to the administration of gemcitabine and carboplatin, and wherein the cancer is, prior to initiation of treatment, determined to be immunogenic, immunogenically susceptible to CDK4/6 inhibitor treatment, or have a surrounding microenvironment that is favorable to immune modulation.

In some embodiments, for selecting a patient or patient population for triple negative breast cancer therapy that includes the administration of a CDK 4/6 inhibitor with chemotherapy in a manner that increases the progression free survival or overall survival of the patient or patient population comprising administering to the patient gemcitabine and carboplatin on Days 1 and 8 of 21-day cycles; administering a CDK 4/6 inhibitor selected from Compound I, Compound II, Compound III, Compound IV, or a pharmaceutically acceptable salt thereof, wherein the CDK4/6 inhibitor is administered prior to the administration of gemcitabine and carboplatin, and wherein the triple negative cancer is, prior to initiation of treatment, determined to be immunogenic, immunogenically susceptible to CDK4/6 inhibitor treatment, or have a surrounding microenvironment that is favorable to immune modulation.

Administration Protocols

The methods described herein provide for the administration of a CDK4/6 inhibitor with a chemotherapy capable of inducing an immune-mediated response in a cancer, for example an ICD-inducing chemotherapy for extending the overall survival or progression free survival of a patient with cancer, such methods including determining if the patient has a cancer that can be classified as immunogenic, or has a surrounding microenvironment that is favorable to immune modulation, or is susceptible to, or the cancer is immunogenically susceptible to CDK4/6 inhibitor treatment, and if so, administer to the patient a chemotherapeutic agent capable of inducing an immune-mediated response in combination with a CDK4/6 inhibitor.

In some embodiments, the CDK4/6 inhibitor is administered prior to or concomitantly with the administration of the chemotherapeutic agent. In some embodiments, the selective CDK4/6 inhibitor is administered to the subject less than about 24 hours, about 20 hours, about 16 hours, about 12 hours, about 8 hours, about 4 hours, about 2.5 hours, about 2 hours, about 1 hour, about ½ hour or less prior to treatment with the chemotherapeutic agent. In a particular embodiment, the selective CDK4/6 inhibitor is administered about ½ hour prior to administration of the chemotherapeutic agent.

Typically, the selective CDK4/6 inhibitor is administered to the subject prior to treatment with the chemotherapeutic agent such that the CDK4/6 inhibitor reaches peak serum levels before or during treatment with the chemotherapeutic agent, allowing for the inhibition of proliferation of immune effector cells, thus protecting them from the harmful effects of chemotherapy. In some embodiment, the CDK4/6 inhibitor is administered concomitantly, or closely thereto, with the chemotherapeutic agent exposure. In one embodiment, the selective CDK4/6 inhibitor is Compound I, or a pharmaceutically acceptable salt thereof. In one embodiment, the selective CDK4/6 inhibitor is Compound III, or a pharmaceutically acceptable salt thereof.

In some embodiments, the CDK4/6 inhibitor is administered to the subject less than about 24 hours, about 20 hours, about 16 hours, about 12 hours, about 8 hours, about 4 hours, about 2.5 hours, about 2 hours, about 1 hour, about ½ hour or less prior to treatment with the chemotherapeutic agent. In a particular embodiment, the selective CDK4/6 inhibitor is administered about ½ hour prior to administration of the chemotherapeutic agent. Typically, the CDK4/6 inhibitor is administered to the subject prior to treatment with the chemotherapeutic agent such that the CDK4/6 inhibitor reaches peak serum levels before or during treatment with the chemotherapeutic agent, allowing for the inhibition of proliferation of immune effector cells, thus protecting them from the harmful effects of chemotherapy. In one embodiment, the CDK4/6 inhibitor is administered concomitantly, or closely thereto, with the chemotherapeutic agent exposure. Alternatively, the CDK4/6 inhibitor described herein can be administered following exposure to the chemotherapeutic agent if desired to mitigate immune effector cell damage associated with chemotherapeutic agent exposure.

In some embodiments, the CDK4/6 inhibitor is administered to the subject twice before administration of the chemotherapy. For example, in some embodiments, the CDK4/6 inhibitor is administered between about 18 and 28 hours before the administration of the chemotherapy, and then once again at less than about 4 hours, about 2.5 hours, about 2 hours, about 1 hour, about ½ hour or less prior to treatment with the chemotherapeutic agent. In a particular embodiment, the selective CDK4/6 inhibitor is administered between about 22 and 26 hours prior to administration of the chemotherapeutic agent and again about ½ hour or less prior to administration of the chemotherapeutic agent.

In certain embodiments, the CDK4/6-inhibitor is administered prior to or concomitantly with the administration of a chemotherapeutic agent, wherein the chemotherapeutic agent is administered: for example, on day 1-3 every 21 days; on days 1-3 every 28 days; on day 1 every 3 weeks; on day 1, day 8, and day 15 every 28 days, on day 1 and day 8 every 28 days; on days 1 and day 8 every 21 days; on days 1-5 every 21 days; 1 day a week for 6-8 weeks; on days 1, 22, and 43; days 1 and 2 weekly; days 1-4 and 22-25; days 1-4; days 22-25, and days 43-46; and similar type chemotherapeutic regimens. In some embodiments, the CDK4/6 inhibitor is administered prior to or concomitantly with at least one administration of the chemotherapeutic agent during a chemotherapeutic treatment regimen. In some embodiments, the CDK4/6 is administered prior to or concomitantly with one or more administrations of the chemotherapeutic agent during a chemotherapeutic treatment regimen. In one embodiment, the CDK4/6 inhibitor is administered prior to or concomitantly with each administration of the chemotherapeutic agent during a chemotherapeutic treatment regimen.

In some embodiments, the CDK4/6 inhibitor is administered prior to or concomitantly with each administration of a chemotherapeutic agent for example during a standard chemotherapeutic protocol such as, for example, a 21-day cycle. Following cessation of the standard chemotherapeutic protocol, the CDK4/6 inhibitor is further administered alone in a maintenance dose. In some embodiments, the CDK4/6 inhibitor is further administered once a week for at least 3, 4, 5, 6, 7, 8, 9, 10, 11, 12, 13, 14, 15, 16, 26, 52, 104 weeks, or longer. In some embodiments, the CDK 4/6 inhibitor is administered once every 21 days following the cessation of the chemotherapeutic protocol. In one embodiment, the selective CDK4/6 inhibitor is a fast-acting, short half-life CDK4/6 inhibitor.

In some embodiments, the CDK4/6 inhibitor is administered with a chemotherapy agent in a maintenance therapy treatment regimen following cessation of the standard chemotherapeutic protocol. Maintenance therapy can comprise either continuation of an agent given as part of the first-line or previous regimen (continuation maintenance) or treatment with a new agent (switch maintenance).

In some embodiments, the CDK4/6 inhibitor is further administered in a maintenance-type therapeutic regimen, wherein the CDK4/6 inhibitor is administered in combination with a reduced maintenance dose of chemotherapy at a regular dosing interval for example but not limited to, once a week, once every two weeks, once every three weeks, once a month, once every six weeks, once every two months, once every three months, or once every six months following the completion of the initial chemotherapy treatment. In some embodiments, the CDK4/6 inhibitor is administered with the same agent used in the previous phase of chemotherapy treatment. In some embodiments, the CDK4/6 inhibitor is administered with a different chemotherapy agent than was used in the previous phase of chemotherapy treatment.

In some embodiments, the patient is not administered a check point inhibitor.

### EMBODIMENTS

The following embodiments are provided herein:
1. A method for selecting a patient or patient population for cancer therapy that includes the administration of a CDK 4/6 inhibitor with chemotherapy in a manner that increases the progression free survival or overall survival of the patient comprising:
   (i) determining if the patient's cancer has a surrounding microenvironment that is favorable to immune modulation;
   (ii) determining whether the chemotherapy regimen induces an immune-mediated response such as immunogenic cell death, and if both (i) and (ii) are yes, then,
   (iii) administering an effective amount of a CDK4/6 inhibitor selected from Compounds I, II, III, IV, or V, or a pharmaceutically acceptable salt thereof,

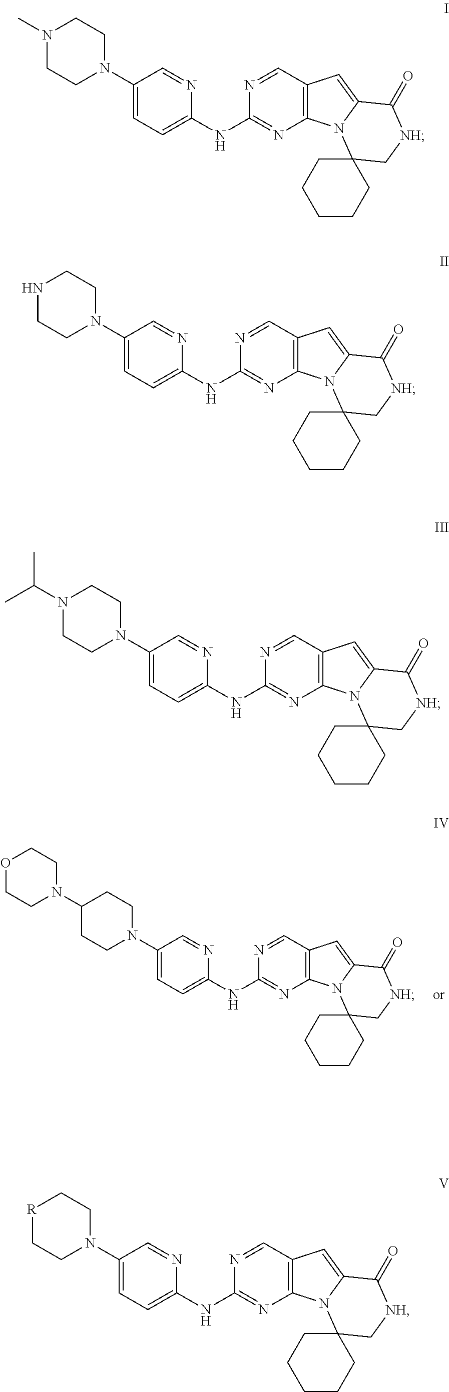

wherein R is C(H)X, NX, C(H)Y, or C(X)₂,
where X is straight, branched or cyclic C1 to C5 alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and
Y is NR₁R₂ wherein R₁ and R₂ are independently X, or wherein R₁ and R₂ are alkyl groups that together form a bridge that includes one or two heteroatoms (N, O, or S);
and wherein two X groups can together form an alkyl bridge or a bridge that includes one or two heteroatoms (N, S, or O) to form a spiro compound, or a pharmaceutically acceptable salt thereof;
wherein the CDK4/6 inhibitor is administered prior to the administration of the chemotherapy or optionally prior to and concurrently with chemotherapy; and, wherein the increase in progression free survival or overall survival is in comparison to the progression free survival or overall survival based on administration of the chemotherapy alone.
2. The method of embodiment 1, wherein the determination of whether the cancer has a surrounding microenvironment that is favorable to immune modulation comprises comparing a cancer tissue sample to those characterized in FIG. 7.
3. The method of embodiment 1, wherein the determination of whether the cancer has a surrounding microenvironment that is favorable to immune modulation comprises assessing the cancer according to FIG. 6.
4. The method of embodiment 1, wherein the determination of whether the cancer has a surrounding microenvironment that is favorable to immune modulation is according to the Galon immunoscore system.
5. The method of embodiment 1, wherein the determination of whether the cancer has a surrounding microenvironment that is favorable to immune modulation comprises assessing whether the cancer has a sufficiently high level of major histocompatibility complex class I antigens available to initiate an effective immune response.
6. The method of embodiment 1, wherein the determination of whether the cancer has a surrounding microenvironment that is favorable to immune modulation comprises assessing whether the cancer has a sufficiently high level of major histocompatibility complex class II antigens available to initiate an effective immune response.
7. The method of embodiment 1, wherein the determination of whether the cancer has a surrounding microenvironment that is favorable to immune modulation comprises assessing whether the cancer has a sufficiently high level of major histocompatibility complex class I and class II antigens available to initiate an effective immune response.
8. The method of any of embodiments 1-7, wherein the patient has a cancer that is immunogenically classified as a hot immune tumor.
9. The method of any of embodiments 1-7, wherein the patient has a cancer that is immunogenically classified as an altered-immunosuppressed tumor.
10. The method of embodiment 1-7, the determination of whether the cancer has a surrounding microenvironment that is favorable to immune modulation comprises assessing whether the cancer is an IFN-γ Dominant class cancer, has a cancer microenvironment with a high IFN-γ Signature, or has a high Expanded Immune Signature, PD-L1 positive, or combination thereof.
11. The method of any of embodiments 1-7, wherein the CDK4/6 inhibitor administered is Compound I, or a pharmaceutically acceptable salt thereof.
12. The method of any of embodiments 1-11, wherein the CDK4/6 inhibitor administered is Compound II, or a pharmaceutically acceptable salt thereof.
13. The method of any of embodiments 1-11, wherein the CDK4/6 inhibitor administered is Compound III, or a pharmaceutically acceptable salt thereof.
14. The method of any of embodiments 1-11, wherein the CDK4/6 inhibitor administered is Compound IV, or a pharmaceutically acceptable salt thereof
15. The method of any of embodiments 1-11, wherein the CDK4/6 inhibitor administered is Compound V, or a pharmaceutically acceptable salt thereof.
16. The method of any of embodiments 1-15, wherein the CDK4/6 inhibitor is administered about 24 hours or less prior to the administration of the chemotherapy.
17. The method of any of embodiments 1-15, wherein the CDK4/6 inhibitor is administered about 4 hours or less prior to the administration of the chemotherapy.
18. The method of any of embodiments 1-15, wherein the CDK4/6 inhibitor is administered about 30 minutes or less prior to the administration of the chemotherapy.
19. The method of any of embodiments 1-18, wherein the chemotherapy is chemotherapy is selected from the group consisting of cyclophosphamide, trabectedin, temozolomide, melphalan, dacarbazine, oxaliplatin, methotrexate, mitoxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations thereof
20. A method of selecting a patient or patient population for cancer therapy that includes the administration of a CDK 4/6 inhibitor with chemotherapy in a manner that increases the progression free survival or overall survival of the patient or patient population comprising:
   (i) determining whether the cancer is immunogenic;
   (ii) determining whether the patient can be administered an ICD-inducing chemotherapy based on the cancer;
   (iii) and, if it is determined that the cancer is immunogenic and an ICD-inducing chemotherapy can be administered, administering an effective amount of an ICD-inducing chemotherapy in combination with an effective amount of a short-acting CDK4/6 inhibitor selected from Compound I, Compound II, Compound III, Compound IV, or Compound V or pharmaceutically acceptable salt thereof, wherein the CDK4/6 inhibitor is administered prior to administration of the ICD-inducing chemotherapy or optionally prior to and concurrently with chemotherapy.
21. The method of embodiment 20, wherein the cancer immunogenic if the cancer has a surrounding microenvironment that is favorable to immune modulation comprises comparing a cancer tissue sample to those characterized in FIG. 7.
22. The method of embodiment 20, wherein the cancer is immunogenic if the cancer has a surrounding microenvironment that is favorable to immune modulation comprises assessing the cancer according to FIG. 6.
23. The method of embodiment 20, wherein the cancer is immunogenic if the cancer has a surrounding microenvironment that is favorable to immune modulation according to the Galon immunoscore system.
24. The method of embodiment 20, wherein the cancer is immunogenic if immunogenically classified as a hot immune tumor.
25. The method of embodiment 20, wherein the cancer is immunogenic if immunogenically classified as an altered-immunosuppressed immune tumor.
26. The method of embodiment 20, wherein the patient has a cancer that is immunogenically classified as an altered-excluded.
27. The method of embodiment 20, wherein the cancer is immunogenic if classified as an IFN-γ Dominant class cancer, has a cancer microenvironment with a high IFN-γ Signature, or has a high Expanded Immune Signature, PD-L1 positive, or combination thereof
28. The method of any of embodiments 20-27, wherein the CDK4/6 inhibitor administered is Compound I, or a pharmaceutically acceptable salt thereof
29. The method of any of embodiments 20-27, wherein the CDK4/6 inhibitor administered is Compound III, or a pharmaceutically acceptable salt thereof.
30. The method of any of embodiments 20-29, wherein the CDK4/6 inhibitor is administered about 24 hours or less prior to the administration of the ICD-inducing chemotherapy.
31. The method of any of embodiments 20-29, wherein the CDK4/6 inhibitor is administered about 4 hours or less prior to the administration of the ICD-inducing chemotherapy.
32. The method of any of embodiments 20-29, wherein the CDK4/6 inhibitor is administered about 30 minutes or less prior to the administration of the ICD-inducing chemotherapy.
33. The method of any of embodiments 20-29, wherein the CDK4/6 inhibitor is administered first about 22 to 26 hours prior to administration of the ICD-inducing chemotherapy, and again about 4 hours or less prior to administration of the ICD-inducing chemotherapy.
34. The method of any of embodiments 20-33, wherein the ICD-inducing chemotherapy is selected from the group consisting of cyclophosphamide, trabectedin, temozolomide, melphalan, dacarbazine, oxaliplatin, methotrexate, mitoxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations of thereof
35. The method of any of embodiments 1 to 34, wherein the patient is not administered an immune checkpoint inhibitor at the time of the administration of the CDK4/6 inhibitor.
36. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) selection of the patient or patient population based on the determination of whether the cancer has a surrounding microenvironment that is favorable to immune modulation; and determination if the chemotherapy regimen is capable of inducing an immune-mediated response, and
   (ii) wherein the CDK4/6 inhibitor is administered prior to the administration of the chemotherapy or optionally prior to and concurrently with chemotherapy;
wherein the increase in overall survival or progression free survival is in comparison to the overall survival or progression free survival based on administration of the chemotherapy alone.
37. The use of embodiment 36, wherein the determination of whether the cancer has a surrounding microenvironment that is favorable to immune modulation comprises comparing a cancer tissue sample to those characterized in FIG. 7.
38. The use of embodiment 36, wherein the determination of whether the cancer has a surrounding microenvironment that is favorable to immune modulation comprises assessing the cancer according to FIG. 6.
39. The use of embodiment 36, wherein the determination of whether the cancer has a surrounding microenvironment that is favorable to immune modulation is according to the Galon immunoscore system.
40. The use of embodiment 36, wherein the determination of whether the cancer has a surrounding microenvironment that is favorable to immune modulation comprises assessing whether the cancer microenvironment has a sufficiently high level of major histocompatibility complex class I antigens available to initiate an effective immune response.
41. The use of embodiment 36, wherein the determination of whether the cancer has a surrounding microenvironment that is favorable to immune modulation comprises assessing whether the cancer microenvironment has a sufficiently high level of major histocompatibility complex class II antigens available to initiate an effective immune response.
42. The use of embodiment 36, wherein the determination of whether the cancer has a surrounding microenvironment that is favorable to immune modulation comprises assessing whether the cancer microenvironment has a sufficiently high level of major histocompatibility complex class I and class II antigens available to initiate an effective immune response.
43. The use of any of embodiments 36, wherein the patient has a cancer that is immunogenically classified as a hot immune tumor.
44. The use of any of embodiments 36, wherein the patient has a cancer that is immunogenically classified as an altered-immunosuppressed tumor.
45. The use of embodiment 36, wherein the patient has a cancer that is a C2 IFN-γ Dominant class cancer, a cancer microenvironment with a high IFN-γ signature or a high expanded immune signature, or a cancer that is PD-L1 positive.
46. The use of embodiment 36, wherein the determination of whether the cancer has a surrounding microenvironment that is favorable to immune modulation comprises assessing whether the cancer microenvironment has a sufficiently high degree of T cell and cytotoxic T cell infiltration.
47. The use of any of embodiments 36-46, wherein the CDK4/6 inhibitor administered is Compound I, or a pharmaceutically acceptable salt thereof
48. The use of any of embodiments 36-46, wherein the CDK4/6 inhibitor administered is Compound III, or a pharmaceutically acceptable salt thereof.
49. The use of any of embodiments 36-46, wherein the CDK4/6 inhibitor is administered about 24 hours or less prior to the administration of the chemotherapy.
50. The use of any of embodiments 36-49, wherein the ICD-inducing chemotherapy is selected from the group consisting of cyclophosphamide, trabectedin, temozolomide, melphalan, dacarbazine, oxaliplatin, methotrexate, mitoxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations of thereof
51. The use of any of embodiments 36-49, wherein the chemotherapy is an immunogenic cell death (ICD)-inducing chemotherapy.
52. The use of any of embodiments 36-51, wherein the cancer is selected from the group consisting of triple negative breast cancer, non-small cell lung carcinoma, head and neck squamous cell cancer, classical Hodgkin lymphoma (cHL), bladder cancer, primary mediastinal B-cell lymphoma (PBMCL), urothelial carcinoma, microsatellite instability-high (MSI—H) solid tumors, mismatch repair deficient (dMMR) solid tumor, gastric or gastroesophageal junction (GEJ) adenocarcinoma, squamous cell carcinoma of the esophagus, cervical cancer, hepatocellular carcinoma, Merkel cell carcinoma, renal cell carcinoma, ovarian cancer, anal canal cancer, colorectal cancer, and melanoma.
53. The method of any of embodiments 36-52, wherein the patient is not administered an immune checkpoint inhibitor at the time of the administration of the CDK4/6 inhibitor.
54. Use of a compound selected from Compound I, Compound II, Compound III, Compound IV, Compound V, Compound V, or a pharmaceutically acceptable salt thereof, in the manufacture for cancer therapy to a selected patient or patient population in a manner that increases the progression free survival or overall survival of the patient or patient population comprising:
   (i) determining whether the cancer is immunogenically susceptible to CDK4/6 inhibitor treatment;
   (ii) determining whether the patient can be administered a chemotherapy capable of inducing an immune-mediated response, and
   (iii) if both (i) and (ii) are yes, administering an effective amount of the CDK 4/6 inhibitor, wherein the CDK4/6 inhibitor is administered prior to the administration of the chemotherapy or optionally prior to and concurrently with chemotherapy; and, wherein the improvement in overall survival or progression free survival is in comparison to the overall survival or progression free survival based on administration of the chemotherapy alone.
55. The use of embodiment 54, wherein the cancer is immunogenically susceptible to CDK4/6 inhibitor treatment if the cancer has a surrounding microenvironment that is favorable to immune modulation as assessed according to FIG. 6.
56. The use of embodiment 54, wherein the cancer is immunogenically susceptible to CDK4/6 inhibitor treatment if the cancer has a surrounding microenvironment that is favorable to immune modulation as assessed according to the Galon immunoscore system.
57. The use of embodiment 54, wherein the cancer is immunogenically susceptible to CDK4/6 inhibitor treatment if the cancer microenvironment has a sufficiently high level of major histocompatibility complex class I antigens available to initiate an effective immune response.
58. The use of embodiment 54, wherein the cancer is immunogenically susceptible to CDK4/6 inhibitor treatment if the cancer microenvironment has a sufficiently high level of major histocompatibility complex class II antigens available to initiate an effective immune response.
59. The use of embodiment 54, wherein the cancer is immunogenically susceptible to CDK4/6 inhibitor treatment if the cancer microenvironment has a sufficiently high level of major histocompatibility complex class I and class II antigens available to initiate an effective immune response.
60. The use of any of embodiments 54-59, wherein the patient has a cancer that is immunogenically classified as a hot immune tumor.
61. The use of any of embodiments 54-59, wherein the patient has a cancer that is immunogenically classified as an altered-immunosuppressed tumor.
62. The use of embodiment 54, wherein the patient has a cancer microenvironment that is a C2 IFN-γ Dominant class cancer, has a cancer microenvironment with a high IFN-γ Signature or a high Expanded Immune Signature, has a cancer that is PD-L1 positive.
63. The use of embodiment 54, wherein the cancer is immunogenically susceptible to CDK4/6 inhibitor treatment if the cancer microenvironment has a sufficiently high degree of T cell and cytotoxic T cell infiltration.
64. The use of any of embodiments 54-63, wherein the CDK4/6 inhibitor administered is Compound I, or a pharmaceutically acceptable salt thereof
65. The use of any of embodiments 54-63, wherein the CDK4/6 inhibitor administered is Compound III, or a pharmaceutically acceptable salt thereof.
66. The use of any of embodiments 54-65, wherein the CDK4/6 inhibitor is administered about 24 hours or less prior to the administration of the chemotherapy.
67. The use of any of embodiments 54-66, wherein the CDK4/6 inhibitor is administered about 4 hours or less prior to the administration of the chemotherapy.
68. The use of any of embodiments 54-66, wherein the CDK4/6 inhibitor is administered about 30 minutes or less prior to the administration of the chemotherapy.
69. The use of any of embodiments 54-69, wherein the ICD-inducing chemotherapy is selected from the group consisting of cyclophosphamide, trabectedin, temozolomide, melphalan, dacarbazine, oxaliplatin, methotrexate, mitroxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations of thereof.
70. The use of any of embodiments 54-69, wherein the chemotherapy is an immunogenic cell death (ICD)-inducing chemotherapy.
71. The use of any of embodiments 54-70, wherein the cancer is selected from the group consisting of triple negative breast cancer, non-small cell lung carcinoma, head and neck squamous cell cancer, classical Hodgkin lymphoma (cHL), bladder cancer, primary mediastinal B-cell lymphoma (PBMCL), urothelial carcinoma, microsatellite instability-high (MSI—H) solid tumors, mismatch repair deficient (dMMR) solid tumor, gastric or gastroesophageal junction (GEJ) adenocarcinoma, squamous cell carcinoma of the esophagus, cervical cancer, hepatocellular carcinoma, Merkel cell carcinoma, renal cell carcinoma, ovarian cancer, anal canal cancer, colorectal cancer, and melanoma.
72. The use of any of embodiments 54-71, wherein the patient is not administered an immune checkpoint inhibitor at the time of the administration of the CDK4/6 inhibitor.
73. The use of any of embodiments 38-72, wherein the patient is administered between about 22 and 26 hours prior to the first administration of the chemotherapy and again about 4 hours or less prior to the first administration of the chemotherapy.
74. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) determining whether the cancer is immunogenically susceptible to CDK4/6 inhibitor treatment;
   (ii) determining whether the patient can be administered a chemotherapy that induces an immune-response, for example an ICD-inducing chemotherapy, based on the cancer;
   (iii) and, if it is determined that the cancer is immunogenically susceptible to CDK 4/6 inhibitor treatment and that a chemotherapy that induces an immune-response can be administered, administering an effective amount of the chemotherapy in combination with an effective amount of the CDK4/6 inhibitor, wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone.
75. The use of embodiment 74, wherein the determination of whether the cancer is immunogenically susceptible to CDK4/6 inhibitor treatment comprises comparing a cancer tissue sample to those characterized in FIG. 7.
76. The use of embodiment 74, wherein the determination of whether the cancer is immunogenically susceptible to CDK4/6 inhibitor treatment comprises assessing the cancer according to FIG. 6.
77. The use of embodiment 74, wherein the determination of whether the cancer is immunogenically susceptible to CDK4/6 inhibitor treatment is according to the Galon immunoscore system.
78. The use of embodiment 74, wherein the determination of whether the cancer is immunogenically susceptible to CDK4/6 inhibitor treatment comprises assessing whether the cancer microenvironment has a sufficiently high level of major histocompatibility complex class I antigens available to initiate an effective immune response.
79. The use of embodiment 74, wherein the determination of cancer is immunogenically susceptible to CDK4/6 inhibitor treatment comprises assessing whether the cancer microenvironment has a sufficiently high level of major histocompatibility complex class II antigens available to initiate an effective immune response.
80. The use of embodiment 74, wherein the determination of whether the cancer is immunogenically susceptible to CDK 4/6 inhibitor comprises assessing whether the cancer microenvironment has a sufficiently high level of major histocompatibility complex class I and class II antigens available to initiate an effective immune response.
81. The use of any of embodiments 74-79, wherein the patient has a cancer that is immunogenically classified as a hot immune tumor.
82. The use of any of embodiments 74-79, wherein the patient has a cancer that is immunogenically classified as an altered-immunosuppressed tumor.
83. The use of embodiment 74-79, wherein the patient has a cancer that is a C2 IFN-γ Dominant class cancer, has a cancer microenvironment with a high IFN-γ Signature or a high Expanded Immune Signature, or has a cancer that is PD-L1 positive.
84. The use of embodiment 74, wherein the determination of whether the cancer is immunogenically susceptible to CDK4/6 inhibitor treatment comprises assessing whether the cancer microenvironment has a sufficiently high degree of T cell and cytotoxic T cell infiltration.
85. The use of embodiment 74, wherein the determination of whether the cancer is immunogenically susceptible to CDK4/6 inhibitor treatment comprises assessing whether the cancer has genomic instability and the microenvironment has presence of a pre-existing antitumor immune response.
86. The use of any of embodiments 74-85, wherein the CDK4/6 inhibitor administered is Compound I, or a pharmaceutically acceptable salt thereof
87. The use of any of embodiments 74-85, wherein the CDK4/6 inhibitor administered is Compound III, or a pharmaceutically acceptable salt thereof.
88. The use of any of embodiments 74-85, wherein the CDK4/6 inhibitor is administered about 24 hours or less prior to the administration of the chemotherapy.
89. The use of any of embodiments 74-85, wherein the CDK4/6 inhibitor is administered about 4 hours or less prior to the administration of the chemotherapy.
90. The use of any of embodiments 74-85, wherein the CDK4/6 inhibitor is administered about 30 minutes or less prior to the administration of the chemotherapy.
91. The use of any of embodiments 74-90, wherein the ICD-inducing chemotherapy is selected from the group consisting of cyclophosphamide, trabectedin, temozolomide, melphalan, dacarbazine, oxaliplatin, methotrexate, mitroxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations of thereof
92. The use of any of embodiments 74-90, wherein the chemotherapy is an immunogenic cell death (ICD)-inducing chemotherapy.
93. The use of any of embodiments 74-92, wherein the cancer is selected from the group consisting of triple negative breast cancer, non-small cell lung carcinoma, head and neck squamous cell cancer, classical Hodgkin lymphoma (cHL), bladder cancer, primary mediastinal B-cell lymphoma (PBMCL), urothelial carcinoma, microsatellite instability-high (MSI—H) solid tumors, mismatch repair deficient (dMMR) solid tumor, gastric or gastroesophageal junction (GEJ) adenocarcinoma, squamous cell carcinoma of the esophagus, cervical cancer, hepatocellular carcinoma, Merkel cell carcinoma, renal cell carcinoma, ovarian cancer, anal canal cancer, colorectal cancer, and melanoma.
94. The use of any of embodiments 74-93, wherein the patient is not administered an immune checkpoint inhibitor at the time of the administration of the CDK4/6 inhibitor.
95. The use of any of embodiments 74-93, wherein the patient is administered between about 22 and 26 hours prior to the first administration of the chemotherapy and again about 4 hours or less prior to the first administration of the chemotherapy.
96. The use of any of embodiments 74-95, wherein the patient is not administered an immune checkpoint inhibitor at the time of the administration of the CDK4/6 inhibitor.
97. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) determining whether the cancer is IFN-γ dominant;
   (ii) determining whether the patient can be administered a chemotherapy that induces an immune-response;
   (iii) and, if it is determined that the cancer is IFN-γ dominant and that a chemotherapy that induces an immune-response can be administered, administering an effective amount of the chemotherapy in combination with an effective amount of the CDK4/6 inhibitor,
   wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone.
98. The use of embodiment 97, wherein the determination of whether the cancer is IFN-γ dominant is based on a cancer microenvironment having high M1/M2 polarization, strong CD8+ T-cell staining, and a high T-cell receptor diversity.
99. The use of embodiment 97, wherein the determination of whether the cancer is IFN-γ dominant is based on the Thorsson et al. Six Class Immune Signature score classification.
100. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) determining whether the cancer has a high IFN-γ signature;
   (ii) determining whether the patient can be administered a chemotherapy that induces an immune-response;
   (iii) and, if it is determined that the cancer has a high IFN-γ signature and that a chemotherapy that induces an immune-response can be administered, administering an effective amount of the chemotherapy in combination with an effective amount of the CDK4/6 inhibitor,
   wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone.
101. The use of embodiment 100, wherein the determination of whether the cancer has a high IFN-γ signature is based on the expression levels of the genes IDO1, CXCL10, CSCL9, HLA-DRA, STAT1, and IFN-γ in the tumor microenvironment.
102. The use of embodiment 100, wherein the determination of whether the cancer has a high IFN-γ signature is based on a high Ayers et al. IFN-γ Signature score.
103. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) determining whether the cancer has a high expanded immunological signature;
   (ii) determining whether the patient can be administered a chemotherapy that induces an immune-response;
   (iii) and, if it is determined that the cancer has a high expanded immunological signature and that a chemotherapy that induces an immune-response can be administered, administering an effective amount of the chemotherapy in combination with an effective amount of the CDK4/6 inhibitor,
   wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone.
104. The use of embodiment 103, wherein the determination of whether the cancer has a high expanded immunological signature is based on the expression levels of the genes CCL5, CD27, CD274, CD276, CD8A, CMKLR1, CXCL9, CXCR6, HLA-DRB1, HLA-DQA1, HLA-E, IDOL LAG3, NKG7, PDCD1LG2, PSMB10, STAT1, and TIGIT in the tumor microenvironment.
105. The use of embodiment 103, wherein the determination of whether the cancer has a high expanded immunological signature is based on the Ayers et al. Expanded Immune Signature score.
106. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) determining whether the cancer is a hot tumor;
   (ii) determining whether the patient can be administered a chemotherapy that induces an immune-response;
   (iii) and, if it is determined that the cancer is a hot tumor and that a chemotherapy that induces an immune-response can be administered, administering an effective amount of the chemotherapy in combination with an effective amount of the CDK4/6 inhibitor,
   wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone.
107. The use of embodiment 106, wherein the determination of whether the cancer is a hot tumor comprises comparing a cancer tissue sample to those characterized in FIG. 7.
108. The use of embodiment 106, wherein the determination of whether the cancer is a hot tumor comprises assessing the cancer according to FIG. 6.
109. The use of embodiment 106, wherein the determination of whether the cancer is a hot tumor is according to the Galon immunoscore system.
110. The use of embodiment 106, wherein the determination of whether the cancer is a hot tumor comprises assessing whether the cancer microenvironment has a sufficiently high level of major histocompatibility complex class I antigens available to initiate an effective immune response.
111. The use of embodiment 106, wherein the determination of whether the cancer is a hot tumor comprises assessing whether the cancer microenvironment has a sufficiently high level of major histocompatibility complex class II antigens available to initiate an effective immune response.
112. The use of embodiment 106, wherein the determination of whether the cancer is a hot tumor comprises assessing whether the cancer microenvironment has a sufficiently high level of major histocompatibility complex class I and class II antigens available to initiate an effective immune response.
113. The use of embodiment 106, wherein the determination of whether the cancer is a hot tumor comprises assessing whether the cancer microenvironment has a sufficiently high degree of T cell and cytotoxic T cell infiltration.
114. The use of embodiment 106, wherein the determination of whether the cancer is a hot tumor comprises assessing whether the cancer microenvironment has immune checkpoint activation selected from expression of programmed cell death protein 1 (PD-1) expression and cytotoxic T lymphocyte-associated antigen 4 (CTLA4) expression.
115. The use of embodiment 106, wherein the determination of whether the cancer is a hot tumor comprises assessing whether the cancer microenvironment has T-cell immunoglobulin mucin receptor 3 (TIM3) expression and lymphocyte activation gene 3 (LAG3) expression.
116. The use of embodiment 106, wherein the determination of whether the cancer is a hot tumor comprises assessing whether the cancer microenvironment has impaired T-cell functions.
117. The use of embodiment 106, wherein the determination of whether the cancer is a hot tumor comprises assessing whether the cancer has genomic instability and the microenvironment has the presence of a pre-existing antitumor immune response.
118. Use of a compound selected from Compound I, Compound II, Compound III, Compound IV, Compound V, or a pharmaceutically acceptable salt thereof, in the manufacture of a medicament for cancer therapy to a selected patient or patient population in a manner that increases the progression free survival or overall survival of the patient or patient population comprising:
   (i) determining whether the cancer is PD-L1 positive;
   (ii) determining whether the patient can be administered a chemotherapy that induces an immune-response;
   (iii) and, if it is determined that the cancer is PD-L1 positive and that a chemotherapy that induces an immune-response can be administered, administering an effective amount of the chemotherapy in combination with an effective amount of the CDK4/6 inhibitor,
   wherein the CDK4/6 inhibitor is administered prior to administration of the chemotherapy, or alternatively, prior to and concurrently with the administration of the chemotherapy, and wherein the improvement in progression free survival or overall survival is in comparison to the progression free survival or overall survival based on administration of the chemotherapy alone.
119. The use of any of embodiments 97-118, wherein the CDK4/6 inhibitor administered is Compound I, or a pharmaceutically acceptable salt thereof
120. The use of any of embodiments 97-118, wherein the CDK4/6 inhibitor administered is Compound III, or a pharmaceutically acceptable salt thereof
121. The use of any of embodiments 97-118, wherein the CDK4/6 inhibitor is administered about 24 hours or less prior to the administration of the chemotherapy.
122. The use of any of embodiments 97-118, wherein the CDK4/6 inhibitor is administered about 4 hours or less prior to the administration of the chemotherapy.
123. The use of any of embodiments 97-118, wherein the CDK4/6 inhibitor is administered about 30 minutes or less prior to the administration of the chemotherapy.
124. The use of any of embodiments 97-123, wherein the ICD-inducing chemotherapy is selected from the group consisting of cyclophosphamide, trabectedin, temozolomide, melphalan, dacarbazine, oxaliplatin, methotrexate, mitroxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations of thereof
125. The use of any of embodiments 97-124, wherein the chemotherapy is an immunogenic cell death (ICD)-inducing chemotherapy.
126. The use of any of embodiments 97-125, wherein the cancer is selected from the group consisting of triple negative breast cancer, non-small cell lung carcinoma, head and neck squamous cell cancer, classical Hodgkin lymphoma (cHL), bladder cancer, primary mediastinal B-cell lymphoma (PBMCL), urothelial carcinoma, microsatellite instability-high (MSI—H) solid tumors, mismatch repair deficient (dMMR) solid tumor, gastric or gastroesophageal junction (GEJ) adenocarcinoma, squamous cell carcinoma of the esophagus, cervical cancer, hepatocellular carcinoma, Merkel cell carcinoma, renal cell carcinoma, ovarian cancer, anal canal cancer, colorectal cancer, and melanoma.
127. The use of any of embodiments 97-126, wherein the patient is not administered an immune checkpoint inhibitor at the time of the administration of the CDK4/6 inhibitor.
128. Use of a compound selected from Compound I, Compound II, Compound III, Compound IV, Compound V, or a pharmaceutically acceptable salt thereof, in the manufacture of a medicament for triple negative breast cancer therapy to a selected patient or patient population in a manner that increases the progression free survival or overall survival of the patient or patient population comprising:
   (i) determining if the triple negative breast cancer has a surrounding microenvironment that is favorable to immune modulation;
   (ii) determining if the chemotherapy regimen is capable of inducing an immune-mediated response, and
   (iii) if both (i) and (ii) are yes, administering an effective amount of the CDK 4/6 inhibitor, wherein the CDK4/6 inhibitor is administered prior to the administration of the chemotherapy or optionally prior to and concurrently with chemotherapy; and,
   wherein the increase in overall survival or progression free survival is in comparison to the overall survival or progression free survival based on administration of the chemotherapy alone.
129. The use of embodiment 128, wherein the determination of whether the cancer has a surrounding microenvironment that is favorable to immune modulation comprises comparing a cancer tissue sample to those characterized in FIG. 7.
130. The use of embodiment 128, wherein the determination of whether the cancer has a surrounding microenvironment that is favorable to immune modulation comprises assessing the cancer according to FIG. 6.
131. The use of embodiment 128, wherein the determination of whether the cancer has a surrounding microenvironment that is favorable to immune modulation is according to the Galon immunoscore system.
132. The use of embodiment 128, wherein the determination of whether the cancer has a surrounding microenvironment that is favorable to immune modulation comprises assessing whether the cancer microenvironment has a sufficiently high level of major histocompatibility complex class I antigens available to initiate an effective immune response.
133. The use of embodiment 128, wherein the determination of whether the cancer has a surrounding microenvironment that is favorable to immune modulation comprises assessing whether the cancer microenvironment has a sufficiently high level of major histocompatibility complex class II antigens available to initiate an effective immune response.
134. The use of embodiment 128, wherein the determination of whether the cancer microenvironment has a surrounding microenvironment that is favorable to immune modulation comprises assessing whether the cancer has a sufficiently high level of major histocompatibility complex class I and class II antigens available to initiate an effective immune response.
135. The use of any of embodiments 128, wherein the patient has a cancer that is immunogenically classified as a hot immune tumor.
136. The use of any of embodiments 128, wherein the patient has a cancer that is immunogenically classified as an altered-immunosuppressed tumor.
137. The use of embodiment 128, wherein the patient has a cancer that is a C2 IFN-γ Dominant class cancer, has a cancer with a high IFN-γ signature or a high expanded immune signature, or has a cancer that is PD-L1 positive, or a combination thereof
138. The use of embodiment 128, wherein the determination of whether the cancer has a surrounding microenvironment that is favorable to immune modulation comprises assessing whether the cancer microenvironment has a sufficiently high degree of T cell and cytotoxic T cell infiltration.
139. The use of embodiment 128, wherein the determination of whether the cancer has a surrounding microenvironment that is favorable to immune modulation comprises assessing whether the cancer microenvironment has immune checkpoint activation selected from expression of programmed cell death protein 1 (PD-1) expression and cytotoxic T lymphocyte-associated antigen 4 (CTLA4) expression.
140. The use of embodiment 128, wherein the determination of whether the cancer has a surrounding microenvironment that is favorable to immune modulation comprises assessing whether the cancer microenvironment has T-cell immunoglobulin mucin receptor 3 (TIM3) expression and lymphocyte activation gene 3 (LAG3) expression.
141. The use of embodiment 128, wherein the determination of whether the cancer has a surrounding microenvironment that is favorable to immune modulation comprises assessing whether the cancer microenvironment has impaired T-cell functions.
142. The use of embodiment 128, wherein the determination of whether the cancer has a surrounding microenvironment that is favorable to immune modulation comprises assessing whether the cancer has genomic instability and the microenvironment has the presence of a pre-existing antitumor immune response.
143. The use of any of embodiments 128-142, wherein the CDK4/6 inhibitor administered is Compound I, or a pharmaceutically acceptable salt thereof.
144. The use of any of embodiments 128-142, wherein the CDK4/6 inhibitor administered is Compound III, or a pharmaceutically acceptable salt thereof.
145. The use of any of embodiments 128-142, wherein the CDK4/6 inhibitor is administered about 24 hours or less prior to the administration of the chemotherapy.
146. The use of any of embodiments 128-142, wherein the CDK4/6 inhibitor is administered about 4 hours or less prior to the administration of the chemotherapy.
147. The use of any of embodiments 128-143, wherein the CDK4/6 inhibitor is administered about 30 minutes or less prior to the administration of the chemotherapy.
148. The use of any of embodiments 36-147, wherein the chemotherapy is gemcitabine and carboplatin.
149. Use of a compound selected from Compound I, Compound II, Compound III, Compound IV, Compound V, or a pharmaceutically acceptable salt thereof, in the manufacture of a medicament cancer therapy in selecting a patient or patient population for cancer therapy that includes the administration of a CDK 4/6 inhibitor with chemotherapy in a manner that reduces myelosuppression in a human patient receiving chemotherapy comprising:
   (i) determining if the cancer has a surrounding microenvironment that is not responsive to immune modulation;
   (ii) determining if the chemotherapy regimen induces chemotherapy-induced myelosuppression, and
   (iii) if both (i) and (ii) are yes, administering an effective amount of the CDK 4/6 inhibitor, wherein the CDK4/6 inhibitor is administered prior to the administration of the chemotherapy or optionally prior to and concurrently with chemotherapy; and,
   wherein the reduction in myelosuppression is in comparison to myelosuppression based on administration of the chemotherapy alone.
150. The use of embodiment 149, wherein the determination of whether the cancer has a surrounding microenvironment that is not favorable to immune modulation comprises comparing a cancer tissue sample to those characterized in FIG. 7.
151. The use of embodiment 149, wherein the determination of whether the cancer has a surrounding microenvironment that is not favorable to immune modulation comprises assessing the cancer according to FIG. 6.
152. The use of embodiment 149, wherein the determination of whether the cancer has a surrounding microenvironment that is favorable to immune modulation comprises assessing the cancer according to the Galon immunoscore system.
153. The use of embodiment 149, wherein the determination of whether the cancer has a surrounding microenvironment that is not favorable to immune modulation comprises assessing whether the cancer microenvironment has low level of major histocompatibility complex class I antigens.
154. The use of embodiment 149, wherein the determination of whether the cancer has a surrounding microenvironment that is not favorable to immune modulation comprises assessing whether the cancer microenvironment has a low level of major histocompatibility complex class II antigens.
155. The use of embodiment 149, wherein the determination of whether the cancer has a surrounding microenvironment that is not favorable to immune modulation comprises assessing whether the cancer microenvironment has a low level of major histocompatibility complex class I and class II antigens.
156. The use of embodiment 149, wherein the patient has a cancer that is immunogenically classified as a cold immune tumor.
157. The use of embodiment 149, wherein the patient has a cancer that has low IFN-γ expression in the tumor microenvironment, is not an IFN-γ dominant class cancer, has a cancer with a low IFN-γ signature or low expanded immune signature, or is PD-L1 negative.
158. The use of embodiment 149, wherein the determination of whether the cancer has a surrounding microenvironment that is not favorable to immune modulation comprises assessing whether the cancer microenvironment has a low degree of T cell and cytotoxic T cell infiltration.
159. The use of embodiment 149, wherein the determination of whether the cancer has a surrounding microenvironment that is not favorable to immune modulation comprises assessing whether the cancer microenvironment low programmed cell death protein 1 (PD-1) expression and low cytotoxic T lymphocyte-associated antigen 4 (CTLA4) expression.
160. The use of embodiment 149, wherein the determination of whether the cancer has a surrounding microenvironment that is not favorable to immune modulation comprises assessing whether the cancer microenvironment has low expression of T-cell immunoglobulin mucin receptor 3 (TIM3) and low expression of lymphocyte activation gene 3 (LAG3).
161. The use of any of embodiments 149-160, wherein the CDK4/6 inhibitor administered is Compound I, or a pharmaceutically acceptable salt thereof.
162. The use of any of embodiments 149-160, wherein the CDK4/6 inhibitor administered is Compound III, or a pharmaceutically acceptable salt thereof.
163. The use of any of embodiments 149-160, wherein the CDK4/6 inhibitor is administered about 24 hours or less prior to the administration of the chemotherapy.
164. The use of any of embodiments 149-160, wherein the CDK4/6 inhibitor is administered about 4 hours or less prior to the administration of the chemotherapy.
165. The use of any of embodiments 149-160, wherein the CDK4/6 inhibitor is administered about 30 minutes or less prior to the administration of the chemotherapy.
166. The use of any of embodiments 149-165, wherein the chemotherapy is selected from cyclophosphamide, trabectedin, temozolomide, melphalan, dacarbazine, oxaliplatin, methotrexate, mitroxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, cytosine arabinoside; and combinations of thereof
167. The use of any of embodiments 149-166, wherein the cancer is selected from the group consisting of triple negative breast cancer, non-small cell lung carcinoma, head and neck squamous cell cancer, classical Hodgkin lymphoma (cHL), bladder cancer, primary mediastinal B-cell lymphoma (PBMCL), urothelial carcinoma, microsatellite instability-high (MSI—H) solid tumors, mismatch repair deficient (dMMR) solid tumor, gastric or gastroesophageal junction (GEJ) adenocarcinoma, squamous cell carcinoma of the esophagus, cervical cancer, hepatocellular carcinoma, Merkel cell carcinoma, renal cell carcinoma, ovarian cancer, anal canal cancer, colorectal cancer, and melanoma.
168. The use of any of embodiments 149-166, wherein the cancer is not small-cell lung cancer.
169. The use of any of embodiments 36-147, wherein the CDK4/6 inhibitor is administered one or more times following the completion of chemotherapy treatment in a maintenance treatment regime, and wherein the chemotherapy is not administered at the time of CDK4/6 inhibitor administration.
170. The use of embodiment 169, wherein the CDK4/6 inhibitor is administered in an administration schedule selected from the group consisting of at least once a week, at least once every two weeks, at least once every three weeks, at least once a month, and at least once every six months.
171. The use of any of embodiments 36-147, wherein the CDK4/6 inhibitor is administered in combination with the chemotherapeutic one or more times following the completion of standard treatment in a chemotherapy dose reduced maintenance treatment regime, wherein the chemotherapy is administered at a lower dose than administered during the standard treatment.
172. The use of embodiment 171, wherein the CDK4/6 inhibitor and chemotherapy is administered in an administration schedule selected from the group consisting of at least once a week, at least once every two weeks, at least once every three weeks, at least once a month, at least once every six weeks, at least once every two months, at least once every three months, at least once every four months, at least once every five months, or at least once every six months.

### EXAMPLES

### Example 1. Trilaciclib Improves Overall and Progression Free Survival in Human Patients with Metastatic Triple Negative Breast Cancer Receiving Gemcitabine and Carboplatin

Study Design

A multicenter, randomized, open-label, Phase 2 study was developed to investigate the safety, tolerability, efficacy, and PK of once daily administration of trilaciclib (IV, 240 mg/m2) in combination with gemcitabine (IV, 1000 mg/m2) plus carboplatin (IV, AUC-2) (G/C) therapy for patients with metastatic TNBC (G1T28-04). Patients are randomly assigned (1:1:1 fashion) to 1 of 3 groups:

Group 1: G/C therapy (Days 1 and 8 of 21-day cycles);
Group 2: G/C therapy (Days 1 and 8) plus trilaciclib administered IV on Days 1 and 8 of 21-day cycles;
Group 3: G/C therapy (Days 2 and 9) plus trilaciclib administered IV on Days 1, 2, 8, and 9 of 21-day cycles;

Trilaciclib was administered intravenously prior to GC infusion.

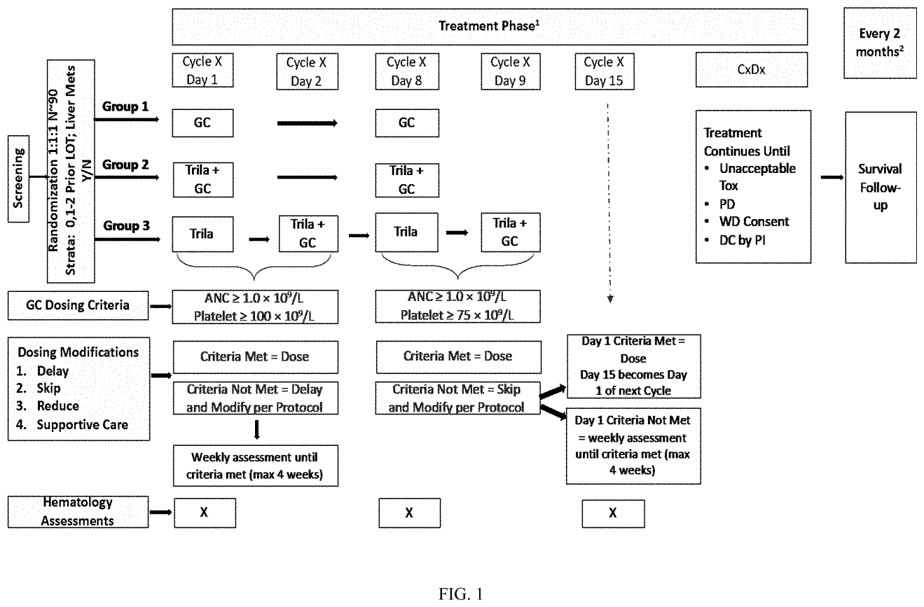

An overview of the study is provided is FIG. 1.

Adult patients (aged ≥18 years) with evaluable, biopsy-confirmed, locally recurrent or metastatic TNBC (mTNBC) were eligible for enrollment, provided tumors were estrogen and progesterone receptor negative by immunohistochemistry assessment (defined as <10% nuclei staining) and HER2 negative, according to American Society of Clinical Oncology/College of American Pathologists Clinical Practice Guidelines (i.e., non-overexpressing by local assessment of immunohistochemistry [0 or 1+] or fluorescent in situ hybridization [HER2/CEP17 ratio <2.0] or had an average HER2 gene copy number of <4 signals/nucleus by local assessment). The availability of diagnostic tumor tissue samples confirming TNBC was a prerequisite for enrollment. Hemoglobin levels must have been ≥9.0 g/dL in the absence of red blood cell (RBC) transfusion within 14 days prior to the first dose of trilaciclib, with an absolute neutrophil count (ANC)≥1.5×10⁹/L and a platelet count ≥100×10⁹/L. Patients were not eligible for inclusion if they had received >2 prior cytotoxic chemotherapy regimens for locally recurrent or mTNBC. Chemotherapy administered in the neoadjuvant/adjuvant setting was considered a line of therapy when <12 months had elapsed between the last treatment and disease recurrence. Patients must have had an Eastern Cooperative Oncology Group performance status of 0 or 1 and adequate kidney and liver function, as determined by laboratory tests of serum creatinine (≤1.5 mg/dL or creatinine clearance ≥60 mL/min), total bilirubin ≤1.5× upper limit of normal (ULN), and aspartate transaminase and alanine transaminase ≤2.5×ULN (or ≤5×ULN in the presence of liver metastases). With the exception of alopecia, resolution of non-hematological toxicities from prior treatment to grade ≤1 was required. Patients were not eligible for inclusion if they had malignancies other than TNBC within the 3 years prior to randomization, central nervous system metastases/leptomeningeal disease requiring immediate treatment, uncontrolled ischemic heart disease or symptomatic congestive heart failure, known history of stroke or cerebrovascular accident within 6 months prior to the first dose of trilaciclib, known serious active infection, or any other uncontrolled serious chronic disease or psychiatric condition that could affect patient safety, compliance, or follow-up. A washout period of 2 or 3 weeks was required for prior radiotherapy or cytotoxic chemotherapy, respectively, before study entry.

The study was designed and conducted in compliance with the principles of the Declaration of Helsinki and the Good Clinical Practice guidelines of the International Council for Harmonization. The study protocol and all study-related materials were approved by the institutional review board or independent ethics committee of each investigational site. Written informed consent was obtained from each patient prior to the initiation of study procedures.

Patients were randomly assigned to the following treatments given in 21-day cycles: group 1 was given gemcitabine and carboplatin on days 1 and 8 (chemotherapy only), group 2 was given trilaciclib before gemcitabine and carboplatin on days 1 and 8 (trilaciclib plus chemotherapy days 1 and 8), and group 3 was given trilaciclib only on days 1 and 8, and trilaciclib before gemcitabine and carboplatin on days 2 and 9 (trilaciclib days 1 and 8, trilaciclib plus chemotherapy days 2 and 9). Group 3 was included to test the hypothesis that a second dose of trilaciclib before chemotherapy could increase the proportion of haemopoietic stem and progenitor cells in transient arrest at the time of chemotherapy administration, thereby improving myelosuppression outcomes. Gemcitabine was administered at 1000 mg/m² and carboplatin at area under the concentration-time curve (AUC) 2 μg×h/mL, both as intravenous infusions. Trilaciclib 240 mg/m² was given as an intravenous infusion over 30 min (allowable range 25-35 min) before gemcitabine and carboplatin treatment. No dose modifications of trilaciclib were allowed.

Treatment cycles occurred consecutively without interruption, except when necessary to manage toxicities. If dose reductions were required for chemotherapy they occurred in the following order: first, the gemcitabine dose was reduced from 1000 mg/m² to 800 mg/m²; second, the carboplatin dose was reduced from AUC 2 μg×h/mL to AUC 1.5 μg×h/mL; third, either carboplatin or gemcitabine was discontinued and the other drug continued at the reduced dose; and finally, all study drugs were permanently discontinued. Dose reductions were allowed only once per cycle and were permanent.

Trilaciclib was administered only with GC therapy; if administration of chemotherapy was held or discontinued, trilaciclib was also held or discontinued. Study drug administration was continued until disease progression, unacceptable toxicity, withdrawal of consent, or discontinuation by the investigator, whichever occurred first.

Per protocol, samples were collected for hematological laboratory assessment on days 1, 8, and 15 of each 21-day cycle, regardless of treatment group. If the start of a subsequent cycle was delayed, laboratory assessments were done weekly (e.g., days 22, 29, 36, and so on) until the patient was able to start the next cycle or discontinued chemotherapy permanently. Unscheduled laboratory assessments were permitted as clinically indicated. The use of prophylactic growth factors, including granulocyte-colony stimulating factor (G-CSF) and granulocyte-macrophage colony-stimulating factor (GM-CSF) was not permitted during cycle 1. Otherwise, supportive care, including transfusions, was allowed as needed throughout the treatment period. Platelets were transfused at a threshold of 10,000 per μL or less or a platelet count less than 50,000 per μL (100,000 per μL for central nervous system or ocular bleeding). Patients with a hemoglobin concentration of less than 8.0 g/dL or with symptomatic anemia could be treated with red blood cell transfusions at the investigator's discretion. The percentage of patients receiving red blood cell trans-fusions and the number of red blood cell transfusions received over time was analyzed from on or after week 5 and from day 1 on study as part of a sensitivity analysis.

Assessment of antitumor response was performed by the investigator according to Response Evaluation Criteria in Solid Tumors (RECIST) version 1.1. For tumor assessment, computed tomography or magnetic resonance imaging was completed at screening and at protocol-specified intervals (every 9 weeks for the first 6 months, then every 12 weeks thereafter) until disease progression, withdrawal of consent, or receipt of subsequent anticancer therapy. Bone and brain scans were required at screening. Alternative imaging modalities could be used for follow-up assessments of bone lesions; brain scans were only repeated as part of tumor assessment if brain metastases were present.

To assess the ability of T cells to produce cytokines, whole blood was stimulated with 5 μg/mL staphylococcal enterotoxin B overnight (15-18 h) in the presence of Brefeldin A. Cells were processed and labelled with fluorophore-labelled antibodies against IFN-γ, IL-17A, CD3, and CD8 and assessed by flow cytometry (BD FACSCalibur and FACSCanto II clinical cell analysers; BD Biosciences, Franklin Lakes, N.J., USA) by Covance Central Laboratory Services (Indianapolis, Ind., USA and Geneva, Switzerland). Flow cytometry data were analysed by Fios Genomics.

Using two established signatures (Prosigna Breast Cancer Prognostic Gene Signature Assay [PAM50] and Lehmann triple-negative breast cancer type 1-4), patient tumors were characterized as CDK4/6 independent, dependent, or indeterminate. Because triple-negative breast cancer is predominantly a functionally CDK4/6-independent disease, despite a genomic retinoblastoma inactivation rate of only 20%, these signatures were chosen to provide a more comprehensive analysis of CDK4/6 sensitivity. Using the PAM50 signature, CDK4/6 independence correlates with basal-like tumors. Because their reliance on the CDK4/6 pathway for proliferation is either unknown or heterogeneous, the remaining PAM50 signature groups (including HER2, normal-like, luminal A, and luminal B) are categorized as CDK4/6 indeterminate. Conversely, using the Lehmann signature, CDK4/6 dependence is closely correlated with luminal-androgen receptor tumors, whereas the remaining Lehmann signature groups (including basal-like and mesenchymal) are categorized as CDK4/6 indeterminate for the same reasoning as outlined for the PAM50 signature.

Safety was monitored continuously throughout the study, from provision of informed consent until 30 days after the last dose of study treatment. Safety assessments included analyses of treatment duration and dose modifications, assessments of treatment-emergent adverse events and serious treatment-emergent adverse events, infusion-related reactions, laboratory safety assessments, vital signs, physical examination, and electrocardiography. Treatment-emergent adverse events were summarized according to grade (Common Terminology Criteria for Adverse Events [CTCAE] version 4.03) and association with study drug. Serious adverse events were defined as any untoward medical occurrence that, at any dose, results in death, a life-threatening event (i.e., the patient is at risk of death at the time of the event), inpatient admission to hospital or extension of current hospital admission, persistent or significant disability or incapacity, or a congenital anomaly or birth defect.

Because the primary toxicity of chemotherapy is myelosuppression (which trilaciclib was hypothesized to reduce), several hematological parameters were assessed across multiple hematopoietic lineages, including the incidence and severity of hematological adverse events, laboratory values (absolute neutrophil count, hemoglobin concentration, and platelet count), supportive care interventions (red blood cell and platelet transfusions, use of G-CSF), and dose intensity and incidence of gemcitabine and carboplatin dose reductions. Full details of all parameters are described further below.

Outcomes

The primary objective was to assess the safety and tolerability of trilaciclib given with chemotherapy; specific focused endpoints were detailed in the statistical analysis plan, which defined the primary endpoints as duration of severe neutropenia (severe neutropenia is defined as CTCAE grade 4, absolute neutrophil count <0.5×10⁹ cells per L) in cycle 1 and occurrence of severe neutropenia during the treatment period. Duration of severe neutropenia in cycle 1 was defined as the number of days from the date of the first absolute neutrophil count value of less than 0.5×10⁹ cells per L to the date of the first absolute neutrophil count value of 0.5×10⁹ cells per L or higher without observing absolute neutrophil count values of less than 0.5×10⁹ cells per L until the end of the cycle. Duration of severe neutropenia was set to zero days for patients who did not have severe neutropenia in cycle 1. The occurrence of severe neutropenia was a binary endpoint defined as those having one or more readings of absolute neutrophil count below 0.5×10⁹ cells per L during the treatment period. Both scheduled and unscheduled hematological laboratory results were included in the analysis of both primary endpoints. A clinically relevant level of 0.5×10⁹ cells per L was chosen for the primary analysis on the basis of the clinical link between severe neutropenia and an increased risk of infection and morbidity and mortality.

Key secondary endpoints included the occurrence of red blood cell transfusions on or after week 5, G-CSF administrations, platelet transfusions, and overall survival. The exclusion of red blood cell transfusions before week 5 on study was based on the half-life of red blood cells (approximately 8-9 weeks) and to ensure that analyses of potential benefit were not confounded by the residual impact of previous treatment. Occurrence of red blood cell and platelet transfusions was a binary endpoint (yes or no) and the total number of transfusions was a count endpoint (number of transfusions with a unique start date). Overall survival was calculated as the time (in months) from the date of randomization to the date of death due to any cause.

Supportive secondary antitumor activity endpoints are the proportion of patients who achieved an objective response (defined as a confirmed complete or partial response), duration of response, and progression-free survival. The clinical benefit rate was calculated using data from any patient who had a complete or partial response at any time after treatment or stable disease for 24 weeks or longer; if a patient did not have a complete or partial response and duration of stable disease was indeterminate, they were considered not evaluable. Progression-free survival was defined as the time (in months) from the date of randomization until the date of radiologically confirmed disease progression or death due to any cause, whichever came first.

Statistical Analysis

Specific endpoints across the trilaciclib development program and prespecified in the statistical analysis plan was used to show superiority of group 3 over group 1 with 90% power for at least one primary endpoint (either duration of severe neutropenia in cycle 1 or occurrence of severe neutropenia). An equally-weighted Bonferroni procedure was used to maintain the overall two-sided type I error rate at 0.05 and calculated that 64 patients (32 per group) were needed to detect a 3 day reduction of duration of severe neutropenia in cycle 1 with a common SD of 2.5 days or a 41 percentage point absolute reduction in the proportion of patients with severe neutropenia (i.e., 45% for the group 1 and 4% for group 3). Assuming a 5% attrition rate, we needed 102 patients in total (34 per group).

A non-parametric analysis of covariance was used to assess treatment group differences for duration of severe neutropenia in cycle 1 using stratification factors and treatment as fixed effects, with baseline absolute neutrophil count value as a covariate. For occurrence of severe neutropenia, G-CSF administration, red blood cell transfusions on or after week 5, and platelet transfusions, a modified Poisson regression model was used to assess the treatment effect. The model included the same fixed terms as used for duration of severe neutropenia, with baseline absolute neutrophil count as the covariate for severe neutropenia and G-CSF administration analyses, baseline hemoglobin concentration as the covariate for red blood cell transfusion analysis and baseline platelet count as the covariate for platelet transfusion analysis. Duration of treatment (in weeks) was adjusted in this model. From day 1 on study, the percentage of patients receiving red blood cell transfusions and the number of red blood cell transfusions over time was analyzed as part of a sensitivity analysis. For the number of red blood cell transfusions on or after week 5 and platelet transfusions, a negative binomial regression model was used to assess treatment effect. The model included the same fixed terms as used for duration of severe neutropenia, with baseline hemoglobin as the covariate for red blood cell transfusion analysis and baseline platelet count as the covariate for platelet transfusion analysis. Duration of treatment (in weeks) was adjusted in this model. The number of all-cause dose reductions was analyzed using a negative binomial regression model, which included only stratification factors and treatment as fixed effects, and was adjusted for the number of cycles. The family-wise type I error rate of 0.025 (one-sided) was controlled across the primary and key secondary myelosuppression endpoints using a Hochberg-based gatekeeping procedure. Model-based point estimates are reported along with 95% CIs.

Treatment group differences in objective response were analyzed by use of an exact Cochran-Mantel-Haenszel method accounting for the stratification factors. The 95% CIs for the proportion of patients who achieved an objective response was calculated using the exact Clopper-Pearson method. For time-to-event variables, such as duration of response, progression-free survival, and overall survival, the Kaplan-Meier method was used to estimate the median time and its 95% CI. Treatment group differences were tested using the stratified log-rank test to account for the stratification factors. Hazard ratios (HRs) and their associated 95% CIs were calculated from the Cox proportional hazards model, with treatment and stratification factors as fixed effects.

The statistical analysis plan prespecified the primary statistical comparison for the primary and key secondary endpoints to be between group 3 and group 1, and prespecified secondary comparisons were to be between group 2 and group 1, and between the combined trilaciclib groups and group 1.

Activity analyses were conducted using the intention-to-treat (ITT) population on the basis of the assigned treatment for myelosuppression and antitumor activity endpoints, with the exception of tumor response endpoints (objective response and clinical benefit), which were analyzed in patients who received at least one dose of study drug, had measurable target lesions at the baseline tumor assessment, and either had at least one tumor assessment after treatment (or no tumor assessment after treatment but had clinical progression as noted by the investigator) or had died due to disease progression before their first tumor scan after treatment (response evaluable population). Duration of survival follow-up was calculated from date of randomization to date of death or the last contact date as of activity data cutoff, which is specified throughout. Safety analyses included all patients who received at least one dose of study medication. The stratification factors (number of previous lines of systemic therapy and liver involvement) were adjusted in statistical models.

Prespecified subgroup analyses was performed for progression-free survival and overall survival to assess consistency of treatment effect (i.e., age group, race, liver involvement, country, ECOG performance status, number of previous lines of therapy, BRCA classification, and histological triple-negative breast cancer classification).

To address the theoretical risk that trilaciclib could decrease antitumor activity in patients with CDK4/6 dependent tumors by arresting CDK4/6-dependent tumor cells during chemotherapy, an additional prespecified subgroup analysis of the antitumor activity endpoints (objective response, progression-free survival, and overall survival) using two established signatures (PAM50 and Lehmann triple-negative breast cancer type 1-4) was performed to characterize patient tumors as CDK4/6 independent, dependent, or indeterminate.

A post-hoc analysis of antitumor activity endpoints (objective response, progression-free survival, and overall survival) was performed based on the overall median number of cycles patients received during the study. The analysis was done in patients grouped according to whether they had received 1-7 cycles or more than 7 cycles.

Results

142 patients were screened, and 102 eligible patients were randomly assigned to the chemotherapy alone group (group 1; n=34), the trilaciclib plus chemotherapy group (group 2; n=33), or in the trilaciclib (day 1 and 8), trilaciclib plus chemotherapy (day 2 and 9) group (group 3; n=35; ITT population). Of these, 98 (96%) patients received at least one dose of study drug (safety analysis population). Baseline demographic characteristics were similar between the treatment groups, which are disclosed in Table 3. 38 (37%) of 102 patients had received one or two previous lines of chemotherapy, and 26 (25%) had liver metastases.

TABLE 3
Demographics and Baseline Disease Characteristics

| | | Group 2 GC + | Group 3 GC + | |
|---|---|---|---|---|
| | Group 1 GC | Trilaciclib | Trilaciclib | |
| | (Day 1/8) | (Day 1/8) | (Day 1/2/8/9) | Total |
| | (n = 34) | (n = 33) | (n = 35) | (n = 102) |
| Sex | | | | |
| Female | 34 (100%) | 32 (97.0%) | 35 (100%) | 101 (99.0%) |
| Male | 0 | 1 (3.0%)* | 0 | 1 (<1.0%) |
| Median age, years | 55 (32-86) | 55 (34-78) | 58 (38-74) | 57 (32-86) |
| Age, years | | | | |
| 18-<65 | 26 (76.5%) | 24 (72.7%) | 26 (74.3%) | 76 (74.5%) |
| 65-75 | 6 (17.6%) | 7 (21.2%) | 9 (25.7%) | 22 (21.6%) |
| >75 | 2 (5.9%) | 2 (6.1%) | 0 | 4 (3.9%) |
| Race | | | | |
| White | 28 (82.4%) | 22 (66.7%) | 28 (80.0%) | 78 (76.5%) |
| Black or African | 5 (14.7%) | 7 (21.2%) | 2 (5.7%) | 14 (13.7%) |
| American | | | | |
| Asian | 0 | 2 (6.1%) | 4 (11.4%) | 6 (5.9%) |
| Other | 1 (2.9%) | 2 (6.1%) | 1 (2.9%) | 4 (3.9%) |
| Country | | | | |
| USA | 28 (82.4%) | 28 (84.8%) | 27 (77.1%) | 83 (81.4%) |
| Non-USA | 6 (17.6%) | 5 (15.2%) | 8 (22.9%) | 19 (18.6%) |
| Hormone receptor status | | | | |
| Oestrogen | | | | |
| <1% | 31 (91.2%) | 30 (90.9%) | 33 (94.3%) | 94 (92.2%) |
| 1-10% | 2 (5.9%) | 2 (6.1%) | 2 (5.7%) | 6 (5.9%) |
| Oestrogen negative, | 1 (2.9%) | 1 (3.0%) | 0 | 2 (2.0%) |
| other‡ | | | | |
| Progesterone | | | | |
| <1% | 29 (85.3%) | 29 (87.9%) | 34 (97.1%) | 92 (90.2%) |
| 1-10% | 4 (11.8%) | 3 (9.1%) | 1 (2.9%) | 8 (7.8%) |
| Progesterone | 1 (2.9%) | 1 (3.0%) | 0 | 2 (2.0%) |
| negative, | | | | |
| other‡ | | | | |
| ECOG PS | | | | |
| 0 | 15 (44.1%) | 17 (51.5%) | 21 (60.0%) | 53 (52.0%) |
| 1 | 19 (55.9%) | 16 (48.5%) | 14 (40.0%) | 49 (48.0%) |
| Liver involvement | 8 (23.5%) | 8 (24.2%) | 10 (28.6%) | 26 (25.5%) |
| Received neoadjuvant/ | 24 (70.6%) | 23 (69.7%) | 25 (71.4%) | 72 (70.6%) |
| adjuvant chemotherapy | | | | |
| Prior lines of therapy | | | | |
| IWRS | | | | |
| 0 | 21 (61.8%) | 22 (66.7%) | 21 (60.0%) | 64 (62.7%) |
| 1 or 2 | 13 (38.2%) | 11 (33.3%) | 14 (40.0%) | 38 (37.3%) |
| Clinical review | | | | |
| 0 | 18 (52.9%) | 19 (57.6%) | 17 (48.6%) | 54 (52.9%) |
| 1 | 11 (32.4%) | 11 (33.3%) | 14 (40.0%) | 36 (35.3%) |
| 2 | 5 (14.7%) | 3 (9.1%) | 4 (11.4%) | 12 (11.8%) |

Data are median (range) or n (%).
*One male patient was enrolled but later found to have an incorrect diagnosis of TNBC. His disease was pathologically confirmed as metastatic renal cell carcinoma.
‡Patients were determined by treating physician to have a diagnosis of TNBC, but not all pathology reports were available for review; includes 1 patient in Group 1 that was randomized but not treated.
ECOG PS = Eastem Cooperative Oncology Group performance status; GC = gemcitabine/carboplatin; IWRS = interactive web-response system; TNBC = triple-negative breast cancer.

The addition of trilaciclib to gemcitabine and carboplatin did not result in significant improvements in the predetermined, primary myelosuppression end points. During cycle 1, mean duration of severe neutropenia was 1 day (SD 2.4) in group 1, 2 days (3.5) in group 2, and 1.0 day (2.6) in group 3 (p=0.70). Severe neutropenia occurred in nine (26%) of 34 patients in group 1, 12 (36%) of 33 patients in group 2, and eight (23%) of 35 patients in group 3 (p=0.70; table 2). The number of red blood cell transfusions on or after week 5 per 100 weeks decreased in both trilaciclib groups (4.6 in group 1 vs 1.9 in group 2 and 1.6 in group 3; p=0.020). The red blood cell transfusion data collected from day 1 (sensitivity analysis) on study was similar to that observed when data before week 5 were excluded. No significant differences in the number of patients who were administered G-CSF or undergoing platelet transfusions was found.

TABLE 4
Myelosuppression Endpoints

| | | Group 2 GC + | Group 3 GC + | |
|---|---|---|---|---|
| | Group 1 GC | Trilaciclib | Trilaciclib | |
| | (Day 1/8) | (Day 1/8) | (Day 1/2/8/9) | |
| | (n = 34) | (n = 33) | (n = 35) | p-value^{‡} |
| Mean DSN in Cycle 1, days*^{§} | 1 (2.4) | 2 (3.5) | 1 (2.6) | 0.70** |
| Patients with SN*^{¶} | 9 (26.5%) | 12 (36.4%) | 8 (22.9%) | 0.70** |
| All-cause dose reductions, n per 100 | 14.1 | 11.8 | 13.3 | 0.98 |
| cycles^{#} | | | | |
| Patients with G-CSF administration^{¶} | 16 (47.1%) | 21 (63.6%) | 14 (40.0%) | 0.14 |
| Patients with RBC transfusion on/after | 12 (35.3%) | 11 (33.3%) | 8 (22.9%) | 0.075 |
| Week 5^{¶} | | | | |
| RBC transfusions on/after Week 5, | 4.6 | 1.9 | 1.6 | 0.020 |
| n per 100 weeks^{#} | | | | |
| Patients with platelet transfusions^{¶} | 4 (11.8%) | 3 (9.1%) | 6 (17.1%) | 0.98 |
| Platelet transfusions, n per 100 | 1.9 | 0.4 | 1.2 | 0.61 |
| weeks^{#} | | | | |

Data are mean (SD), n, or n (%).
Based on data as of Jul. 30, 2018.
*Primary endpoint;
**Multiplicity adjusted 1-sided p-value, all other p-values are 2-sided;
^{§}p-value obtained from a nonparametric analysis of covariance;
^{¶}p-value obtained from a modified Poisson model;
^{#}p-value was obtained from a negative binomial model.
^{‡}For comparison between Groups 3 and 1. Total number of all-cause dose reductions was the number of cycles with ≥1 dose reduction. If a patient did not have any dose reduction, they were assigned a value of 0.
DSN = duration of severe (Grade 4) neutropenia; GC = gemcitabine/carboplatin; G-CSF = granulocyte-colony stimulating factor; RBC = red blood cell; SD = standard deviation; SN = severe neutropenia.

As of the most recent evaluation of drug exposure (data cut Jun. 28, 2019), the number of patients with at least one carboplatin dose reduction was ten (33%) in the group 1, 13 (39%) in group 2, and 15 (43%) in group 3. For gemcitabine, 13 (43%) patients in group 1, 20 (61%) patients in group 2, and 17 (49%) patients in group 3 had at least one dose reduction. Adding trilaciclib to gemcitabine and carboplatin increased the duration of exposure and cumulative dose of gemcitabine and carboplatin compared with patients treated with gemcitabine and carboplatin alone. Median duration of treatment was 101 days (IQR 63-203 [median of four cycles]) in group 1, 161 days (77-287 [median of seven cycles]) in group 2, and 168 days (91-217 [median of eight cycles]) in group 3. The median cumulative dose of carboplatin was AUC 15 μg×h/mL (IQR 8-28) in group 1 versus AUC 24 μg×h/mL (IQR 10-40) in group 2 and AUC 22 μg×h/mL (IQR 15-34) in group 3. For gemcitabine, the median cumulative dose increased to 7306.2 mg/m² (IQR 4020.1-15138.9) in group 1, to 12000.0 mg/m² (IQR 5029.4-21882.7) in group 2 and 11800.1 mg/m² (IQR 7000.0-17446.9) in group 3. Despite a longer duration of gemcitabine and carboplatin for patients who received trilaciclib, hematological treatment-emergent adverse events occurred with similar frequency or less frequently in the trilaciclib groups as in the chemotherapy alone group.

At the most recent evaluation of safety data (data cut May 15, 2020), all but one patient (in group 3) had one or more treatment-emergent adverse events. For most patients, treatment-emergent adverse events were considered to be drug related. The most common treatment-emergent adverse events were anemia (22 [73%] of 34), neutropenia (21 [70%]), and thrombocytopenia (18 [60%]) in group 1; neutropenia (27 [82%]) of 33), thrombocytopenia (19 [58%]) and anemia (17 [52%]) in group 2; and neutropenia (23 [66%] of 35), thrombocytopenia (22 [63%]), and nausea (17 [49%]) in group 3. Febrile neutropenia occurred in one patient in group 1 and one patient in group 2. Serious treatment-emergent adverse events were reported in ten (33%) patients in group 1 and 11 (33%) in group 2, and four (11%) patients in group 3. All serious treatment-emergent adverse events occurred in two or fewer patients. There were 58 deaths in total; 25 in group 1 (disease progression [n=21], treatment-emergent adverse event [n=1; right ventricular failure considered unrelated to treatment], other [n=3]), 13 in group 2 (disease progression [n=11], other [n=2]), and 20 in group 3 (disease progression [n=19], other [n=1]). Overall, similar numbers of patients in each group reported a treatment-emergent adverse event that led to discontinuation of any study drug: ten (33%) in group 1, 14 (42%) in group 2, and 11 (31%) in group 3.

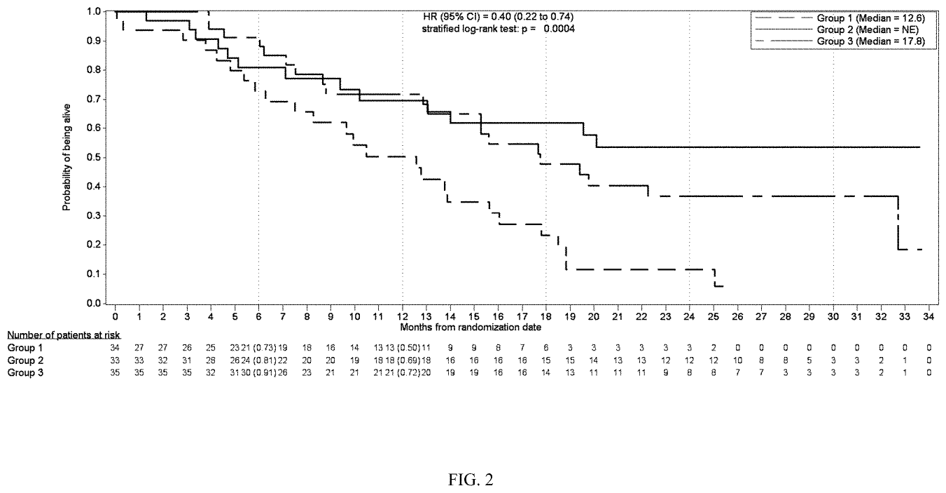

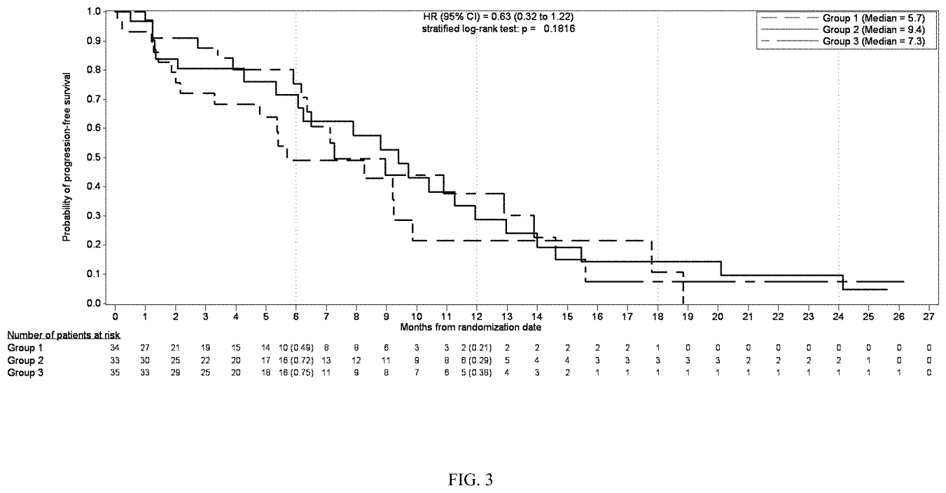

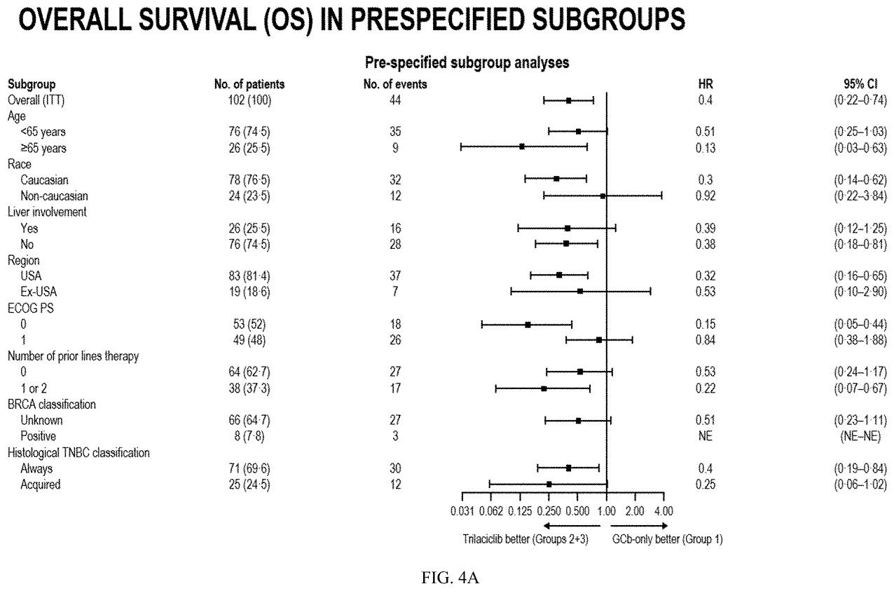

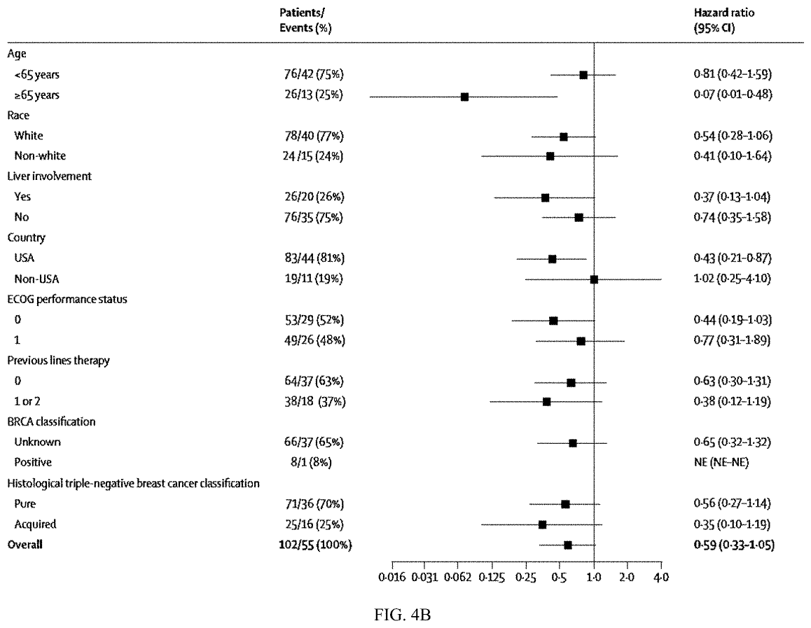

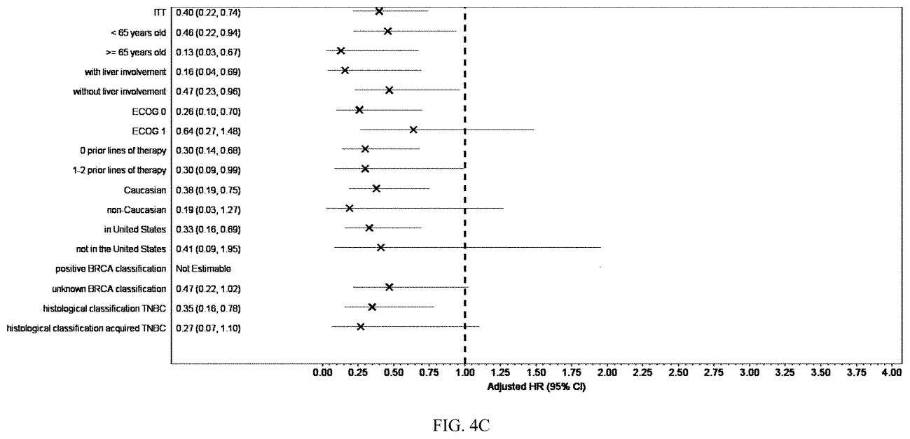

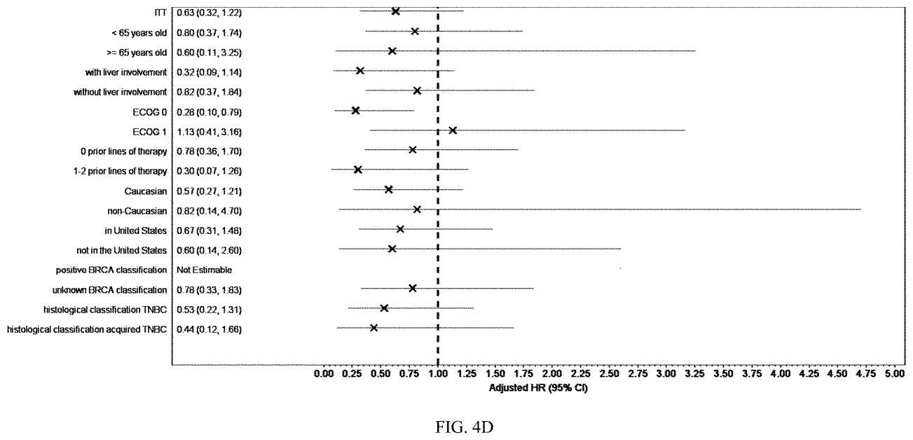

At the most recent evaluation of anti-tumor efficacy (data cut May 15, 2020), median follow-up was 8.4 months (IQR 3.8-15.6) for group 1, 14 months (5.5-26.8) for group 2, and 15.3 months (6.7-23.7) for group 3. Among patients evaluable for response, the proportion who achieved an objective response was 29.2% (7 of 24) in group 1 versus 50% (15 of 30) in group 2, and 38.7% (12 of 31) in group 3 (table 6). The proportion of patients who achieved clinical benefit, including stable disease for 24 weeks or longer, were 38% (nine of 24) in group 1, 57% (17 of 30) in group 2, and 43% (13 of 30) in group 3. Median progression-free survival was 9.4 months (IQR 5.3-13.0) in group 2, and 7.3 months IQR 6.2-13.9) in group 3, and 5.7 months (IQR 2.2-9.9) in group 1. The HR for both groups, analyzed separately against group 1, was 0.62 (95% CI 0.32-1.20; p=0.21) for group 2, and 0.63 (95% CI0.32-1.22; p=0.18) for group 3 (Table 3; FIG. 3). Patients enrolled in both trilaciclib groups had significant improvements in overall survival compared with those enrolled in group 1 (median not reached [IQR 9.4—not reached] and HR 0.31, 95% CI 0.15-0.63; p=0.0016 in group 2; 17.8 months [IQR 8.8-32.7] and HR 0.40, 95% CI 0.22-0.74; p=0.0004] in group 3 vs 12.6 months [IQR 5.8-17.8] in group 1 (Table 5; FIG. 2). The results of pooled subgroup analyses showed that the observed progression-free survival (FIG. 4B) and overall survival benefits (FIG. 4A) were consistent across subgroups as of the data cut of Jun. 28, 2019. Updated analyses between Group 1 and Group 3 are provided in FIG. 4C and FIG. 4D.

A prespecified assessment of objective response, progression-free survival, and overall survival across and within groups 1, 2, and 3 for tumors categorized as CDK4/6 independent, dependent, or indeterminate did not reveal any consistent trends favoring one tumor subtype over another.

TABLE 5
Antitumor Efficacy Results

| | | Group 2 GC + | Group 3 GC + | |
|---|---|---|---|---|
| | Group 1 GC | Trilaciclib | Trilaciclib | Group |
| | (Day 1/8) | (Day 1/8) | (Day 1/2/8/9) | 2 + 3 |
| Best overall response | | | | |
| Response evaluable | n = 24 | n = 30 | n = 31 | n = 61 |
| analysis set | | | | |
| CR | 0 | 0 | 0 | 0 |
| PR | 7 (29.2%) | 15 (50.0%) | 12 (38.7%) | 27 (44.3%) |
| SD | 11 (45.8%) | 9 (30.0%) | 14 (45.2%) | 23 (37.7%) |
| PD | 6 (25.0%) | 5 (16.7%) | 3 (9.7%) | 8 (13.1%) |
| NE^{†} | 0 | 0 | 1 (3.2%) | 1 (1.6%) |
| CBR ^{‡} | 18 (75.0%) | 24 (80.0%) | 26 (83.9%) | 50 (82.0%) |
| ORR | 7 (29.2%) | 15 (50.0%) | 12 (38.7%) | 27 (44.3%) |
| p-value* | | 0.14 | 0.36 | 0.19 |
| Progression-free survival | | | | |
| ITT | n = 34 | n = 33 | n = 35 | n = 68 |
| Patients with events | 19 (55.9%) | 22 (66.7%) | 18 (51.4%) | 40 (58.8%) |
| Median PFS, months (95% CI) | 5.7 (3.3-9.9) | 9.4 (6.1-11.9) | 7.3 (6.2-13.9) | 9.0 (6.4-11.3) |
| HR^{¶} | | 0.62 | 0.63 | 0.62 |
| p-value^{#} | | 0.21 | 0.18 | 0.13 |
| Overall survival | | | | |
| Patients with death | 25 (73.5%) | 13 (39.4%) | 20 (57.1%) | 33 (48.5%) |
| Median OS, months (95% CI) | 12.6 (6.3-15.6) | NE (10.2-NE) | 17.8 (12.9-32.7) | 19.8 (14.0-NE) |
| HR^{¶} | | 0.31 | 0.40 | 0.37 |
| p-value^{#} | | 0.0016 | 0.0004 | <0.0001 |
| Median duration of | 8.4 (3.8-15.6) | 14.0 (5.5-26.8) | 15.3 (6.7-23.7) | 15.3 (6.4-25.9) |
| follow-up, mondis (IQR)^{§} | | | | |

Data are median (IQR) or n (%).
Based on data as of May 15, 2020.
*The two-sided p-value was calculated using stratified exact Cochran-Mantel-Haenszel method to account for the stratification factors.
^{†}Includes patients who withdrew consent or did not have a tumour assessment during the study.
^{‡} Clinical benefit included any patient who had a CR or PR at any time post treatment or SD
^{#}Two-sided p-value was calculated using the stratified log-rank test to account for stratification factors.
^{¶}HR between the two treatments groups (trilaciclib vs GC only) was calculated from a Cox proportional hazard model in which treatment and the stratification factors of number of prior lines of therapy (0 vs 1-2) and presence of liver metastases (Yes or No) were included as fixed effects.
^{§}Duration of survival follow-up was calculated from first dose to the death date or the latest contact date as of May 15, 2020.
CI = confidence interval; CBR = clinical benefit rate; CR = complete response; GC = gemcitabine/carboplatin; HR = hazard ratio; IQR = interquartile range; ITT = intention-to-treat: NE = not evaluable; ORR = overall response rate: OS = overall survival; PD = progressive disease; PFS = progression-free survival; PR = partial response; SD = stable disease.

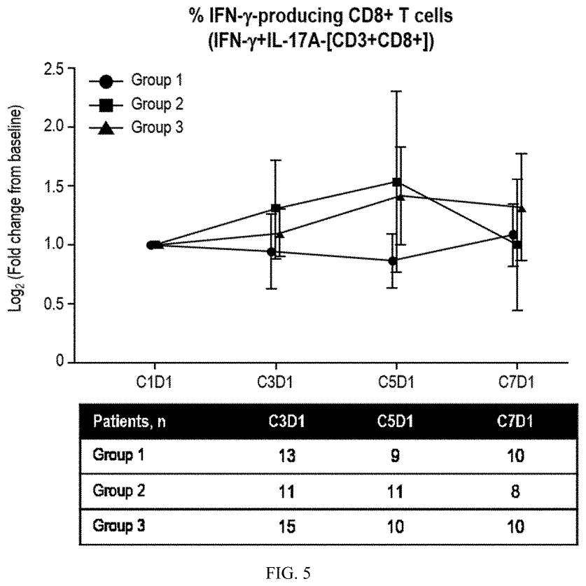

Although the addition of trilaciclib to gemcitabine and carboplatin did not preserve lymphocyte counts or enhance T-cell activation, in a prespecified analysis a higher frequency of CD8+ T cells producing IFN-γ after ex-vivo stimulation was observed in patients treated with gemcitabine and carboplatin plus trilaciclib than in patients in the group 1 (FIG. 5).

Archival tumor tissue from the TNBC diagnosis was evaluated using two different published RNA signatures: 1) CDK4/6 independent and variable CDK4/6 dependent buckets defined by PAM50 (see Prat et al., Response and survival of breast cancer intrinsic subtypes following multi-agent neoadjuvant chemotherapy. BMC Med. 2015; 13: 303. doi: 10.1186/s12916-015-0540-z, incorporated herein by reference); and 2) CDK4/6 dependent and variable CDK4/6 dependent buckets defined by Lehmann (see Lehmann et al., Identification of human triple-negative breast cancer subtypes and preclinical models for selection of targeted therapies. J Clin Invest. 2011; 121:2750-67. doi: 10.1172/JCI45014, incorporated herein by reference) (Table 6).

TABLE 6
Classifying CDK4/6 Dependency

| | Known | Variable | Known |
|---|---|---|---|
| Signature | Dependency | Dependency | Independence |
| PAM50 | | Other (HER2, | Basal-like |
| (Prat 2015) | | normal-like. | (52%) |
| | | LumA. LumB) (48%) | |
| Lehmann | LAR (36%) | Other | |
| TNBC type-4 | | (BL1, BL2, M) | |
| (Lehmann 2016) | | (64%) | |

The inclusion of trilaciclib in the chemotherapeutic regime did not antagonize chemotherapy efficacy in patients with TNBC with a variable or CDK4/6 dependent tumor, i.e., those classified as PAM50 Other (Her2, normal-like, LumA, LumB) or Lehmann TNBC type-4 LAR (Table 7). There were no differences in ORR/PFS/OS in known independent (basal like) patients across treatments.

TABLE 7
Trilaciclib with TNBC with Variable or CDK4/6 Dependent Tumors

| | PAM50 (Other) | | | | | | Lehmann (LAR) | | | | | |
|---|---|---|---|---|---|---|---|---|---|---|---|---|
| | ORR (%) | | PFS (mon) | | OS (mon) | | ORR (%) | | PFS (mon) | | OS (mon) | |
| Group 1 | 41.67 | 5/12 | 8.27 | 14 (8) | 10.17 | 14 (11) | 22.2 | 2/9 | 8.27 | 11 (5) | 9.67 | 11 (10) |
| Group 2. | 40 | 4/10 | 12.97 | 13 (4) | NA | 13 (5) | 40 | 4/10 | 12.12 | 12 (4) | NA | 12 (3) |
| Group 3 | 28.6 | 4/14 | 7.27 | 16 (8) | NA | 16 (6) | 11.1 | 1/9 | 5.9 | 9 (5) | 15.27 | 9 (5) |
| Group 1 + | 33.3 | 8/24 | 8.8 | 29 (12) | NA | 29 (11) | 26.3 | 5/19 | 8.8 | 21 (9) | NA | 21 (8) |
| Group 2 | | | | | | | | | | | | |

The inclusion of trilaciclib in the chemotherapeutic regime did not antagonize chemotherapy efficacy in patients with TNBC with a variable or CDK4/6 independent, i.e., those classified as PAM50 Basal-Like or Lehmann TNBCtype-4 Other (BL1, BL2, M) (Table 8).

TABLE 8
Trilaciclib with TNBC with Variable or CDK4/6 Independent Tumors

| | PAM50 (Basal-like) | | | | | | Lehmann (Other - BL1, BL2, M) | | | | | |
|---|---|---|---|---|---|---|---|---|---|---|---|---|
| | ORR (%) | | PFS (mon) | | OS (mon) | | ORR (%) | | PFS (mon) | | OS (mon) | |
| Group 1 | 30 | 3/10 | 5.7 | 14 (8) | 12.67 | 14 (7) | 46.5 | 6/13 | 5.7 | 17 (11) | 18.83 | 17 (8) |
| Group 2. | 56.3 | 9/16 | 7.9 | 17 (12) | 20.1 | 17 (6) | 56.3 | 9/16 | 6.23 | 18 (12) | 20.1 | 18 (8) |
| Group 3 | 50 | 6/12 | 10.9 | 16 (8) | 17.77 | 16 (7) | 52.9 | 9/17 | 10.9 | 23 (11) | 17.77 | 23 (8) |
| Group 1 + | 53.63 | 15/28 | 9.73 | 33 (20) | 20.1 | 33 (13) | 54.6 | 18/33 | 8.97 | 41 (23) | 20.1 | 41 (16) |
| Group 2 | | | | | | | | | | | | |

Results from a post-hoc analysis of antitumor effects according to median number of cycles (1-7 vs >7 cycles) are provided in Table 9. Across all three treatment groups, the proportion of patients who achieved an objective response was higher among those who received more than seven treatment cycles compared with those receiving seven cycles or fewer.

TABLE 9
Summary of Anti-tumor Effects by Cycle (Median splits, 1-7 vs. >7 cycles)

| | Patients with 1-7 cycles | | | | Patients with >7 cycles | | | |
|---|---|---|---|---|---|---|---|---|
| | Group 1 | Group 2 | Group 3 | | Group 1 | Group 2 | Group 3 | |
| | (G/C | (G/C + Trilaciclib | (G/C + Trilaciclib | Group | (G/C | (G/C + Trilaciclib | (G/C + Trilaciclib | Group |
| | D1 and | D1 and | D1/2 and | 2 + | D1 and | D1 | D1/2 and | 2 + |
| Category | 8 + SOC) | 8 + SOC) | 8/9 + SOC) | Group 3 | 8 + SOC) | and 8 + SOC) | 8/9 + SOC) | Group 3 |
| Objective response rate (CR + PR) | | | | | | | | |
| Response | 16 | 15 | 13 | 28 | 8 | 15 | 17 | 32 |
| evaluable | | | | | | | | |
| Analysis | | | | | | | | |
| Set | | | | | | | | |
| ORR | 3 | 2 (13.3%) | 1(7.7%) | 3 | 5 | 13 (86.7%) | 10 (58.8%) | 23 |
| | (18.8%) | | | (10.7%) | (62.5%) | | | (71.9%) |
| 95% CI | 4.0%, | 1.7%, 40.5% | 0.2%, 36.0% | 2.3%, | 24.5%, | 59.5%, 98.3% | 32.9%, 81.6% | 53.3%, |
| for ORR^{f} | 45.6% | | | 28.2% | 91.5% | | | 86.3% |
| p-value^{b} | | 0.70 | 0.68 | 0.47 | | 0.31 | 0.66 | 0.74 |

Progression-free survival

| | | | | | | | | |
|---|---|---|---|---|---|---|---|---|
| ITT | 19 | 17 | 17 | 34 | 11 | 16 | 18 | 34 |
| Patients | 14 | 9 (52.9%) | 7 (41.2%) | 16 | 4 | 10 (62.5%) | 11(61.1%) | 21 |
| with | (73.7%) | | | (47.1%) | (36.4%) | | | (61.8%) |
| events | | | | | | | | |
| [N (%)] | | | | | | | | |
| Median | 3.4 | 6.1 (1.3, 9.4) | 3.9 (2.7, 8.7) | 6.1 | 9.9 | 11.3 (6.2, 20.1) | 10.9 (6.5, 13.9) | 10.9 |
| PFS | (1.4, | | | (2.7, | (5.7, | | | (7.3, |
| (95% CI) | 5.4) | | | 8.7) | NE) | | | 13.9) |
| [months] | | | | | | | | |
| HR (95% | | 0.68 (0.28, 1.63) | 0.52 (0.19, 1.40) | 0.60 | | 0.89 (0.27, 3.01) | 1.17 (0.35, 3.90) | 1.06 |
| CI)^{d} | | | | (0.28, | | | | (0.35, |
| | | | | 1.27) | | | | 3.17) |
| p-value^{c} | | 0.45 | 0.11 | 0.18 | | 0.69 | 0.73 | 0.99 |

Overall survival

| | | | | | | | | |
|---|---|---|---|---|---|---|---|---|
| Patients | 16 | 9 (52.9%) | 10 (58.8%) | 19 | 3 | 2 (12.5%) | 4 (22.2%) | 6 |
| with | (84.2%) | | | (55.9%) | (27.3%) | | | (17.6%) |
| death [N | | | | | | | | |
| (%)] | | | | | | | | |
| Median | 8.3 | 10.2 (4.3, NE) | 13.0 (6.0, NE) | 12.9 | NE | 20.1 (20.1, NE) | 17.8 (15.6, NE) | 20.1 |
| OS (95% | (4.2, | | | (6.0, | (13.9, | | | (17.8, |
| CI) | 10.7) | | | NE) | NE) | | | NE) |
| [months] | | | | | | | | |
| HR (95% | | 0.52 (0.23, 1.20) | 0.36 (0.15, 0.88) | 0.45 | | 0.30 (0.03, 2.92) | 0.69 (0.14, 3.48) | 0.50 |
| CI)^{d} | | | | (0.23, | | | | (0.11, |
| | | | | 0.91) | | | | 2.23) |
| p-value^{c} | | 0.16 | 0.0013 | 0.0094 | | 0.28 | 0.54 | 0.30 |

CI = confidence interval, HR = hazard ratio, ORR = objective response rate, PFS = progression free survival, OS = overall survival.
Cycle 7 is the median number of total cycles that patients had during the treatment period.
^{a}Based on the data with cutoff date on 17 May 2019;
^{b}The two-sided p-value calculated using stratified exact CMH method to account for stratification factor;
^{c}The two-sided p-value calculated using the stratified log-rank test to account for the stratification factors;
^{d}The hazard ratio (HR) between the 2 treatments groups (trilaciclib versus GC only) was calculated from a Cox proportional hazard model in which treatment and the stratification factors (liver involvement [Yes or No] and the number of prior lines of anticancer therapy [0 vs 1-2]) were included as fixed effects;
^{f}The 95% CI for ORR was calculated using the exact Clopper-Pearson method.
Data as of May 15, 2019.

### Example 2: Ayers Immune Score Analysis

Tumor samples from patients participating in the clinical trial described in Example 1 where assayed by Q² Solutions (Morrisville, N.C.) to determine their Ayers Immune Scores according to Ayers et al., IFN-γ-related mRNA Profile Predicts Clinical Response to PD-1 Blockade, J Clin Invest. 2017127(8)2930-2940. The data was processed using RNA Access, and FPKM normalization prior to log 10 transformation and averaging was performed.

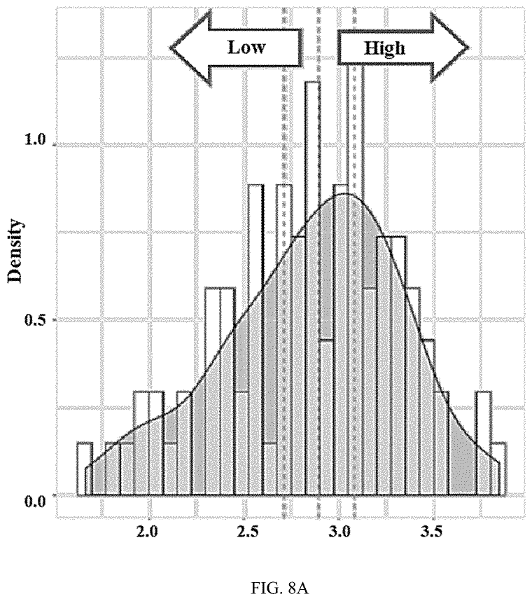

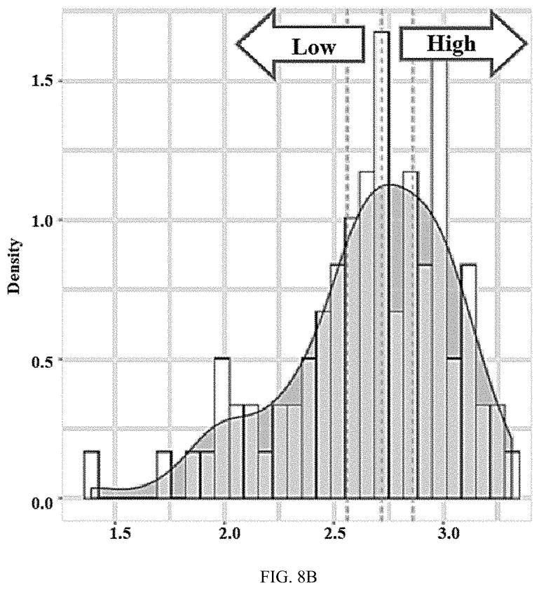

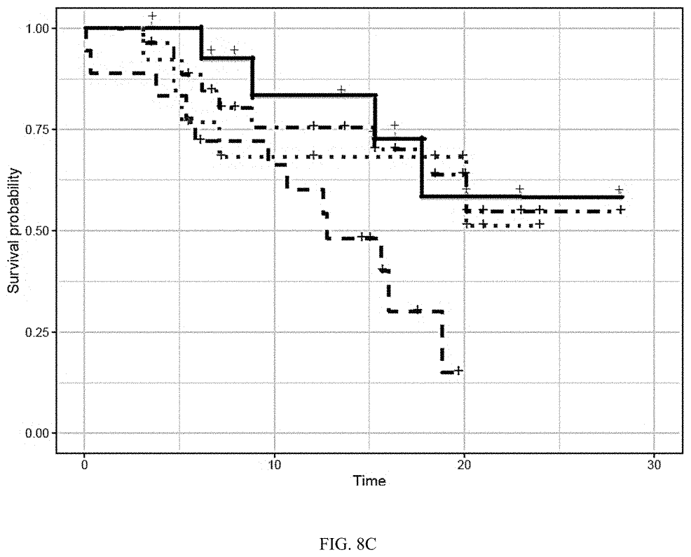

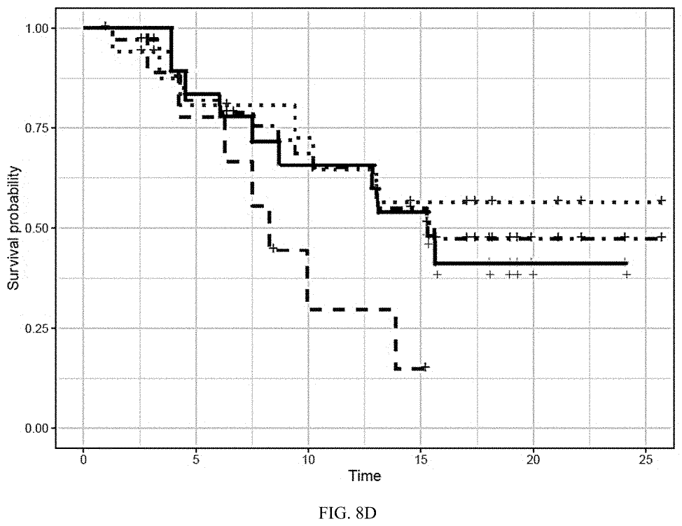

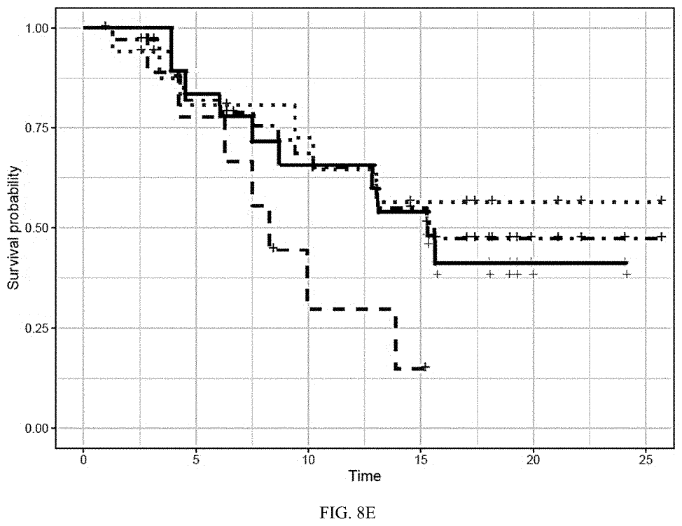

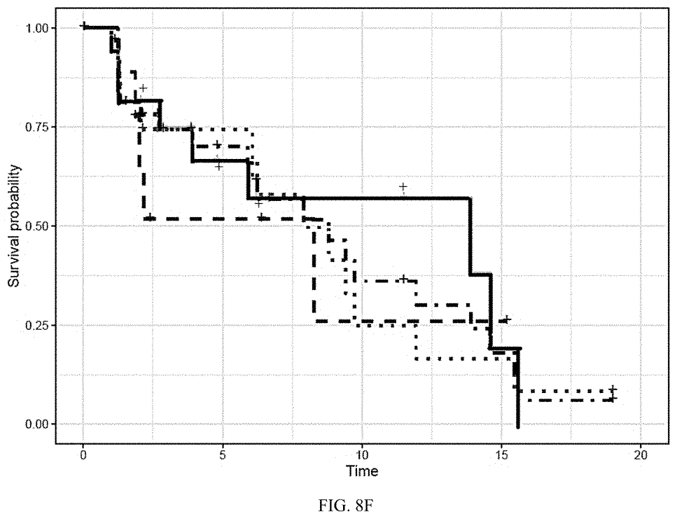

89 samples were analyzed. The calculated signature score for both the IFN-γ Signature and Expanded Immune Signature were unimodel in distribution, and the median score was used to define the “High” and “Low” categories. FIG. 8A shows the distribution of the Ayers' IFN-γ Signature across the 89 samples tested. FIG. 8B shows the distribution of the Ayers' Expanded Immune Signature across the 89 samples tested.

Survival and response rates between treatment groups within pre-defined immune response groups were determined using a series of pair-wise, two-group tests based on the data derived in the G1T28-04 clinical trial described in Example 1. Additionally, examination of differences in survival and response rates between different immune response categories within a single treatment group were analyzed. Results are provided in Table 10 and Table 11.

TABLE 10
IFN-y Signature Overall Survival and Progression-Free Survival

| | Overall Survival | | | PFS | | |
|---|---|---|---|---|---|---|
| | Group 1 | Group 2, 3 | | Group 1 | Group 2, 3 | |
| Subtype | Median | | p-value | Median | | p-value |
| High | 12.7667 | NR | 0.0194 | 5.7 | 11.267 | 0.0502 |
| | N = 18 | N = 27 | | N = 18 | N = 27 | |
| Low | 8.2667 | 15.6 | 0.0391 | 8.2667 | 8.8 | 0.6993 |
| | N = 10 | N = 35 | | N = 10 | N = 35 | |

NR = Not Reached
N = number of patients

TABLE 11
Expanded Immune Signature Overall Survival and Progression-Free Survival

| | Overall Survival | | | PFS | | |
|---|---|---|---|---|---|---|
| | Group 1 | Group 2, 3 | | Group 1 | Group 2, 3 | |
| Subtype | Median | | p-value | Median | | p-value |
| High | 12.7667 | NR | 0.0266 | 5.7 | 9.7333 | 0.0747 |
| | N = 17 | N = 34 | | N = 17 | N = 34 | |
| Low | 9.1 | NR | 0.0953 | 8.2667 | 9.4 | 0.7992 |
| | N = 11 | N = 35 | | N = 11 | N = 35 | |

NR = Not Reached
N = number of patients

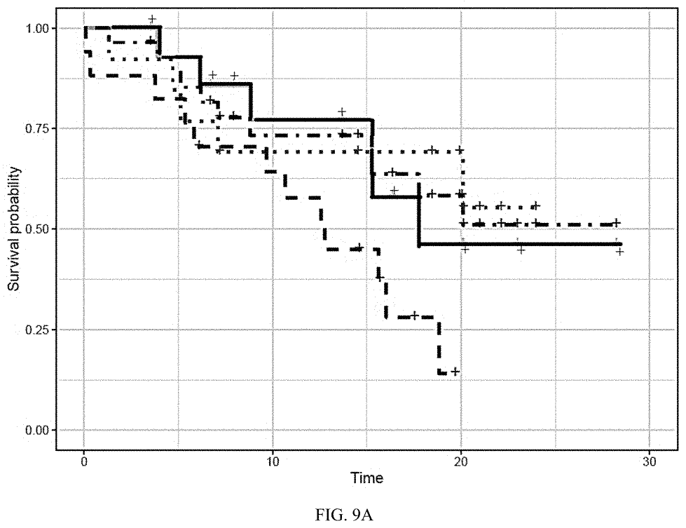

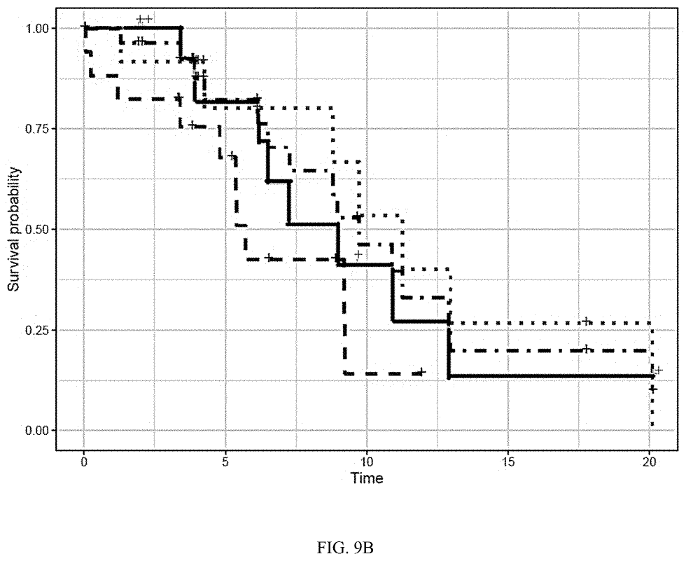

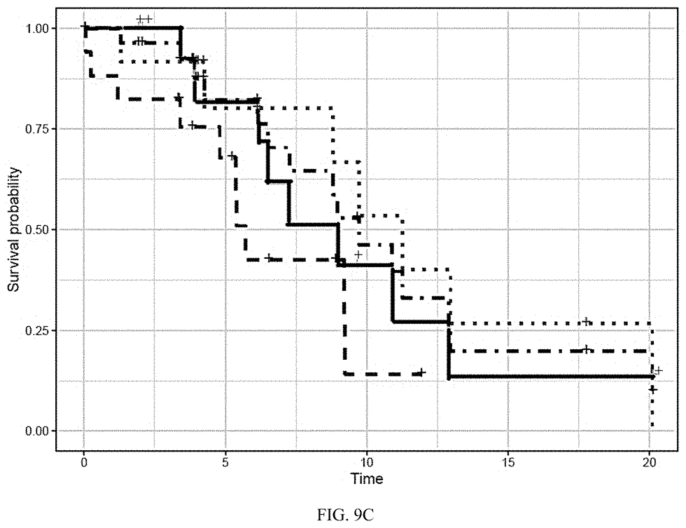

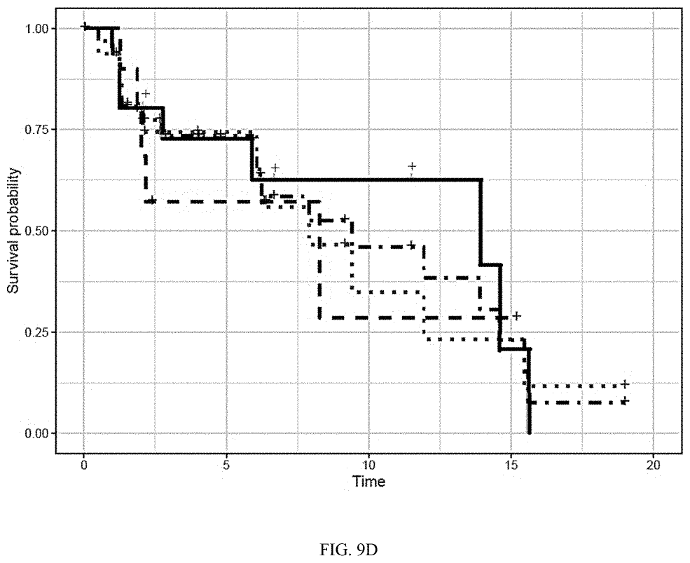

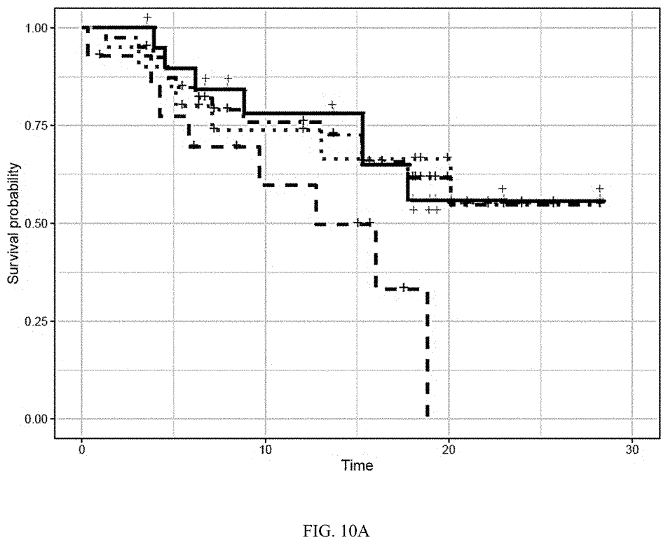

Kaplan-Meier curves were generated to visualize the overall survival and progression free survival among groups (see FIGS. 8C-8F and 9A-9D). The groups are divided as follows: Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and with respect to the Kaplan-Meier curve, Group 4 (Group 2+Group 3).

As shown, individuals with a TNBC which had a “high” IFN-γ Signature score and/or “high” Expanded Immune Signature score receiving trilaciclib as part of the treatment regimen had significantly improved overall survival compared to individuals with a TNBC which had a “high” IFN-γ Signature score and/or “high” Expanded Immune Signature score not receiving trilaciclib as part of the treatment regimen (p=0.0194; p=0.036, respectively).

### Example 3: Thorsson et al. Six Class Immune Classification Analysis

Tumor samples from patients participating in the clinical trial (Clinical trials.gov identifier NCT02978716) described in Example 1 where assayed by Q² Solutions (Morrisville, N.C.) to determine their Six Class Immune Classification as described in Thorsson V, et al. “The Immune Landscape of Cancer.” Immunity, vol 51, no. 2, 2018, pp. 812-830. doi: 10.1016/j.immuni.2018.03.023 (incorporated herein by reference in its entirety).

Briefly, a six-step procedure was implemented to apply the Thorsson et al. classification to 89 pre-treatment triple negative breast cancer samples secured in the clinical trial described in Example 1. Following RNAseq data procurement, the data was cleaned and homogenized by reconciling gene and sample names across data sources. Batch correction was performed in order to render the clinical trial-generated data and TCGA data comparable to ensure valid classification. In short, samples in the resulting TCGA expression data were randomly down-sampled to more closely reflect the abundance of PAM50 classes within the clinical trial data (previously derived, see Example 1, Table 7). Per-gene linear regression modelling on log 2 transformed, upper-quartile normalized expressions was used to estimate batch effects in the clinical trial data. These estimated batch effects were then removed from the expressions of clinical trial samples via arithmetic subtraction, resulting in a mean shift towards the TCGA samples.

PCA plots were used to examine the adequacy of this approach in reconciling the datasets. Prior to correction, Clinical trial samples and TCGA samples show clear batch effects via separation. The correction procedure discussed above diminishes the separations between the two groups of samples, rendering the two sets of expression data comparable. Following batch correction, the derived data was input into the Gibbs Immune Clustering software package (available at: https://github.com/CRI-iAtlas/ImmuneSubtypeClassifier) to classify the clinical trial samples with respect to the Thorsson's six category immune response schema. The script and its necessaries were downloaded, installed, and run on the batch-corrected clinical trial data.

The distribution of classes observed in the clinical trial samples are provided in Table 12.

TABLE 12
Six Class Immune Signature

| | | Number of |
|---|---|---|
| Group | Description | Samples |
| C1 | Wound Healing: High proliferation rate, | 27 |
| | high angiogenesis gene expression, TH2 | |
| | bias | |
| C2 | IFN-γ Dominant: High M1/M2 | 54 |
| | polarization, Strong CD8 and high | |
| | TCR diversity | |
| C3 | Inflammatory: | 4 |
| | Elevated Th17 and Th1 genes, low to | |
| | moderate proliferation, low aneuploidy | |
| | and overall somatic copy number | |
| | alterations. | |
| C4 | Lymphocyte Depleted: | 3 |
| | Prominent macrophage signature with | |
| | Th1 suppression and high M2 response | |
| C5 | Immunologically Quiet: | 0 |
| | Low lymphocyte response, High | |
| | macrophage response dominated by M2. | |
| C6 | TGF-β Dominant: | 2 |
| | Mixed tumour subgroup with high | |
| | TGF-β and lymphocytic infiltration. | |

No patients were classified as immunologically quiet (C5). This is not surprising as none of the training samples used by Thorsson et al. for breast cancers were classified as immunologically quiet in the original manuscript. In addition, due to the low prevalence of classes outside of C1 and C2, it was decided to exclude testing for C3 vs Not C3, C4 vs Not C4, and C6 vs Not C6.

Survival and response rates between treatment groups within pre-defined immune response groups were determined using a series of pair-wise, two-group tests based on the data derived in the G1T28-04 clinical trial described in Example 1. Additionally, examination of differences in survival and response rates between different immune response categories within a single treatment group were analyzed. Results are provided in Table 13.

TABLE 13
Six Class Immune Signature Overall Survival and Progression-Free Survival

| | Overall Survival | | | PFS | | |
|---|---|---|---|---|---|---|
| | Group 1 | Group 2, 3 | | Group 1 | Group 2, 3 | |
| Subtype | Median | | p-value | Median | | p-value |
| C2 IFN-γ | 12.7667 | NR | 0.036 | 9.2 | 10.9 | 0.2713 |
| Dominant | | | | | | |
| C2 Not | 10.3 | 13.0667 | 0.1885 | 5.4 | 9.4 | 0.4587 |
| IFN-γ | | | | | | |
| Dominant | | | | | | |

NR = Not Reached

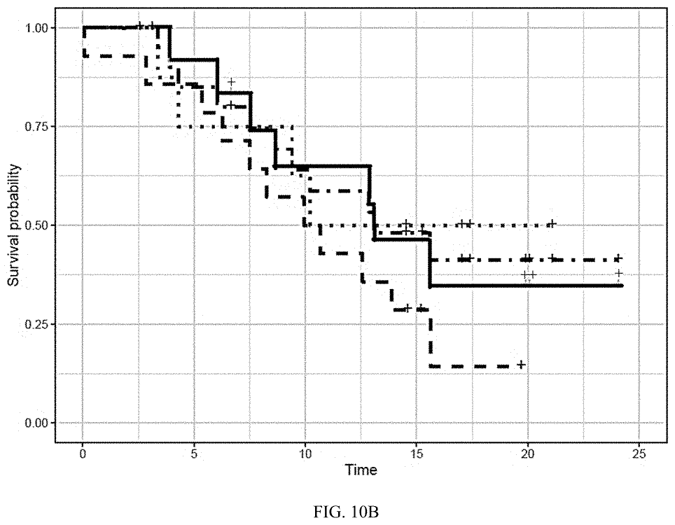

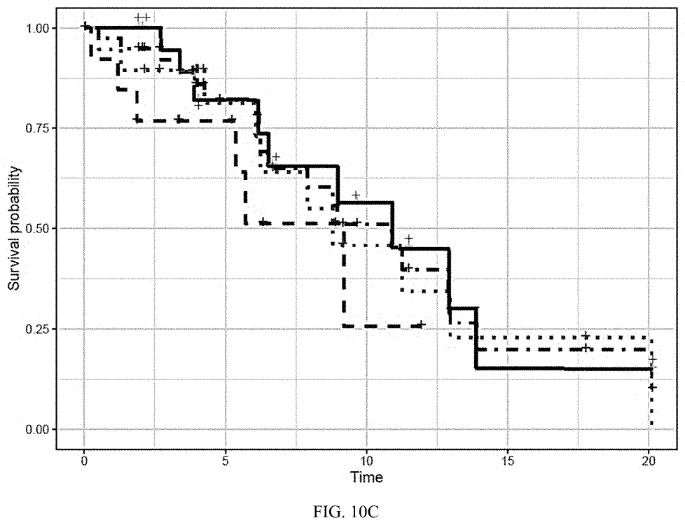

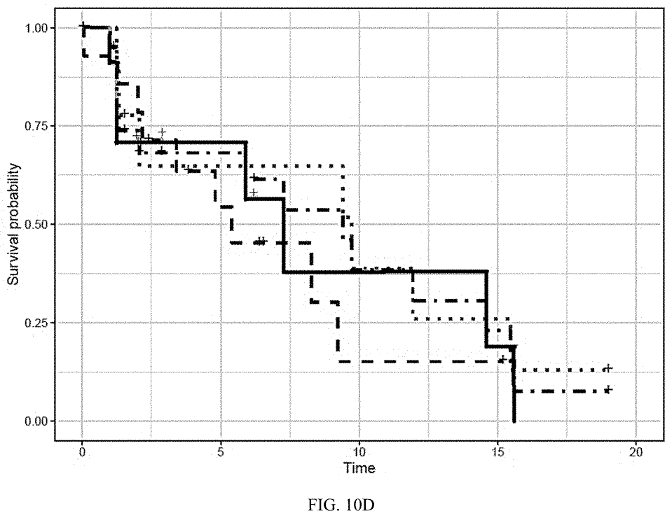

Kaplan-Meier curves were generated to visualize the overall survival and progression free survival among groups (see FIG. 10A-10D). The groups are divided as follows: Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle), and with respect to the Kaplan-Meier curved, Group 4 (Group 2+Group 3).

As shown, individuals with a TNBC classified as C2 IFN-γ Dominant receiving trilaciclib as part of the treatment regimen had significantly improved overall survival compared to individuals with a TNBC classified as C2 IFN-γ Dominant not receiving trilaciclib as part of the treatment regimen (p=0.036).

### Example 4: PD-L1 Tumor Status

Patient tumors from the G1T28-04 clinical trial described in Example 1 were characterized based on PD-L1 expression scored as negative or positive if <1% or ≥1% of the total tumor area contained PD-L1-labelled immune cells, respectively, using the Ventana SP142 assay. Association of PD-L1 expression with antitumor efficacy was assessed using proportional hazards regression. The groups are divided as follows: Group 1 (gemcitabine+carboplatin only on days 1 and 8 in a 21-day cycles), Group 2 (gemcitabine+carboplatin+trilaciclib on days 1 and 8 in a 21 day cycle), and Group 3 (gemcitabine+carboplatin on days 2 and 9+trilaciclib on days 1, 2, 8, and 9 in a 21-day cycle). Results are provided in Table 14.

TABLE 14
PD-Ll Positive Tumor Status Overall Survival
OVERALL SURVIVAL

| | PD-Ll Positive | | | | PD-L1 Negative | | | |
|---|---|---|---|---|---|---|---|---|
| | | | | Group 2 + | | | | Group 2 + |
| | Group 1 | Group 2 | Group 3 | Group 3 | Group 1 | Group 2 | Group 3 | Group 3 |
| | (n = 17) | (n = 16) | (n = 16) | (n = 32) | (n = 10) | (n = 10) | (n = 16) | (n = 26) |
| Patients | 14 | 7 (43.8%) | 7 (43.8%) | 14 | 8 (80%) | 4 (40%) | 10 | 14 |
| with | (82.4%) | | | (43.8%) | | | (62.5%) | (53.8%) |
| event | | | | | | | | |
| Median | 10.5 | 20.1 | 32.7 | 32.7 | 13.87 | NR | 17.77 | 17.77 |
| (KM, | | | | | | | | |
| month) | | | | | | | | |
| 95% CI | 6.27; | 10.2; NR | 15.27; NR | 17.67; NR | 12.57; NR | 9.4; NR | 12.87; NR | 13.07; NR |
| Median | 18.83 | | | | | | | |
| p value | | 0.025 | 0.019 | 0.005 | | 0.092 | 0.248 | 0.1.09 |
| (Wald | | | | | | | | |
| Test) | | | | | | | | |
| Hazard | | 0.35 | 0.33 | 0.34 | | 0.35 | 0.58 | 0.49 |
| Ratio | | | | | | | | |
| 95% CI | | 0.14; 0.87 | 0.13; 0.84 | 0.16; 0.73 | | 0.11; 1.18 | 0.23; 1.47 | 0.2; 1.17 |

NR = Not Reached

As shown above, patients with PD-L1 positive TNBC tumors receiving trilaciclib had a statistically significant overall survival than patients with PD-L1 positive TNBC tumors who did not receive trilaciclib (p=0.005).

### Example 5: Trilaciclib Administration Enhances T-Cell Expansion

A phase 2 study of carboplatin, etoposide, and atezolizumab with or without trilaciclib in patients with untreated extensive stage small cell lung cancer was initiated (Clinicaltrials.gov identifier NCT03041311). carboplatin comprising a 21-day induction phase and a 21-day maintenance phase. Four induction phase cycles were completed prior to the initiation of the maintenance phase cycle.

In the induction phase, patients received trilaciclib (240 mg/m2 diluted in 250 mL D5W or sodium chloride solution 0.9%) or placebo (250 mL of D5W or sodium chloride solution 0.9%) administered IV once daily on days 1 to 3 of a 21-day cycle of each etoposide/carboplatin/atezolizumab (E/P/A) therapy cycle (up to 4 cycles in total). The carboplatin dose was calculated using the Calvert formula [total carboplatin dose (mg)=(target AUC)×(GFR+25)] with a target AUC=5 (maximum 750 mg) IV over 30 minutes on day 1, and 100 mg/m2 etoposide administered IV over 60 minutes daily on days 1, 2, and 3 of each 21-day cycle. Atezolizumab (1200 mg) in 250 mL sodium chloride solution 0.9% was administered as an IV infusion on day 1 of each 21-day cycle in both the induction and maintenance phases. Atezolizumab was infused over 60 minutes for the first administration and, if tolerated, all subsequent infusions were delivered over 30 minutes. Atezolizumab was administered following the completion of administration of Compound I or placebo, etoposide, and carboplatin.

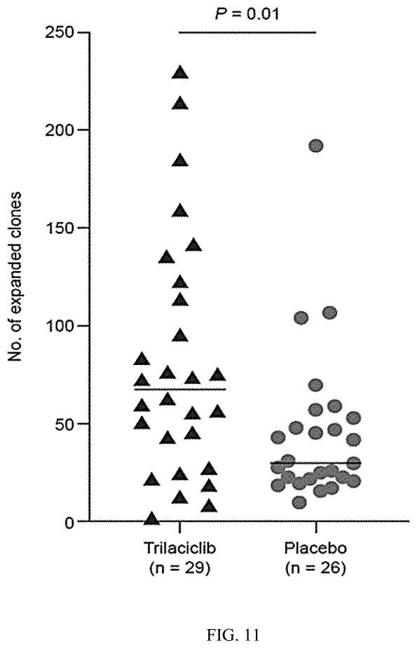

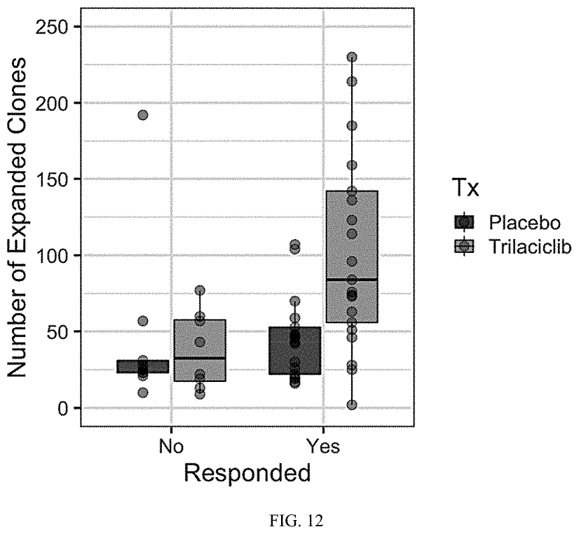

Analysis of the TCRB locus in blood samples was performed to identify biomarkers of immune response and immunomodulatory activity between the trilaciclib arm and the placebo arm. All samples were sequenced using a 1-rxn TCRB Assay. The number of expanded T-cell clones was determined by the differential abundance analysis of T-cell receptor β sequences in whole blood from patients at after induction and prior to starting maintenance versus baseline. Trilaciclib responders had more clonal expansion than Placebo responders (p=0.01) as well as more clonal expansion than Trilaciclib non-responders (p=0.006), suggesting that increased clonal expansion is a biomarker of both Trilaciclib MOA and clinical response (FIG. 11 and FIG. 12).

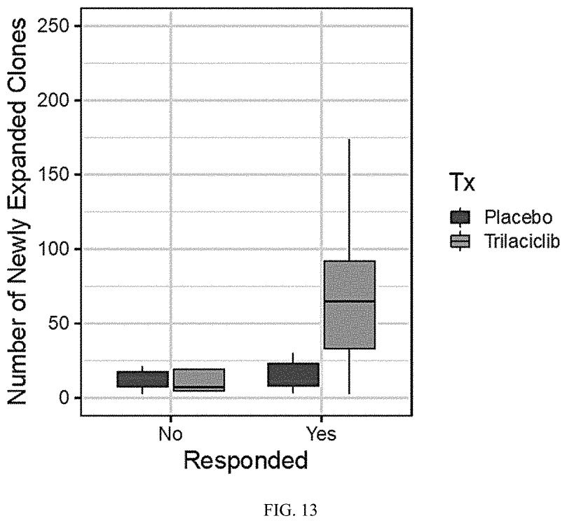

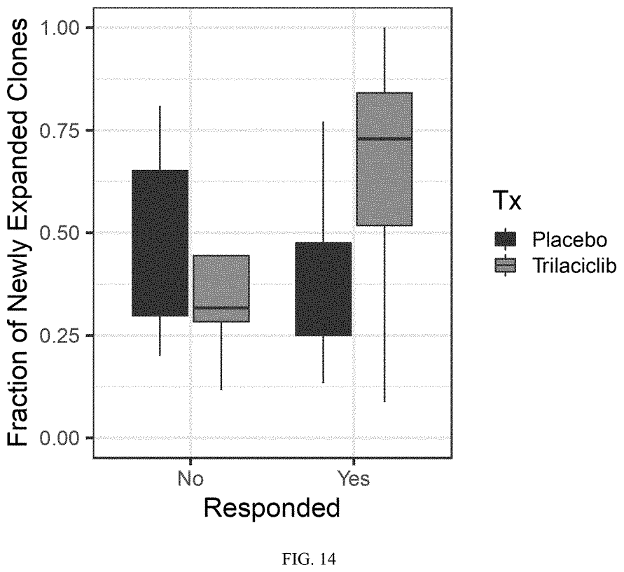

Additionally, responders receiving trilaciclib also generated more newly expanded clones (p=0.001, FIG. 13) and, strikingly, significantly increased the fraction of newly expanded clones versus all expanded clones when compared to patients that responded to treatment without receiving trilaciclib (p=0.001, FIG. 14). While stratification of patients below and above the median fractions of newly expanded versus all T-cell clones revealed a non-significant trend for patients with higher levels of peripheral T-cell clonal expansion to have longer overall survival OS (HR [95% CI]=0.56, P=0.10), a subgroup analysis (trilaciclib versus placebo) revealed that patients above the newly expanded clone fraction median had a significantly longer OS when receiving trilaciclib (HR=0.34; p=0.04) with similar but non-significant trends in PFS. Similar benefit was observed for median clonal expansion and median newly expanded clone stratification. Unlike the placebo, trilaciclib significantly increased the number and fraction of newly expanded clones demonstrating that the addition of trilaciclib to the etoposide, carboplatin, atezolizumab treatment regimen enhances the T-cell mediated anti-tumor response.

This specification has been described in reference to embodiments of the invention. However, one of ordinary skill in the art appreciates that various modification and changes can be made without departing from the scope of the invention as set forth in the claims below. Accordingly, the specification is to be regarded in an illustrative rather than restrictive sense, and all such modifications are intended to be included within the scope of the invention.

1. A method for selecting a patient or patient population for cancer therapy that includes the administration of a CDK 4/6 inhibitor with chemotherapy in a manner that increases the progression free survival or overall survival of the patient comprising:
(i) determining if the patient's cancer has a surrounding microenvironment that is favorable to immune modulation;
(ii) determining whether the chemotherapy regimen induces an immune-mediated response within the microenvironment, and if both (i) and (ii) are yes, then,
a. administering to the patient an effective amount of a CDK4/6 inhibitor having the structure: 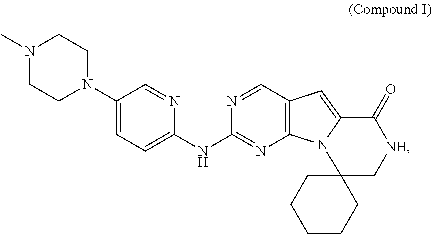 [SMILES: CN1CCN(c2ccc(Nc3ncc4cc5n(c4n3)C3(CCCCC3)CNC5=O)nc2)CC1]
or a pharmaceutically acceptable salt thereof, and,
b. administering to the patient an effective amount of the chemotherapy regimen to induce an immune-mediated response,
wherein the CDK4/6 inhibitor is administered prior to the administration of the chemotherapy; and,
wherein the increase in progression free survival or overall survival is in comparison to the progression free survival or overall survival based on administration of the chemotherapy regimen alone.

2. The method of claim 1, wherein the determination of whether the cancer has a surrounding microenvironment that is favorable to immune modulation comprises comparing a cancer tissue sample to those characterized in FIG. 7.

3. The method of claim 1, wherein the determination of whether the cancer has a surrounding microenvironment that is favorable to immune modulation comprises assessing the cancer according to FIG. 6.

4. The method of claim 1, wherein the determination of whether the cancer has a surrounding microenvironment that is favorable to immune modulation is based on the Galon immunoscore system.

5. The method of claim 1, wherein the determination of whether the cancer has a surrounding microenvironment that is favorable to immune modulation comprises assessing whether the cancer has a sufficiently high level of major histocompatibility complex class I antigens available to initiate an effective immune response.

6. The method of claim 1, wherein the determination of whether the cancer has a surrounding microenvironment that is favorable to immune modulation comprises assessing whether the cancer has a sufficiently high level of major histocompatibility complex class II antigens available to initiate an effective immune response.

7. The method of claim 1, wherein the determination of whether the cancer has a surrounding microenvironment that is favorable to immune modulation comprises assessing whether the cancer has a sufficiently high level of major histocompatibility complex class I and class II antigens available to initiate an effective immune response.

8. The method of claim 1, wherein the patient has a cancer that is immunogenically classified as a hot immune tumor.

9. The method of claim 1, wherein the patient has a cancer that is immunogenically classified as an altered-immunosuppressed tumor.

10. The method of claim 1, wherein the determination of whether the cancer has a surrounding microenvironment that is favorable to immune modulation comprises assessing whether the cancer (i) is an IFN-γ Dominant class cancer, (ii) has a microenvironment with a high IFN-γ Signature, (iii) has a high Expanded Immune Signature, (iv) is PD-L1 positive, or a combination thereof.

11. The method of claim 1, wherein the CDK4/6 inhibitor is administered about 4 hours or less prior to the administration of the chemotherapy.

12. The method of claim 1, wherein the chemotherapy is selected from the group consisting of cyclophosphamide, trabectedin, temozolomide, melphalan, dacarbazine, oxaliplatin, methotrexate, mitroxantrone, gemcitabine, 5-fluorouracil (5-FU), bleomycin, doxorubicin, daunorubicin, epirubicin, idarubicin, valrubicin, paclitaxel, cabazitaxel, docetaxel, topotecan, irinotecan, etoposide, carboplatin, cisplatin; bortezomib, vinblastine, vincristine, vindesine, vinorelbine, diaziquone, mechlorethamine, mitomycin C, fludarabine, and cytosine arabinoside, or a combination thereof.

13. The method of claim 1, wherein the chemotherapy is gemcitabine and carboplatin, and the cancer is triple negative breast cancer.

14. A method of treating a patient having triple negative breast cancer (TNBC) therapy comprising:
(i) determining whether the patient's TNBC is immunogenic;
(ii) determining whether the patient can be administered an immunogenic cell death (ICD)-inducing chemotherapy; and,
(iii) if it is determined that the TNBC is immunogenic and an ICD-inducing chemotherapy can be administered to the patient,
a. administering to the patient an effective amount of a CDK4/6 inhibitor having the structure: 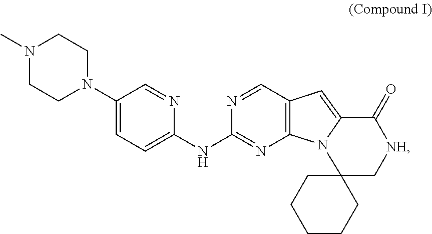 [SMILES: CN1CCN(c2ccc(Nc3ncc4cc5n(c4n3)C3(CCCCC3)CNC5=O)nc2)CC1]
or a pharmaceutically acceptable salt thereof, and,
b. administering to the patient an ICD-inducing chemotherapy regimen comprising gemcitabine and carboplatin;
wherein the CDK4/6 inhibitor is administered prior to administration of the ICD-inducing chemotherapy, and wherein the administration of the CDK4/6 inhibitor and ICD-inducing chemotherapy regimen provides an increase in the progression free survival or overall survival of the patient compared to a patient having an immunogenic TNBC receiving gemcitabine and carboplatin without administration of a CDK4/6 inhibitor.

15. The method of claim 14, wherein the determination of whether the TNBC has a surrounding microenvironment that is favorable to immune modulation comprises comparing the TNBC to those characterized in FIG. 7.

16. The method of claim 14, wherein the determination of whether the cancer has a surrounding microenvironment that is favorable to immune modulation comprises assessing the TNBC according to FIG. 6.

17. The method of claim 14, wherein the determination of whether the TNBC has a surrounding microenvironment that is favorable to immune modulation is based on the Galon immunoscore system.

18. The method of claim 14, wherein the determination of whether the TNBC has a surrounding microenvironment that is favorable to immune modulation comprises assessing whether the TNBC has a sufficiently high level of major histocompatibility complex class I antigens available to initiate an effective immune response.

19. The method of claim 14, wherein the determination of whether the TNBC has a surrounding microenvironment that is favorable to immune modulation comprises assessing whether the TNBC has a sufficiently high level of major histocompatibility complex class II antigens available to initiate an effective immune response.

20. The method of claim 14, wherein the determination of whether the TNBC has a surrounding microenvironment that is favorable to immune modulation comprises assessing whether the TNBC has a sufficiently high level of major histocompatibility complex class I and class II antigens available to initiate an effective immune response.

21. The method of claim 14, wherein the patient has a TNBC that is immunogenically classified as a hot immune tumor.

22. The method of claim 14, wherein the patient has a TNBC that is immunogenically classified as an altered-immunosuppressed tumor.

23. The method of claim 14, wherein the determination of whether the TNBC has a surrounding microenvironment that is favorable to immune modulation comprises assessing whether the TNBC (i) is an IFN-γ Dominant class cancer, (ii) has a microenvironment with a high IFN-γ Signature, (iii) has a high Expanded Immune Signature, (iv) is PD-L1 positive, or a combination thereof.

24. The method of claim 14, wherein the CDK4/6 inhibitor is administered about 4 hours or less prior to the administration of the chemotherapy.

25. A method of treating a patient having triple negative breast cancer (TNBC) therapy comprising:
(i) determining whether the patient's TNBC has a high IFN-γ Signature;
(ii) determining whether the patient can be administered an immunogenic cell death (ICD)-inducing chemotherapy; and,
(iii) if it is determined that the TNBC has a high IFN-γ Signature and an ICD-inducing chemotherapy can be administered to the patient,
a. administering to the patient an effective amount of a CDK4/6 inhibitor having the structure: 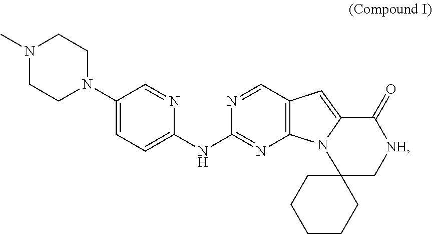 [SMILES: CN1CCN(c2ccc(Nc3ncc4cc5n(c4n3)C3(CCCCC3)CNC5=O)nc2)CC1]
or a pharmaceutically acceptable salt thereof, and,
b. administering to the patient an ICD-inducing chemotherapy regimen comprising gemcitabine and carboplatin;
wherein the CDK4/6 inhibitor is administered prior to administration of the ICD-inducing chemotherapy, and wherein the administration of the CDK4/6 inhibitor and ICD-inducing chemotherapy regimen provides an increase in the progression free survival or overall survival of the patient compared to a patient having an immunogenic TNBC receiving gemcitabine and carboplatin without administration of a CDK4/6 inhibitor.

26. The method of claim 25, wherein the TNBC is an IFN-γ Dominant class TNBC.

27. The method of claim 25, wherein the TNBC has a high Expanded Immune Signature.

28. The method of claim 25, wherein the TNBC is PD-L1 positive.

29. The method of claim 25, wherein the CDK4/6 inhibitor is administered about 4 hours or less prior to the administration of the chemotherapy.
